(19) United States
(12) Patent Application Publication
(10) Pub. No.: US 2003/0044791 A1
(43) Pub. Date: Mar. 6, 2003

(54) Adaptor kits and methods of use

(76) Inventor: Erik Kolstad Flemington, New Orleans, LA (US)

Correspondence Address: Elizabeth Spar, Palmer & Dodge LLP, One Beacon Street, Boston, MA, 02108, US

(21) Appl. No.: 09/880,313

(22) Filed: Jun. 13, 2001

(51) Int. Cl. C12Q 1/68; C07H 21/04; C12P 19/34

(52) U.S. Cl. 435/6; 536/24.3; 435/91.2

(57) ABSTRACT

The invention relates to adaptor molecules and kits and arrays comprising adaptor molecules. The invention also relates to methods of making and using adaptor molecules.

### FIELD OF THE INVENTION

The invention relates in general to adaptor molecules wherein each end of the adaptor is compatible with a nucleic acid digested with a restriction enzyme or a nucleic acid comprising an end that is compatible with a nucleic acid digested with a restriction enzyme. The invention also relates to kits comprising adaptor molecules and their methods of use.

### BACKGROUND OF THE INVENTION

Specific DNA (deoxyribonucleic acid) fragments can be amplified and isolated in high yields by molecular cloning methods well known in the art. Molecular cloning involves the steps of joining the DNA fragments to be cloned in vitro to an autonomously replicating cloning vehicle molecule, e.g., plasmid DNA (Cohen, S. N. et al., PNAS 70, 3240 (1973); Tanaka, T. and Weisblum, B., J. Bacteriology 121, 354 (1975)) or lambda phage DNA (Thomas, M. et al., PNAS 71, 4579 (1974); Murray, N. E. and Murray, K., Nature 251, 476 (1974)) and introducing the hybrid recombinant DNA-cloning vehicles into host cells, e.g., E. coli cells, by transformation. The recombinant DNA-cloning vehicle is then cloned by a suitable technique such as single colony isolation or plaque formation.

In one embodiment, the same restriction endonuclease is used to digest two different DNA molecules to produce identical cohesive ends. The DNA molecules are annealed to one another and then covalently joined by DNA ligase. According to this method, there are limitations on the size and kind of DNA fragments that can be cloned since this method often requires cloning of a much larger DNA fragment that contains the DNA of interest. For example, when cloning a small DNA fragment (e.g., a promoter), the nearest restriction endonuclease sites may be relatively distant, and thus extraneous DNA sequences must be included in the cloned DNA. According to this method, undesirable or even hazardous sequences may be transferred along with the sequence of interest. Furthermore, because of the lack of a suitable restriction enzyme for producing molecules with appropriate cohesive ends, many DNA fragments cannot be cloned by this method.

U.S. Pat. No. 4,321,365 teaches a single pair of chemically synthesized oligonucleotides that can anneal to form an adaptor with two protruding nucleotide sequences which are recognition sites for the same or different restriction endonucleases at opposite ends of the duplex. U.S. Pat. No. 4,321,365 also teaches adaptor molecules comprising annealed oligonucleotides with two protruding nucleotide sequences which are recognition sites for the same or different restriction endonucleases at opposite ends of the duplex.

A single adaptor comprising a first end that is a restriction enzyme recognition site for a blunt cutting restriction enzyme and a second end comprising a protruding nucleotide sequence that is a restriction enzyme recognition site are also known in the art (Bahl et al., 1978, Biochem & Biophys. Res. Comm., 81:695). Phosphorylated forms of the adaptors of U.S. Pat. No. 4,321,365 and Bahl et al., are also known in the art.

There is also a need in the art for a kit comprising two or more adaptor molecules for cloning nucleic acid molecules that does not require the design and synthesis of PCR primers.

There is also a need in the art for a kit comprising two or more adaptor molecules for cloning nucleic acid molecules that does not require the design and synthesis of oligonucleotides. The invention provides for a kit that increases the speed of performing cloning reactions.

### SUMMARY OF THE INVENTION

The present invention provides a kit comprising two or more adaptors in a ready-to use format, wherein each adaptor comprises annealed, chemically synthesized oligonucleotides, and further comprises a nucleotide sequence at each end which is compatible with a sample nucleic acid sequence digested with a restriction endonuclease. The adaptor molecules of the kits of the invention are joined at the ends of natural or synthetic DNA molecules to form adapted DNA molecules. The ends of such natural or synthetic DNA molecules can be blunt-ended or can comprise a protruding nucleotide sequence. The adapted DNA molecules are ligated to a cloning vehicle, thereby making the cloning procedure much more rapid, and efficient and much less error-prone. The adaptors of the invention allow for cloning a first nucleic acid molecule to a second nucleic acid molecule wherein the first and second nucleic acid molecules share only a single set of compatible ends. The other end of each nucleic acid molecule is made compatible by the use of the adaptor of the kit of the invention.

There is a need in the art for a kit comprising two or more adaptor molecules that are in a ready-to-use format. As used herein, “ready-to-use format” refers to a kit wherein each adaptor comprising the kit is provided at a concentration and in a solution such that the adaptor can be added directly to a reaction (for example a ligation reaction) without being subjected to additional manipulations.

The invention provides for a composition comprising a pair of oligonucleotides wherein the first oligonucleotide of the pair comprises in 5′ to 3′ order a first end, a central region, and a second end, and the second oligonucleotide of the pair comprises in 5′ to 3′ order a first end, a central region, and a second end. The central region of the first oligonucleotide of the pair is complementary to at least a portion of the central region of the second oligonucleotide of the pair. The first end of the first oligonucleotide and the second end of the second oligonucleotide of the pair individually comprise a nucleotide sequence that is either the sense or antisense strand of at least a portion of a double stranded recognition sequence that is compatible with a sample nucleic acid digested with a first restriction enzyme. The second end of the first oligonucleotide of the pair and the first end of the second oligonucleotide of the pair individually comprise a nucleotide sequence that is either the sense or antisense strand of at least a portion of a double stranded recognition sequence that is compatible with a sample nucleic acid digested with a second restriction enzyme. According to this embodiment, each of the pair of oligonucleotides is selected from the pairs of oligonucleotides presented in FIGS. 1-5.

As used herein, an “oligonucleotide” refers to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including a basic sites). Preferably, an oligonucleotide according to the invention is single-stranded DNA or RNA. An oligonucleotide of the invention can also be modified. The terms “oligonucleotide” or “nucleic acid” intend a polynucleotide of genomic DNA or RNA, cDNA, semisynthetic, or synthetic origin which, by virtue of its synthetic origin or manipulation: (1) is not associated with all or a portion of the polynucleotide with which it is associated in nature; and/or (2) is linked to a polynucleotide other than that to which it is linked in nature. Oligonucleotides useful according to the invention are between about 6 to 100 nucleotides in length, preferably about 8-50 nucleotides in length and more preferably about 11-25 nucleotides in length. An oligonucleotide according to the invention can be unphosphorylated or phosphorylated at one or both ends.

The invention provides for an oligonucleotide comprising a first end and a second end, and a central region. Preferably, each of the first and second ends of the oligonucleotide individually comprises a nucleotide sequence corresponding to either the sense or antisense strand of a sequence that is compatible with a nucleic acid digested with a restriction enzyme. A first and a second end of an oligonucleotide of the invention are at least one nucleotide in length and preferably less than 99 nucleotides in length. A first end second end of an oligonucleotide are preferably between 1 and 20 nucleotides, more preferably between 1 and 10 nucleotides and most preferably between 1 and 6 nucleotides in length. As used herein, “central region” refers to a portion of the oligonucleotide that does not overlap with the “first end” or “second end” but is located between the first and second ends of the oligonucleotide. At least a portion of the “central region” as it refers to a first oligonucleotide of an oligonucleotide pair, is complementary to at least a portion of the “central region” of a second oligonucleotide of the pair. (see FIG. 6). At least a portion of the “central region” as it refers to a second oligonucleotide of an oligonucleotide pair, is complementary to at least a portion of the “central region” of the first oligonucleotide of the pair. In one embodiment, the “central region” further comprises either the sense or antisense strand of a double stranded start signal or a stop signal, as defined herein. In another embodiment, the “central region” also comprises a subregion encoding from 1 to 25 amino acids, wherein the subregion comprises nucleotides encoding a tag, or encoding a portion of a gene of interest (i.e., a gene from which a nucleic acid molecule to be cloned is derived).

As used herein, a “tag” refers to a sequence of amino acids that encodes a protein or epitope that mediates specific binding to a binding partner (i.e., a receptor, a protein, an antibody or an antigen). A “tag” also includes a fusion of short peptides or protein domains. A “tag” that is useful according to the invention includes but is not limited to histidine, hemagglutinin, glutathione-S-transferase, myc, lacZ, trpE, thioredoxin, FLAG tag and calmodulin-binding tag.

In another embodiment, the “central region” further comprises a subregion encoding any amino acid sequence of interest that maintains an appropriate reading frame.

The invention also provides for a composition comprising at least two adaptors, wherein each adaptor comprises a first protruding nucleotide sequence at one end and a second protruding nucleotide sequence at the opposite end, and each of the first and second protruding nucleotide sequence are individually compatible with a sample nucleic acid digested with a restriction enzyme, and each adaptor is individually compartmentalized.

The invention also provides for a composition comprising at least two adaptors, wherein each adaptor comprises a protruding nucleotide sequence that is compatible with a first sample nucleic acid digested with a restriction enzyme at one end and a second end comprising a sequence that is compatible with a sample nucleic acid digested with a blunt-cutting restriction enzyme at the opposite end.

As used herein, an “adaptor” refers to a double-stranded nucleic acid molecule comprising a pair of annealed oligonucleotides and having a first and a second end. In one embodiment, each end of an adaptor comprises a nucleic acid sequence that is compatible with a nucleic acid digested with a restriction enzyme or a nucleic acid sequence comprising an end that is compatible with a nucleic acid digested with a restriction enzyme. The invention provides for an adaptor wherein either or both ends of the adaptor comprise a protruding nucleotide sequence that is compatible with a nucleic acid digested with a restriction enzyme that generates a sticky end. The invention also provides for an adaptor wherein either or both ends of the adaptor comprise a blunt-ended nucleic acid sequence that is compatible with a nucleic acid digested with a blunt-cutting restriction enzyme. The invention also provides for an adaptor wherein one end of the adaptor comprises a protruding nucleotide sequence that is compatible with a nucleic acid digested with a restriction enzyme that generates a sticky end and the opposite end of the adaptor comprises a blunt-ended nucleic acid sequence that is compatible with a nucleic acid digested with a blunt-cutting restriction enzyme. The invention also provides for adaptors wherein one or both ends comprise a nucleotide sequence that is compatible with a nucleic acid digested with a restriction enzyme that is a “frequent” cutter, an “infrequent” cutter, a “standard” cutter, an enzyme that generates a blunt end, an enzyme that generates a 3′ overhang, an enzyme that generates a 5′ overhang, or a restriction enzyme that digests the polylinker of many commonly used cloning vectors (presented herein) all as defined herein.

The two annealed oligonucleotides comprising an adaptor according to the invention have at least 2 and less than 90 (i.e., 2, 4, 5, 6, 10, 20, 50 etc . . . up to 89) contiguous hybridizing base pairs. Preferably, the two annealed oligonucleotides comprising an adaptor according to the invention have 2-89, preferably 4-50, more preferably 5-30 and most preferably 6-25 contiguous hybridizing base pairs. The invention provides for an adaptor wherein the double stranded nucleotide sequence located between the terminal nucleic acid sequences that are compatible with a nucleic acid digested with a restriction enzyme can be any sequence that includes 2-89, preferably 4-50, more preferably 5-30 and most preferably 6-25 contiguous hybridizing base pairs.

In certain embodiments, one or both ends of an adaptor according to the invention is/are phosphorylated.

As used herein, “anneal” refers to hydrogen bonding with a complementary oligonucleotide or nucleic acid, via an interaction between for example, two oligonucleotides. A pair of oligonucleotides are annealed if they are stably associated to form an adaptor that can be ligated to at least one nucleic acid molecule, preferably via an end that is compatible with an end of the adaptor. Methods of annealing complementary oligonucleotides are described herein in the section entitled, “Adaptors”. The invention provides for annealed oligonucleotides that are in solution or are lyophilized. Methods of using an adaptor according to the invention in a ligation reaction are described herein in the section entitled, “Adaptors”. As used herein, “stably associated” refers to a pair of oligonucleotides that associate with each other with a dissociation constant (K_{D}) of at least about 1×10³ M⁻¹, usually at least 1×10⁴ M⁻¹, typically at least 1×10⁵ M⁻¹, preferably at least 1×10⁶ M⁻¹ to 1×10⁷ M⁻¹ or more.

As used herein, complementary refers to base pairs that bind to each other by hydrogen bonds. Adenine (A) and thymine (T) are complementary base pairs. Cytosine (C) and guanine (G) are also complementary base pairs. As used herein, “complementary” also refers to nucleic acid sequences that can bind to each other by hydrogen bonds between complementary base pairs. For example, the sequences 5′-TCGCAT-3′ and 3′-AGCGTA-5′ are completely complementary according to the invention. The invention also provides for sequences that are partially complementary.

As used herein, “partially complementary” refers to sequences that are less than 100% (i.e., 99%, 90%, 80%, 70%, 60%, 50% etc . . . ) complementary.

As used herein, “compatible” refers to a nucleic acid sequence comprising at least a portion that is complementary to a second nucleic acid sequence. “Compatible” nucleic acid sequences can bind to each other, via hydrogen bonds between complementary base pairs, and can be attached to each other by the formation of a phosphodiester bond between a juxtaposed 5′ phosphate of one sequence and a 3′ hydroxyl terminus of a second sequence, in a ligation reaction, as defined herein, to form a ligation product. In embodiments wherein an “adaptor” connects two nucleic acid molecules, the ligation product is preferably greater in size than either of the individual nucleic acid sequences. In embodiments wherein an “adaptor” connects two ends of a single nucleic acid molecule, the ligation product will be detected by determining if the ligation product can form a colony in a transformation assay, and by detecting a product that is equivalent in size to the ligated nucleic acid from the nucleic acid isolated from the colony. As used herein, “at least a portion of”, as it refers to a nucleic acid sequence, means less than 100%, (e.g., 99%, 90%, 75%, 50%, 25% etc . . . ) of the nucleotides of the nucleic acid sequence. The invention provides for a pair of compatible nucleic acid molecules wherein only a portion of each member of the pair is complementary to the other member of the pair. In embodiments wherein only a portion of a nucleic acid sequence is compatible with a second nucleic acid sequence, the compatible nucleic acid bases can be contiguous or scattered. Two compatible nucleic acid sequences according to the invention can form at least one base pair, preferably two base pairs and most preferably 4 or more (i.e., 5, 6, 10 etc . . . ) but less than 90 base pairs. Two compatible nucleic acid sequences according to the invention have 2-89, preferably 4-50, more preferably 5-30 and most preferably 6-25 contiguous hybridizing base pairs.

The invention also provides for compatible blunt-ended nucleic acids that do not bind to each other via hydrogen bonds between complementary base pairs but can be attached by the formation of a phosphodiester bond between a juxtaposed 5′ phosphate and a 3′ hydroxyl terminus of one or more nucleic acid molecules in a ligation reaction, as defined herein, to form a product that is equivalent in size to the sum of the individual nucleic acid sequences. In embodiments wherein an “adaptor” connects two nucleic acid molecules, the ligation product is preferably greater in size than either of the individual nucleic acid sequences.

The invention also provides for a nucleic acid sequence wherein the first end of the sequence is compatible with the opposite end of the sequence such that the two ends can be attached to each other by the formation of a phosphodiester bond between a juxtaposed 5′ phosphate of one end and a 3′ hydroxyl terminus of the opposite end, in a ligation reaction, as defined herein. In embodiments wherein an “adaptor” connects two ends of a single nucleic acid molecule, the ligation product will be detected in a transformation assay, as described above.

As used herein, a “protruding nucleotide sequence” refers to a single stranded region of a nucleic acid that extends from a double stranded region. A “protruding nucleotide sequence” is from 1-20 nucleotides, preferably from 1-8 nucleotides and most preferably from 1-4 nucleotides in length. A “protruding nucleotide sequence” according to the invention can ligate to the end of a nucleic acid, including but not limited to a nucleic acid that has been digested with a restriction enzyme that digests a double stranded nucleic acid to generate an end that is compatible with the end of the “protruding nucleotide sequence”. Ligation is performed in a ligation reaction, as defined herein, and is detected by the formation of a product that is, preferably, greater in size, as determined by gel electrophoresis on an agarose gel, than any individual component of the ligation reaction.

As used herein, “ligation” means the formation of a phosphodiester bond between a juxtaposed 5′ phosphate and a 3′ hydroxyl terminus of one or more nucleic acid molecules in a ligation reaction.

As used herein, a “nucleic acid ligation activity” refers to an enzyme that catalyzes the formation of a phosphodiester bond between a juxtaposed 5′ phosphate and a 3′ hydroxyl terminus of one or more nucleic acid molecules in a ligation reaction. A nucleic acid ligation activity useful according to the invention includes but is not limited to T4 DNA ligase, E. coli DNA ligase, Taq DNA ligase, Pfu DNA ligase and RNA ligase.

As used herein, a “recognition site for a restriction enzyme” refers to a specific sequence of DNA that is recognized and cleaved by a restriction enzyme either at the recognition site or at a site located upstream or downstream from the recognition site. For example, the recognition site for the restriction enzyme EcoR1 is the double stranded sequence 5′-GAATTC-3′/3′-CTTAAG-5′. The invention also provides for a “portion of a recognition site for a restriction enzyme”. As used herein, a “portion of a recognition site for a restriction enzyme” refers to less than 100% of the recognition site“. For example, AATTC is a portion of the recognition site for EcoR1.

As used herein, a “restriction enzyme” refers to an enzyme that recognizes a specific recognition site or target sequence of DNA and cleaves the DNA, either at the target sequence or at a site located upstream or downstream of the target sequence. The invention provides for restriction enzymes that produce cohesive or sticky ends, or blunt ends. The invention also provides for restriction enzymes that are standard, frequent or infrequent cutters, a restriction enzyme that generates a 5′ overhang, a restriction enzyme that generates a 3′ overhang, and restriction enzymes that digest the polylinkers of many commonly used cloning vectors. A restriction enzyme useful according to the invention includes any restriction enzyme that has been identified (i.e., any restriction enzyme known in the art including but not limited to the restriction enzymes presented in the section entitled “Adaptors”).

A restriction enzyme that produces a cohesive or sticky end makes a staggered cut (i.e., the cut is not made at the midpoint of the recognition sequence) and produces a single stranded nucleic acid overhang on each end of the nucleic acid that has been cut. Complementary single-stranded nucleic acid overhangs can associate by base-pairing and are referred to herein as “cohesive ends” or “sticky ends”. A restriction enzyme that produces a sticky end and is useful according to the invention includes but is not limited to any of the enzymes presented in Table 8.

A “blunt cutting” restriction enzyme cuts a nucleic acid at the midpoint of the recognition sequence to produce blunt-ended fragments that are base paired out to their ends and do not associate with each other. A restriction enzyme that produces a blunt end and is useful according to the invention includes but is not limited to any of the enzymes presented in Table 2.

As used herein, a standard restriction enyzme refers to any enzyme that digests a 6 bp restriction enzyme recognition sequence, wherein there is no nucleotide wobble at the recognition sequence. A “standard restriction enzyme” that is useful according to the invention includes any of the enzymes presented in Table 1.

As used herein, “nucleotide wobble” refers to a position of a defined nucleotide sequence that is not restricted to one specific nucleotide. For example, the sequence 5′-AGGNCCT-3′ contains a “nucleotide wobble” in the fourth position (i.e., the N position) which can be any nucleotide (i.e., A, G, C, or T).

As used herein, a frequent cutting restriction enzyme refers to an enzyme that digests a restriction enzyme recognition sequence comprising less than 6 non-redundant base positions. A frequent-cutting restriction enzyme that is useful according to the invention includes but is not limited to any of the enzymes presented in Table 3.

As used herein, “non-redundant base positions” refers to the sense or antisense nucleotide located at a position within a defined nucleotide sequence that is restricted to a single, specific nucleotide. For example, the sequence 5′-AGGNCCT-3′ contains six “non-redundant” base positions” (i.e., A, G, G (positions 1, 2, 3) and C, C, T (positions 5, 6, 7)) and one “wobble” position (i.e., N).

As used herein, an infrequent cutting restriction enzyme refers to an enzyme that digests a restriction enzyme recognition sequence comprising more than six non-redundant base pairs, wherein the recognition sequence can further include one or more wobble base pairs. An infrequent-cutting restriction enzyme that is useful according to the invention includes but is not limited to any of the enzymes presented in Table 4.

The invention also includes restriction enzymes that generate a 5′ overhang, including but not limited to the enzymes listed in Table 6.

The invention also includes restriction enzymes that generate a 3′ overhang, including but not limited to the enzymes listed in Table 5.

The invention also includes restriction enzymes that digest the polylinker of many commonly used cloning vectors, including but not limited to the restriction enzymes presented in Table 7.

As used herein, “commonly used cloning vectors” include but are not limited to pSP72, pSP73, pSP64, pGEM (Promega), pBluescript II Phagemid Vector, pBT Bait Plasmid (Stratagene), pcDNA3 and pcDNA4 (InVitroGen). Additional “commonly used cloning vectors” useful according to the invention are presented in Table 10.

As used herein, “individually compartmentalized” as it refers to oligonucleotide pairs or adaptors of the invention, means physically separated from every other oligonucleotide pair or adaptor, in a kit, due to containment (i.e., in an eppendorf tube, a well of a plate, such as a 96 well plate, a glass jar, or a capillary tube or on a piece of filter paper).

As used herein, “other” refers to oligonucleotides comprising a oligonucleotide pair or adaptor of a different sequence.

As used herein, a “different sequence” refers to a sequence that is different from another sequence at one or more nucleotides.

In one embodiment, each adaptor comprises an identical first protruding nucleotide sequence that is compatible with a sample nucleic acid digested with a restriction enzyme selected from the group consisting of a standard restriction enzyme, an infrequent-cutting enzyme, a frequent-cutting enzyme, an enzyme that generates a 5′ overhang or an enzyme that generates a 3′ overhang.

In another embodiment, each adaptor comprises an identical nucleotide sequence that is compatible with a sample nucleic acid digested with a blunt-cutting restriction enzyme.

In another embodiment, the first and second protruding nucleotide sequence of each adaptor are individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

In another embodiment, the protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

The invention also provides for a composition comprising at least two adaptors wherein each adaptor comprises a first end and a second end, wherein each of the first and second ends is individually compatible with a sample nucleic acid digested with a restriction enzyme, and each of the at least two adaptors is selected from the adaptors presented in FIGS. 1-5.

In one embodiment, each of the at least two adaptors is phosphorylated.

The invention also provides for an adaptor comprising a pair of annealed oligonucleotides having at one end a first protruding nucleotide sequence and at the opposite end a second protruding nucleotide sequence, wherein the first and second protruding nucleotide sequences are individually compatible with a sample nucleic acid digested with a recognition site for a restriction enzyme. According to this embodiment, the adaptors further comprise at least one stop signal or at least one start signal located between the first and second protruding nucleotide sequence.

The invention also provides for an adaptor comprising a pair of annealed oligonucleotides having at one end a protruding nucleotide sequence that is compatible with a sample nucleic acid digested with a restriction enzyme and at the opposite end a nucleotide sequence that is compatible with a nucleic acid sequence digested with a blunt-cutting restriction enzyme, and further comprising at least one stop signal or at least one start signal located between the first and second protruding nucleotide sequences.

The invention also provides for an adaptor comprising a pair of annealed oligonucleotides wherein each oligonucleotide of the pair has a first and a second end, wherein each of the first and second ends is individually compatible with a sample nucleic acid digested with a restriction enzyme. According to this embodiment, the adaptor is selected from the adaptors presented in FIGS. 1-5.

In one embodiment, the adaptor is phosphorylated.

An adaptor according to the invention can comprise a stop signal or a start signal.

As used herein, a “start signal” refers to a codon that is recognized as the starting point for protein synthesis or translation (i.e., ATG).

As used herein, a “stop signal” refers to a codon that terminates protein synthesis, (i.e., TGA, TAG and TAA).

The invention also provides for an adaptor wherein a start or stop signal is present in each of the three reading frames with respect to the terminal nucleotide sequences that are compatible with a nucleic acid digested with a restriction enzyme.

The invention also provides for a set of three adaptors wherein each adaptor of the set is provided in a different reading frame to maintain the appropriate reading frame after performing a ligation reaction (see for example, FIG. 5).

The invention also provides for an adaptor comprising a sequence that encodes an amino acid sequence. An adaptor that comprises a sequence that encodes any amino acid sequence that maintains the correct reading frame, and is within the size limitations of the adaptor sequence presented herein, is useful according to the invention.

The invention provides for kits and arrays comprising a group of individually compartmentalized oligonucleotide pairs or adaptors wherein the members of a particular group of a kit are selected to facilitate the joining of two or more nucleic acid molecules.

An “array” according to the invention also refers to a group of individually compartmentalized adaptors wherein the members of a particular group of an array are selected to facilitate the joining of two nucleic acid molecules.

The invention provides for a kit comprising two or more adaptor molecules for cloning nucleic acid molecules by a method that is not limited by the size of the nucleic acid fragment being cloned.

The invention also provides for a kit comprising two or more adaptor molecules for cloning nucleic acid molecules by a method that is error free and therefore does not require verification of the cloning by a sequencing step.

The invention also provides for a kit comprising two or more adaptor molecules wherein each adaptor comprises a sequence that is compatible with a nucleic acid sequence digested with a restriction enzyme, located at each end of the adaptor and flanking either a stop or a start signal.

The invention also provides for a kit comprising two or more adaptor molecules wherein each adaptor comprises a restriction enzyme recognition sequence located at each end of the adaptor and flanking a nucleotide sequence encoding either a tag or a portion of a gene of interest, or any amino acid sequence that maintains the appropriate reading frame.

The invention also provides for a kit comprising at least one set of three adaptors, wherein each adaptor molecule of the set has a first end and a second end, and each end is compatible with a nucleic acid sequence digested with a restriction enzyme. Each adaptor of the set comprises one of three possible reading frames (see FIG. 5). These kits are useful for preparing in frame fusion proteins or deletion mutants.

The invention provides for a kit comprising at least two pairs of oligonucleotides wherein each pair of the two pairs is individually compartmentalized. The first oligonucleotide of the pair comprises in 5′ to 3′ order a first end, a central region, and a second end, and the second oligonucleotide of the pair comprises in 5′ to 3′ order a first end, a central region, and a second end, wherein the central region of the first oligonucleotide of the pair is complementary to at least a portion of the central region of the second oligonucleotide of the pair. The first end of the first oligonucleotide and the second end of the second oligonucleotide of the pair individually comprise a nucleotide sequence that is either the sense or antisense strand of at least a portion of a double stranded recognition sequence that is compatible with a sample nucleic acid digested with a first restriction enzyme. The second end of the first oligonucleotide of the pair and the first end of the second oligonucleotide of the pair individually comprise a nucleotide sequence that is either the sense or antisense strand of at least a portion of a double stranded recognition sequence that is compatible with a sample nucleic acid digested with a second restriction enzyme. The kit further comprises packaging means thereof.

In one embodiment, each pair of oligonucleotides is annealed to form an adaptor wherein the adaptor comprises a first protruding nucleotide sequence at one end and a second protruding nucleotide sequence at the opposite end, and each of the first and second protruding nucleotide sequences are compatible with a sample nucleic acid digested with a restriction enzyme.

In another embodiment, each adaptor comprises an identical first protruding nucleotide sequence that is compatible with a sample nucleic acid digested with a restriction enzyme selected from the group consisting of a standard restriction enzyme, a frequent-cutting restriction enzyme or an infrequent-cutting restriction enzyme.

In another embodiment, the first and second protruding nucleotide sequence of each adaptor are individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

In another embodiment, one of the restriction enzymes is a blunt-cutting restriction enzyme.

In another embodiment, each pair of oligonucleotides is annealed to form an adaptor, wherein the adaptor comprises a protruding nucleotide sequence compatible with a sample nucleic acid digested with a restriction enzyme at one end and a nucleotide sequence compatible with a sample nucleic acid digested with a blunt-cutting restriction enzyme at the opposite end.

In another embodiment, each adaptor comprises an identical nucleotide sequence that is compatible with a sample nucleic acid digested with a blunt-cutting restriction enzyme.

In another embodiment, the protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

In another embodiment, each of the at least two pairs of oligonucleotides is selected from the pairs of oligonucleotides presented in FIGS. 1-5.

The invention also provides for a kit comprising at least two adaptors, wherein each of the two adaptors comprises a pair of annealed oligonucleotides and further comprises a first protruding nucleotide sequence at one end and a second protruding nucleotide sequence at the opposite end. Each of the first and second protruding nucleotide sequences are individually compatible with a sample nucleic acid digested with a restriction enzyme. The kit also includes packaging means.

The invention also provides for a kit comprising at least two adaptors, wherein each of the two adaptors comprises a pair of annealed oligonucleotides and further comprises a protruding nucleotide sequence that is compatible with a sample nucleic acid digested with a restriction enzyme at one end and a sample nucleic acid digested with a blunt-cutting restriction enzyme at the opposite end, and packaging means.

In one embodiment, each adaptor comprises an identical first protruding nucleotide sequence that is compatible with a sample nucleic acid digested with a restriction enzyme, selected from the group consisting of a standard restriction enzyme, an infrequent-cutting enzyme, a frequent-cutting enzyme, an enzyme that generates a 5′ overhang or an enzyme that generates a 3′ overhang.

In another embodiment, each adaptor comprises an identical nucleotide sequence that is compatible with a sample nucleic acid digested with a blunt-cutting restriction enzyme.

In another embodiment, the first and second protruding nucleotide sequence of each adaptor are individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

In another embodiment, the protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

The invention also provides for a kit comprising at least two adaptors, wherein each of the at least two adaptors comprises a first end and a second end, and each of the first and second ends are individually compatible with a sample nucleic acid digested with a restriction enzyme. According to this embodiment, each of the at least two adaptors is selected from the adaptors presented in FIGS. 1-5.

In one embodiment, each of the at least two adaptors is phosphorylated.

The invention also provides for an array comprising at least two pairs of oligonucleotides wherein each pair of the two pairs is individually compartmentalized, wherein the first oligonucleotide of the pair comprises in 5′ to 3′ order a first end, a central region, and a second end, and the second oligonucleotide of the pair comprises in 5′ to 3′ order a first end, a central region, and a second end. The central region of the first oligonucleotide of the pair is complementary to at least a portion of the central region of the second oligonucleotide of the pair. The first end of the first oligonucleotide and the second end of the second oligonucleotide of the pair individually comprises a nucleotide sequence that is either the sense or antisense strand of at least a portion of a double stranded recognition sequence that is compatible with a sample nucleic acid digested with a first restriction enzyme. The second end of the first oligonucleotide of the pair and the first end of the second oligonucleotide of the pair individually comprises a nucleotide sequence that is either the sense or antisense strand of at least a portion of a double stranded recognition sequence that is compatible with a sample nucleic acid digested with a second restriction enzyme.

In one embodiment, each pair of oligonucleotides is annealed to form an adaptor wherein the adaptor comprises a first protruding nucleotide sequence at one end and a second protruding nucleotide sequence at the opposite end, and each of the first and second protruding nucleotide sequences is compatible with a sample nucleic acid digested with a restriction enzyme.

In another embodiment, each adaptor comprises an identical first protruding nucleotide sequence that is compatible with a sample nucleic acid digested with a restriction enzyme wherein said restriction enzyme is selected from the group consisting of a standard restriction enzyme, a frequent-cutting restriction enzyme, an infrequent-cutting restriction enzyme, an enzyme that generates a 5′ overhang or an enzyme that generates a 3′ overhang.

In another embodiment, the first and second protruding nucleotide sequence of each adaptor are individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

In another embodiment, one of the restriction enzymes is a blunt-cutting restriction enzyme.

In another embodiment, each pair of oligonucleotides is annealed to form an adaptor, wherein the adaptor comprises a protruding nucleotide sequence compatible with a sample nucleic acid digested with a restriction enzyme at one end and a nucleotide sequence compatible with a sample nucleic acid digested with a blunt-cutting restriction enzyme at the opposite end.

In another embodiment, each adaptor comprises an identical nucleotide sequence that is compatible with a sample nucleic acid digested with a blunt-cutting restriction enzyme.

In another embodiment, the protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

In another embodiment, each of the at least two pairs of oligonucleotides is selected from the pairs of oligonucleotides presented in FIGS. 1-5.

The invention also provides for a kit comprising at least two adaptors, wherein each of the two adaptors comprises a pair of annealed oligonucleotides and each of the two adaptors further comprises a first protruding nucleotide sequence at one end and a second protruding nucleotide sequence at the opposite end. Each of the first and second protruding nucleotide sequence is individually compatible with a sample nucleic acid digested with a restriction enzyme. The kit also includes packaging means.

The invention also provides for a kit comprising at least two adaptors, wherein each of the two adaptors comprises a pair of annealed oligonucleotides and further comprises a protruding nucleotide sequence that is compatible with a sample nucleic acid digested with a restriction enzyme at one end and a sample nucleic acid digested with a blunt-cutting restriction enzyme at the opposite end. The kit also includes packaging means.

In one embodiment, each adaptor comprises an identical first protruding nucleotide sequence that is compatible with a sample nucleic acid digested with a restriction enzyme, wherein the restriction enzyme is selected from the group consisting of a standard restriction enzyme, an infrequent-cutting enzyme, a frequent-cutting enzyme, an enzyme that generates a 5′ overhang or an enzyme that generates a 3′ overhang.

In another embodiment, each adaptor comprises an identical nucleotide sequence that is compatible with a sample nucleic acid digested with a blunt-cutting restriction enzyme.

In another embodiment, the first and second protruding nucleotide sequence of each adaptor is individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

In another embodiment, the protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

The invention also provides for a kit comprising at least two adaptors, wherein each of the at least two adaptors comprises a first end and a second end, and each of the first and second ends are individually compatible with a sample nucleic acid digested with a restriction enzyme. According to this embodiment, each of the at least two adaptors is selected from the adaptors presented in FIGS. 1-5.

For example, the invention provides for kits and arrays comprising the following oligonucleotide pairs:

a) the first and second oligonucleotide of the oligonucleotide pair each comprise a first and a second end comprising a nucleotide sequence that is either the sense or antisense strand of a double stranded sequence that is compatible with a nucleic acid sequence digested with a restriction enzyme;

b) the first and second oligonucleotide of the oligonucleotide pair each comprise a first and a second end comprising a nucleotide sequence that is either the sense or antisense strand of a double stranded sequence that is compatible with a nucleic acid sequence digested with a restriction enzyme that is a standard restriction enzyme, an infrequent-cutting restriction enzyme, a frequent-cutting restriction enzyme, a restriction enzyme that generates a blunt end, a restriction enzyme that generates a 3′ overhang, a restriction enzyme that generates a 5′ overhang, or a restriction enzyme that digests the polylinker of many commonly used cloning vectors.

c) the first and second oligonucleotide of the oligonucleotide pair each comprise a first and a second end comprising a nucleotide sequence that is either the sense or antisense strand of a double stranded sequence that is compatible with a nucleic acid sequence digested with a restriction enzyme that is a standard restriction enzyme, an infrequent-cutting restriction enzyme or a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and the first and second oligonucleotide of the pair each comprise a second end that is either the sense or antisense strand of a double stranded sequence that is compatible with a nucleic acid digested with a blunt-cutting restriction enzyme;

d) the first and second oligonucleotide of the oligonucleotide pair each comprise a first and a second end comprising a nucleotide sequence that is either the sense or antisense strand of a double stranded sequence that is compatible with a nucleic acid sequence digested with a restriction enzyme that is a standard restriction enzyme, an infrequent-cutting restriction enzyme or a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and the first and second oligonucleotide of the pair each comprise a second end that is either the sense or antisense strand of a double stranded sequence that is compatible with a nucleic acid digested with a standard restriction enzyme;

e) the first and second oligonucleotide of the oligonucleotide pair each comprise a first and a second end comprising a nucleotide sequence that is either the sense or antisense strand of a double stranded sequence that is compatible with a nucleic acid sequence digested with a restriction enzyme that is a standard restriction enzyme, an infrequent-cutting restriction enzyme or a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and the first and second oligonucleotide of the pair each comprise a second end that is either the sense or antisense strand of a double stranded sequence that is compatible with a nucleic acid digested with a frequent cutting restriction enzyme;

f) the first and second oligonucleotide of the oligonucleotide pair each comprise a first and a second end comprising a nucleotide sequence that is either the sense or antisense strand of a double stranded sequence that is compatible with a nucleic acid sequence digested with a restriction enzyme that is a standard restriction enzyme, an infrequent-cutting restriction enzyme or a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and the first and second oligonucleotide of the pair each comprise a second end that is either the sense or antisense strand of a double stranded sequence that is compatible with a nucleic acid digested with an infrequent-cutting restriction enzyme;

g) the first and second oligonucleotide of the oligonucleotide pair each comprise a first and a second end comprising a nucleotide sequence that is either the sense or antisense strand of a double stranded sequence that is compatible with a nucleic acid sequence digested with a restriction enzyme that is a standard restriction enzyme, an infrequent-cutting restriction enzyme or a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and the first and second oligonucleotide of the pair each comprise a second end that is either the sense or antisense strand of a double stranded sequence that is compatible with a nucleic acid digested with a restriction enzyme that generates a 5′ overhang;

h) the first and second oligonucleotide of the oligonucleotide pair each comprise a first and a second end comprising a nucleotide sequence that is either the sense or antisense strand of a double stranded sequence that is compatible with a nucleic acid sequence digested with a restriction enzyme that is a standard restriction enzyme, an infrequent-cutting restriction enzyme or a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and the first and second oligonucleotide of the pair each comprise a second end that is either the sense or antisense strand of a double stranded sequence that is compatible with a nucleic acid digested with a restriction enzyme that generates a 3′ overhang;

i) the first and second oligonucleotide of the oligonucleotide pair each comprise a first and a second end comprising a nucleotide sequence that is either the sense or antisense strand of a double stranded sequence that is compatible with a nucleic acid sequence digested with a restriction enzyme that is a standard restriction enzyme, an infrequent-cutting restriction enzyme or a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and the first and second oligonucleotide of the pair each comprise a second end that is either the sense or antisense strand of a double stranded sequence that is compatible with a nucleic acid digested with a restriction enzyme that digests the polylinker of many commonly used cloning vectors;

j) the first and second oligonucleotide of the oligonucleotide pair each comprise a first and a second end comprising a nucleotide sequence that is either the sense or antisense strand of a double stranded sequence that is compatible with a nucleic acid sequence digested with a blunt-cutting restriction enzyme;

k) the first and second oligonucleotide of the oligonucleotide pair each comprise a first and a second end comprising a nucleotide sequence that is either the sense or antisense strand of a double stranded sequence that is compatible with a nucleic acid sequence digested with a standard restriction enzyme;

l) the first and second oligonucleotide of the oligonucleotide pair each comprise a first and a second end comprising a nucleotide sequence that is either the sense or antisense strand of a double stranded sequence that is compatible with a nucleic acid sequence digested with an infrequent-cutting restriction enzyme;

m) the first and second oligonucleotide of the oligonucleotide pair each comprise a first and a second end comprising a nucleotide sequence that is either the sense or antisense strand of a double stranded sequence that is compatible with a nucleic acid sequence digested with a frequent-cutting restriction enzyme;

n) the first and second oligonucleotide of the oligonucleotide pair each comprises a first and a second end comprising a nucleotide sequence that is either the sense or antisense strand of a double stranded sequence that is compatible with a nucleic acid sequence digested with a restriction enzyme that generates a 5′ overhang;

o) the first and second oligonucleotide of the oligonucleotide pair each comprises a first and a second end comprising a nucleotide sequence that is either the sense or antisense strand of a double stranded sequence that is compatible with a nucleic acid sequence digested with a restriction enzyme that generates a 3′ overhang; and

p) the first and second oligonucleotide of the oligonucleotide pair each comprises a first and a second end comprising a nucleotide sequence that is either the sense or antisense strand of a double stranded sequence that is compatible with a nucleic acid sequence digested with a restriction enzyme that digests the polylinker of many commonly used cloning vectors.

The invention also provides for kits and arrays comprising oligonucleotide pairs wherein the first and second end of the oligonucleotide pair each comprise a first and a second end comprising a nucleotide sequence that is either the sense or antisense strand of a sequence that is compatible with a nucleic acid sequence digested with a restriction enzyme, and further comprising at least one stop signal or at least one start signal.

For example, the invention also provides for kits and arrays comprising the following adaptors:

a) Each adaptor comprises a first and a second end comprising a nucleotide sequence that is compatible with a nucleic acid digested with a restriction enzyme;

b) Each adaptor comprises a first and a second end compatible with a nucleic acid digested with a restriction enzyme that is a standard restriction enzyme, an infrequent-cutting restriction enzyme, a frequent-cutting restriction enzyme, a restriction enzyme that generates a blunt end, a restriction enzyme that generates a 3′ overhang, a restriction enzyme that generates a 5′ overhang, or a restriction enzyme that digests the polylinker of many commonly used cloning vectors.

c) Each adaptor comprises a first end that is compatible with a nucleic acid digested with a restriction enzyme that is a standard restriction enzyme, an infrequent-cutting restriction enzyme, a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and a second end that is compatible with a nucleic acid digested with a blunt-cutting restriction enzyme;

d) Each adaptor comprises a first end that is compatible with a nucleic acid digested with a restriction enzyme that is a standard restriction enzyme, an infrequent-cutting restriction enzyme, a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and a second end that is compatible with a nucleic acid digested with a standard restriction enzyme;

e) Each adaptor comprises a first end that is compatible with a nucleic acid digested with a restriction enzyme that is a standard restriction enzyme, an infrequent-cutting restriction enzyme, a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and a second end that is compatible with a nucleic acid digested with a frequent-cutting restriction enzyme;

f) Each adaptor comprises a first end that is compatible with a nucleic acid digested with a restriction enzyme that is a standard restriction enzyme, an infrequent-cutting restriction enzyme, a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and a second end that is compatible with a nucleic acid digested with an infrequent-cutting restriction enzyme;

g) Each adaptor comprises a first end that is compatible with a nucleic acid digested with a restriction enzyme that is a standard restriction enzyme, an infrequent-cutting restriction enzyme, a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and a second end that is compatible with a nucleic acid digested with a restriction enzyme that generates a 5′ overhang;

h) Each adaptor comprises a first end that is compatible with a nucleic acid digested with a restriction enzyme that is a standard restriction enzyme, an infrequent-cutting restriction enzyme, a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and a second end that is compatible with a nucleic acid digested with a restriction enzyme that generates a 3′ overhang;

i) Each adaptor comprises a first end that is compatible with a nucleic acid digested with a restriction enzyme that is a standard restriction enzyme, an infrequent-cutting restriction enzyme, a frequent-cutting restriction enzyme, a restriction enzyme that generates a 3′ overhang, a restriction enzyme that generates a 5′ overhang, or a restriction enzyme that digests the polylinker of many commonly used cloning vectors, as defined herein, and a second end that is compatible with a nucleic acid digested with a restriction enzyme that digests the polylinker of many commonly used cloning vectors;

j) Each adaptor comprises a first and a second end compatible with a nucleic acid digested with a blunt-cutting restriction enzyme;

k) Each adaptor comprises a first and a second end compatible with a nucleic acid digested with a standard restriction enzyme;

l) Each adaptor comprises a first and a second end compatible with a nucleic acid digested with an infrequent-cutting restriction enzyme;

m) Each adaptor comprises a first and a second end compatible with a nucleic acid digested with a frequent-cutting restriction enzyme;

n) Each adaptor comprises a first and a second end compatible with a nucleic acid digested with a restriction enzyme that generates a 5′ overhang;

o) Each adaptor comprises a first and a second end compatible with a nucleic acid digested with a restriction enzyme that generates a 3′ overhang; and

p) Each adaptor comprises a first and a second end compatible with a nucleic acid digested with a restriction enzyme that digests the polylinker of many commonly used cloning vectors.

The invention also provides for kits and arrays comprising adaptors wherein the first and second end of the adaptor each comprise a first and a second end comprising a nucleotide sequence that is either the sense or antisense strand of a sequence that is compatible with a nucleic acid sequence digested with a restriction enzyme, and further comprising at least one stop signal or at least one start signal.

The invention provides for a kit comprising at least two adaptors, wherein each of the at least two adaptors comprises a first end and a second end, and each of the first and second ends are individually compatible with a sample nucleic acid digested with a restriction enzyme selected from the group consisting of a standard restriction enzyme, an infrequent cutting enzyme, a frequent cutting enzyme, an enzyme that generates a 5′ overhang, an enzyme that generates a 3′ overhang or a blunt-cutting restriction enzyme, and wherein the adaptor further comprises at least one start signal or at least one stop signal.

The invention also provides for a kit comprising at least one set of three adaptors, wherein each adaptor of the set comprises an identical first end and an identical second end, and each of the first and second ends are individually compatible with a sample nucleic acid digested with a restriction enzyme selected from the group consisting of a standard restriction enzyme, an infrequent cutting enzyme, a frequent cutting enzyme, an enzyme that generates a 5′ overhang, an enzyme that generates a 3′ overhang or a blunt-cutting restriction enzyme. Each of the first and second ends of each adaptor of a set are provided in a different reading frame.

In one embodiment, each of the at least two adaptors is phosphorylated.

The invention also provides for a collection of at least two pairs of oligonucleotides wherein each pair of the two pairs is individually compartmentalized. According to this embodiment, the first oligonucleotide of the pair comprises in 5′ to 3′ order a first end, a central region, and a second end, and the second oligonucleotide of the pair comprises in 5′ to 3′ order a first end, a central region, and a second end. According to this embodiment, the central region of the first oligonucleotide of the pair is complementary to at least a portion of the central region of the second oligonucleotide of the pair. The first end of the first oligonucleotide and the second end of the second oligonucleotide of the pair individually comprise a nucleotide sequence that is either the sense or antisense strand of at least a portion of a double stranded recognition sequence that is compatible with a sample nucleic acid digested with a first restriction enzyme. The second end of the first oligonucleotide of the pair and the first end of the second oligonucleotide of the pair individually comprise a nucleotide sequence that is either the sense or antisense strand of at least a portion of a double stranded recognition sequence that is compatible with a sample nucleic acid digested with a second restriction enzyme.

In one embodiment, each pair of oligonucleotides is annealed to form an adaptor wherein the adaptor comprises a first protruding nucleotide sequence at one end and a second protruding nucleotide sequence at the opposite end, and each of the first and second protruding nucleotide sequences is compatible with a sample nucleic acid digested with a restriction enzyme.

In another embodiment, each adaptor comprises an identical first protruding nucleotide sequence that is compatible with a sample nucleic acid digested with a restriction enzyme wherein the restriction enzyme is selected from the group consisting of a standard restriction enzyme, a frequent-cutting restriction enzyme, an infrequent-cutting restriction enzyme, an enzyme that generates a 5′ overhang or an enzyme that generates a 3′ overhang.

In another embodiment, the first and second protruding nucleotide sequence of each adaptor are individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

In another embodiment, one of the restriction enzymes is a blunt-cutting restriction enzyme.

In another embodiment, each pair of oligonucleotides is annealed to form an adaptor, wherein the adaptor comprises a protruding nucleotide sequence compatible with a sample nucleic acid digested with a restriction enzyme at one end and a nucleotide sequence compatible with a sample nucleic acid digested with a blunt-cutting restriction enzyme at the opposite end.

In another embodiment, each adaptor comprises an identical nucleotide sequence that is compatible with a sample nucleic acid digested with a blunt-cutting restriction enzyme.

In another embodiment, the protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

In another embodiment, each of the at least two pairs of oligonucleotides is selected from the pairs of oligonucleotides presented in FIGS. 1-5.

The invention also provides for a method of using an adaptor comprising providing two nucleic acid molecules, each molecule having a 3′ end and a 5′ end wherein at least the 3′ or the 5′ end of the molecule is digested with a restriction enzyme, and an adaptor comprising two annealed oligonucleotides. One end of the adaptor is compatible with one end of the first nucleic acid molecule, and the opposite end of the adaptor is compatible with one end of the second nucleic acid molecule. The nucleic acid molecules are incubated with the adaptor and with a nucleic acid ligating activity under conditions wherein one end of the adaptor ligates to the first nucleic acid molecule and the opposite end of the adaptor ligates to the second nucleic acid molecule. A ligation product is detected.

The invention also provides for a method of using a kit of the invention comprising the following steps. Two nucleic acid molecules, each molecule having a 3′ end and a 5′ end wherein at least the 3′ or the 5′ end of the molecule is digested with a restriction enzyme are provided. A pair of oligonucleotides is selected from the kit and annealed to form an adaptor, wherein one end of the adaptor is compatible with one end of the first nucleic acid molecule, and the opposite end of the adaptor is compatible with one end of the second nucleic acid molecule. The nucleic acid molecules and the adaptor are incubated with a nucleic acid ligating activity under conditions wherein one end of the adaptor ligates to the first nucleic acid molecule and the opposite end of the adaptor ligates to the second nucleic acid molecule. A ligation product is detected.

The kits and arrays of the invention can also be used for ligating two ends of a single nucleic acid molecule.

The invention also provides for a method of using an array of the invention comprising providing two nucleic acid molecules, each molecule having a 3′ end and a 5′ end wherein at least the 3′ or the 5′ end of the molecule is digested with a restriction enzyme, selecting a pair of oligonucleotides from the array and annealing the pair to form an adaptor, wherein one end of the adaptor is compatible with one end of the first nucleic acid molecule, and the opposite end of the adaptor is compatible with one end of the second nucleic acid molecule. The nucleic acid molecules and the adaptor are incubated with a nucleic acid ligating activity under conditions wherein one end of the adaptor ligates to the first nucleic acid molecule and the opposite end of the adaptor ligates to the second nucleic acid molecule. A ligation product is detected.

The invention also provides for a method of using the kits of the invention comprising providing two nucleic acid molecules, each molecule having a 3′ end and a 5′ end wherein at least the 3′ or the 5′ end of the molecule is digested with a restriction enzyme, and selecting an adaptor from the kit. The adaptor comprises two annealed oligonucleotides, wherein one end of the adaptor is compatible with one end of the first nucleic acid molecule, and the opposite end of the adaptor is compatible with one end of the second nucleic acid molecule. The nucleic acid molecules and the adaptor are incubated with a nucleic acid ligating activity under conditions wherein one end of the adaptor ligates to the first nucleic acid molecule and the opposite end of the adaptor ligates to the second nucleic acid molecule. A ligation product is detected.

The invention also provides for a method of preparing an adaptor comprising the steps of mixing a pair of oligonucleotides, wherein the first oligonucleotide of the pair comprises in 5′ to 3′ order a first end, a central region, and a second end, and the second oligonucleotide of the pair comprises in 5′ to 3′ order a first end, a central region, and a second end. The central region of the first oligonucleotide of the pair is complementary to at least a portion of the central region of the second oligonucleotide of the pair. The first end of each of the oligonucleotide of the pair individually comprises a nucleotide sequence that is either the sense or antisense strand of at least a portion of a double stranded recognition sequence that is compatible with a first sample nucleic acid digested with a first restriction enzyme. The second end of each of the oligonucleotides of the pair individually comprises a nucleotide sequence that is either the sense or antisense strand of at least a portion of a double stranded recognition sequence that is compatible with a second sample nucleic acid digested with a second restriction enzyme. According to this embodiment, one of the restriction enzymes is a blunt-cutting restriction enzyme. The oligonucleotides of each pair are annealed to each other to form an adaptor.

In one embodiment, the adaptor comprises a first protruding nucleotide sequence compatible with a sample nucleic acid digested with a restriction enzyme at one end, and a nucleotide sequence compatible with a sample nucleic acid digested with a blunt-cutting restriction enzyme at the opposite end.

In another embodiment, the restriction enzyme that is not a blunt-cutting restriction enzyme is selected from the group consisting of a standard restriction enzyme, a frequent cutting enzyme, an infrequent-cutting enzyme, an enzyme that generates a 5′ overhang or an enzyme that generates a 3′ overhang.

### BRIEF DESCRIPTION OF THE FIGURES

FIG. 1 demonstrates the oligonucleotide pairs and adaptors comprising a standard adaptor kit of the invention.

FIG. 2 demonstrates the oligonucleotide pairs and adaptors comprising a blunt adaptor kit of the invention.

FIG. 3 demonstrates the oligonucleotide pairs and adaptors comprising a NotI kit of the invention.

FIG. 4 demonstrates the oligonucleotide pairs and adaptors comprising a NotI-stop kit of the invention.

FIG. 5 demonstrates the oligonucleotide pairs and adaptors comprising a BamH1 Reading Frame Alignment kit of the invention.

FIG. 6 demonstrates oligonucleotides/adaptor pairs of the invention wherein the first end, the central region and the second end of each oligonucleotide of the oligonucleotide pair/adaptor are indicated with a bracket.

### DETAILED DESCRIPTION OF THE INVENTION

Cloning methods and kits for carrying out such methods are well known in the art. The invention provides for a kit comprising adaptors provided in a ready-to-use format that allows an investigator to design and perform a cloning experiment without having to wait for the synthesis and annealling of oligonucleotides required to generate an adaptor necessary for the cloning procedure. The adaptors of the invention are grouped into kits or arrays to facilitate the joining of two nucleic acid molecules.

I. Ready-to Use Format

The invention provides for a kit comprising adaptor molecules supplied in a ready-to use format. The adaptors of the kits of the invention can be added directly to a reaction (for example a ligation reaction) without being manipulated (for example, diluted, concentrated, or transferred to another buffer) any further.

Each adaptor of a kit of the invention is supplied in an individualized compartment such that a single adaptor of a particular oligonucleotide pair composition can be added to a reaction of interest in the absence of all other adaptors (for example adaptors comprising oligonucleotide pairs of a different sequence from the adaptor of interest).

A critical feature of an adaptor supplied in a ready-to use format is that the adaptor comprises a previously annealed oligonucleotide pair. That is, the adaptors of the kits of the invention are annealed prior to packaging in the kit. Annealing is performed as described below in the section entitled “Adaptors”. The kit of the invention therefore does not require that the oligonucleotide pairs comprising an adaptor of interest be annealed by the researcher prior to using the adaptors.

Each adaptor of a kit of the invention is supplied at a concentration such that the adaptor can be added directly to the reaction of interest without being diluted or concentrated. The concentration of the adaptor in the kit supports, and is preferably optimal, for the reaction of interest to be performed with the adaptor. The concentration of an adaptor in a kit is from 100 ng/μl to 0.001 ng/μl, preferably from 10 ng/μl to 0.01 ng/μl, and most preferably from 5 ng/μl to 0.1 ng/μl.

Each adaptor of a kit of the invention is supplied in an appropriate buffer such that the adaptor can be added directly to the reaction of interest in the adaptor storage buffer. An appropriate buffer includes any buffer that supports and is preferably optimal for the reaction of interest (for example a ligation reaction). Suitable adaptor storage buffers comprise a salt concentration of less than 1M, preferably less than 500 mM and most preferably less than 200 mM. The pH of a suitable adaptor storage buffer of the invention is between 7 and 8, preferably between 7.2 and 7.8 and most preferably between 7.4 and 7.6. A suitable adaptor storage buffer useful according to the invention includes but is not limited to medium restriction buffer (MRB) (defined herein).

In certain embodiments of the invention, an adaptor that is supplied in a ready-to use format comprise an adaptor wherein one or both ends of the adaptor is phosphorylated.

II. Oligonucleotides

The invention provides for oligonucleotides that can be annealed to form an adaptor according to the invention. The oligonucleotides of the invention comprise a first end, a second end and a central region.

A. Design of Oligonucleotides

An oligonucleotide useful according to the invention is a single-stranded DNA or RNA molecule that is hybridizable to another oligonucleotide to form an adaptor according to the invention.

An oligonucleotide according to the invention is designed to include a first end and a second end that each individually comprise a nucleotide sequence corresponding to either the sense or antisense strand of a double stranded recognition sequence that is compatible with a nucleic acid digested with a restriction enzyme. An oligonucleotide according to the invention is also designed to include a central region that is located between the first and second ends. The invention provides for a pair of oligonucleotides that hybridize to each other, wherein at least a portion of the central region of the first oligonucleotide of the pair is complementary to at least a portion of the central region of the second oligonucleotide of the pair and wherein at least a portion of the central region of the second oligonucleotide of the pair is complementary to at least a portion of the central region of the first oligonucleotide of the pair. The central region can be designed to include a nucleic acid sequence comprising either the sense or antisense strand of a double stranded start or stop signal. In one embodiment, the central region comprises a nucleic acid sequence comprising either the sense or antisense strand of a sequence encoding any amino acid sequence of interest that maintains the proper reading frame. In one embodiment, the central region can be designed to include a nucleic acid sequence that is derived from a gene of interest or that encodes 1 to 25 amino acids comprising a tag.

According to the invention, the oligonucleotide sequences may be chemically or biochemically modified or may contain non-natural or derivatized nucleotide bases. Such modifications include, for example, labels, methylation, substitution of one or more of the naturally occurring nucleotides with an analog, internucleotide modifications such as uncharged linkages (e.g. methyl phosphonates, phosphorodithioates, etc.), pendent moieties (e.g., polypeptides), intercalators, (e.g. acridine, psoralen, etc.) chelators, alkylators, and modified linkages (e.g. alpha anomeric nucleic acids, etc.) Also included are synthetic molecules that mimic oligonucleotides in their ability to bind to a designated sequence via hydrogen bonding and other chemical interactions. Such molecules are known in the art and include, for example, those in which peptide linkages substitute for phosphate linkages in the backbone of the molecule.

The oligonucleotide may be a naturally occurring oligonucleotide, or may be a structurally related variant of such a oligonucleotide having modified bases and/or sugars and/or linkages. The term “oligonucleotide” as used herein is intended to cover all such variants.

Modifications, which may be made to the oligonucleotide may include (but are not limited to) the following types:

a) Backbone modifications

i) phosphorothioates (X or Y or W or Z=S or any combination of two or more with the remainder as O).

e.g. Y=S (Stein et al., 1988, Nucleic Acids Res., 15:3209), X=S (Cosstick and Vyle, 1989, Tetrahedron Letters, 30:4693), Y and Z=S (Brill et al., 1989, J. Amer. Chem. Soc., 111:2321)

ii) methylphosphonates (eg Z=methyl (Miller et al., 1980, J. Biol. Chem., 255:9569))

iii) phosphoramidates (Z=N-(alkyl)₂ e.g. alkyl methyl, ethyl, butyl) (Z=morpholine or piperazine) (Agrawal et al., 1988, Proc. Natl. Acad. Sci., USA, 85;7079) (X or W=NH) (Mag and Engels, 1988, Nucleic Acids Res., 16:3525)

iv) phosphotriesters (Z=O-alkyl e.g. methyl, ethyl etc) (Miller et al., Biochemistry, 21:5468)

v) phosphorus-free linkages (e.g. carbamate, acetamidate, acetate) (Gait et al., 1974, J. Chem. Soc. Perkin I, 1684, Gait et al., 1979, J. Chem. Soc. Perkin I, 1389)

b) Sugar modifications

i) 2′-deoxynucleosides (R=H)

ii) 2′-O-methylated nucleosides (R=OMe) (Sproat et al., 1989, Nucleic Acids Res., 17: 3373)

iii) 2′-fluoro-2′-deoxynucleosides (R=F) (Krug et al., 1989, Nucleosides and Nucleotides, 8:1473)

c) Base modifications—(for a review see Jones, 1979, Int. J. Biolog. Macromolecules, 1:194)

i) pyrimidine derivatives substituted in the 5-position (e.g. methyl, bromo, fluoro etc) or replacing a carbonyl group by an amino group (Piccirilli et al., 1990, Nature, 343:33).

ii) purine derivatives lacking specific nitrogen atoms (e.g. 7-deaza adenine, hypoxanthine) or functionalized in the 8-position (e.g. 8-azido adenine, 8-bromo adenine)

d) Oligonucleotides covalently linked to reactive functional groups, e.g.:

i) psoralens (Miller et al., 1988, Nucleic Acids Res. Special Pub. No. 20:113, phenanthrolines (Sun et al., 1988, Biochemistry, 27:6039), mustards (Vlassov et al., 1988, Gene, 72:313) (irreversible cross-linking agents with or without the need for co-reagents)

ii) acridine (intercalating agents) (Helene et al., 1985, Biochimie, 67:777)

iii) thiol derivatives (reversible disulphide formation with proteins) (Connolly and Newman, 1989, Nucleic Acids Res., 17:4957)

iv) aldehydes (Schiff's base formation)

v) azido, bromo groups (UV cross-linking)

vi) ellipticines (photolytic cross-linking) (Perrouault et al., 1990, Nature, 344:358)

e) Oligonucleotides covalently linked to lipophilic groups or other reagents capable of improving uptake by cells, e.g.:

i) cholesterol (Letsinger et al., 1989, Proc. Natl. Acad. Sci. USA, 86:6553), polyamines (Lemaitre et al., 1987, Proc. Natl. Acad. Sci. USA, 84: 648), other soluble polymers (e.g. polyethylene glycol)

f) Oligonucleotides containing alpha-nucleosides (Morvan et al., Nucleic Acids Res., 15: 3421)

g) Combinations of modifications a)-f)

It is contemplated that oligonucleotides according to the invention are prepared by synthetic methods, either chemical or enzymatic. Alternatively, such a molecule or a fragment thereof is naturally-occurring, and is isolated from its natural source or purchased from a commercial supplier.

Oligonucleotides are between about 6 to 100 nucleotides in length, preferably 8 to 50 nucleotides in length, and most preferably about 11-25 nucleotides in length, although oligonucleotides of different length are of use.

Typically, selective hybridization (i.e., of two oligonucleotides) occurs when two nucleic acid sequences are substantially complementary (at least about 65% complementary over a stretch of at least 14 to 25 nucleotides, preferably at least about 75%, more preferably at least about 90% complementary). See Kanehisa, M., 1984, Nucleic Acids Res. 12: 203, incorporated herein by reference. Preferably, an oligonucleotide pair that anneals to form an adaptor according to the invention comprises at least 2 and less than 90, contiguous hybridizing base pairs.

Numerous factors influence the efficiency and selectivity of hybridization of a first oligonucleotide to a second oligonucleotide. These factors, which include oligonucleotide length, nucleotide sequence and/or composition, hybridization temperature, buffer composition and potential for steric hindrance in the region to which the primer is required to hybridize, will be considered when designing oligonucleotides according to the invention.

A positive correlation exists between oligonucleotide length and both the efficiency and accuracy with which an oligonucleotide will anneal to a complementary oligonucleotide. In particular, longer sequences have a higher melting temperature (T_{M}) than do shorter ones. Oligonucleotide sequences with a high G-C content or that comprise palindromic sequences tend to self-hybridize, as do their intended target oligonucleotides, since unimolecular, rather than bimolecular, hybridization kinetics are generally favored in solution. However, it is also important to design an oligonucleotide that contains sufficient numbers of G-C nucleotide pairings since each G-C pair is bound by three hydrogen bonds, rather than the two that are found when A and T bases pair to bind the target sequence, and therefore forms a tighter, stronger bond. Hybridization temperature varies inversely with primer annealing efficiency, as does the concentration of organic solvents, e.g. formamide, that might be included in a hybridization mixture, while increases in salt concentration facilitate binding. Under stringent annealing conditions, longer hybridization oligonucleotides hybridize more efficiently than do shorter ones, which are sufficient under more permissive conditions. Stringent hybridization conditions typically include salt concentrations of less than about 1M, more usually less than about 500 mM and preferably less than about 200 mM, i.e., 100 mM. Hybridization temperatures range from as low as 0° C. to greater than 22° C., greater than about 30° C., and (most often) in excess of about 37° C., i.e., 68° C. Longer fragments may require higher hybridization temperatures for specific hybridization. As several factors affect the stringency of hybridization, the combination of parameters is more important than the absolute measure of a single factor.

Oligonucleotides can be designed with these considerations in mind and synthesized according to the following methods.

The design of a particular oligonucleotide for the purpose of forming an adaptor according to the invention involves selecting a sequence that is capable of annealing to a complementary oligonucleotide sequence, but has a minimal predicted secondary structure. The Tm of the oligonucleotide is optimized by analysis of the length and GC content of the oligonucleotide.

The design of an oligonucleotide is facilitated by the use of readily available computer programs, developed to assist in the evaluation of the several parameters described above and the optimization of oligonucleotide sequences. Examples of such programs are “PrimerSelect” of the DNAStar™ software package (DNAStar, Inc.; Madison, Wis.), OLIGO 4.0 (National Biosciences, Inc.), PRIMER, Oligonucleotide Selection Program, PGEN and Amplify (described in Ausubel et al., 1995, Short Protocols in Molecular Biology, 3rd Edition, John Wiley & Sons).

B. Oligonucleotide Synthesis

The oligonucleotides themselves are synthesized using techniques which are also well known in the art. Once designed, oligonucleotides are prepared by a suitable method, e.g. the phosphoramidite method described by Beaucage and Carruthers (1981, Tetrahedron Lett., 22:1859) or the triester method according to Matteucci et al. (1981, J. Am. Chem. Soc., 103:3185), both incorporated herein by reference, or by other chemical methods using either a commercial automated oligonucleotide synthesizer (which is commercially available) or VLSIPS™ technology.

C. Oligonucleotide Storage

Oligonucleotides according to the invention can be stored in solution at a concentration of 1-4 μg/μl, in TE buffer comprising 10 mM Tris (pH 7.5), 1 mM EDTA, at 4° C. and preferably at −20° C. Alternatively, an oligonucleotide can be stored in a lyophilized form wherein a solution of an oligonucleotide (1-4 μg/μl, in TE buffer) is dried down (i.e., in an eppendorf tube) for example in a speed-vac, according to methods well known in the art. The lyophilized oligonucleotide can be stored at room temperature, preferably at 4° C. and more preferably at −20° C.

An oligonucleotide according to the invention can be individually compartmentalized for storage, for example in an eppendorf tube, in a well of a plate (i.e., a 96 well plate), in a glass jar or a capillary tube or on a piece of filter paper.

III. Adaptors

A. How to Prepare Adaptors

The invention provides for adaptors comprising a double-stranded nucleic acid molecule formed by annealing two oligonucleotides. The adaptors according to the invention have a first and a second end, each of which are compatible with a nucleic acid digested with a restriction enzyme or with a nucleic acid comprising an end that is compatible with a nucleic acid digested with a restriction enzyme.

An adaptor is formed by preparing two complementary oligonucleotides as described above in the section entitled “Oligonucleotides” and annealing the two oligonucleotides to each other. Annealing is preferably carried out by mixing 1 μg/ml of one oligonucleotide and an equimolar amount of the second oligonucleotide. For example, a 12-mer oligonucleotide and a 16-mer oligonucleotide are annealed as follows:

A mix comprising

25 μl Oligo 1

33 μl Oligo 2 [=25 μl (Oligo 1)×16/12]

10 μl 10×MRB

32 μl H₂O

100 μl=Total (Final concentration is 0.58 μg/ul) is prepared in an eppendorf tube and placed in a rack in a 68° C. water bath for 10 minutes. After 10 minutes at 68° C., the adaptors are slow cooled to room temperature (ca. 22° C.). A portion of the stock of adaptor is kept frozen. A dilution comprising a working concentration of 1 ng/μl in 1× MRB (e.g. for the above example, add 2 μl of adaptor to 50 μl 10× MRB+450 μl H₂O) is also prepared and stored at 4° C. or preferably −20° C.

MRB is prepared as follows:

| 10X Medium Restriction Buffer (10XMRB) | 10 ml of 10X MRB |
|---|---|
| 100 mM Tris-Cl (pH 7.5) | 1 ml 1 M Tris (pH 7.5) |
| 500 mM NaCl | 1 ml 5 M NaCL |
| 100 mM MgCl | 1 ml 1 M MgCl₂ |
| 10 mM DTT | 100 μl 1 M DTT |

In certain embodiments, an adaptor according to the invention is phosphorylated. Phosphorylated adaptors are required if more than one adaptor is used in a ligation reaction. Because of nicks left by unphosphorylated adaptors following a ligation reaction, this linkage is thermodynamically unstable. If only one such double stranded nick exists in a nucleic acid molecule, this molecule can recircularize readily and enter bacteria where it is repaired, thus yielding transformants. However, two such nicks, as would be generated by the use of two unphosphorylated adaptors, would allow a fragment to separate from the plasmid molecule making bacterial transformation impossible.

Unphosphorylated adaptors are preferred if only one adaptor is required for a ligation reaction. By using an unphosphorylated adaptor in this setting, an excess of adaptor relative to vector and insert(s) can be used without generating multiple concatomeric adaptors in the cloning product.

Phosphorylation is carried out by preparing the following mixture:

1 μl Adaptor (1-100 ng)

1.5 μl Polynucleotide Kinase Buffer (10×)

1 μl Polynucleotide Kinase

1 μl ATP (10 mM)

10.5 μl H₂O

15 μl Total

The mixture is incubated for 1 hour at 37° C. A kinase buffer useful according to the invention comprises 70 mM Tris-HCl (pH 7.6), 10 mM MgCl₂, 5 mM dithiothreitol.

B. Adaptor Storage

Adaptors according to the invention are preferably stored in solution at a concentration of 1 ng/μl in 1× MRB at 4° C. or preferably −20° C. Alternatively, an adaptor can be stored in a lyophilized form wherein a solution of an adaptor (1 ng/μl in 1× MRB) is dried down (i.e., in an eppendorf tube) for example in a speed-vac, according to methods well known in the art. The lyophilized adaptor can be stored at room temperature, preferably at 4° C. and more preferably at −20° C.

An adaptor according to the invention can be individually compartmentalized for storage, for example in an eppendorf tube, in a well of a plate (i.e., a 96 well plate), in a glass jar or a capillary tube or on a piece of filter paper.

C. Categories of Adaptors

The invention provides for adaptors comprising a first end and a second end wherein the first and second ends are compatible with a nucleic acid digested with a restriction enzyme, or with a nucleic acid comprising an end that is compatible with a nucleic acid digested with a restriction enzyme, wherein the enzyme is selected from the group consisting of: any restriction enzyme known in the art, a standard restriction enzyme, a frequent-cutting restriction enzyme, an infrequent-cutting restriction enzyme, an enzyme that generates a blunt end, an enzyme that generates a 5′ overhang, an enzyme that generates a 3′ overhang or an enzyme that digests the polylinker of many commonly used cloning vectors.

The invention provides for adaptors wherein the first and second ends are compatible with a nucleic acid digested with the same category of restriction enzyme (e.g., both the first and second end are compatible with a nucleic acid digested with a standard restriction enzyme). The invention also provides for adaptors wherein the first and second ends are compatible with a nucleic acid digested with two different categories of restriction enzyme (e.g., the first end is compatible with a nucleic acid digested with a standard restriction enzyme and the second end is compatible with a nucleic acid digested with an enzyme that generates a blunt end).

The invention also provides for adaptors further comprising at least one start signal or at least one stop signal, a nucleic acid sequence from a gene of interest, a nucleic acid sequence encoding any amino acid that maintains the appropriate reading frame or an amino acid encoding a tag.

Categories of restriction enzymes useful according to the invention are presented in Tables 1-9.

TABLE 1
Standard Restriction Enzymes

| Enzyme | Site | End |
|---|---|---|
| Aat II | GACGTC | 3′ |
| Acc65 I | GGTACC | 5′ |
| Ad I | AACGTT | 5′ |
| Afe I | AGCGCT | Blunt |
| Afl II | CTTAAG | 5′ |
| Age I | ACCGGT | 5′ |
| Ahd I | GACNNJNNNGTC | 3′ |
| ALwN I | CAGNNNCTG | 3′ |
| Apa I | GGGCCC | 3′ |
| ApaL I | GTGCAC | 5′ |
| Ase I | ATTAAT | 5′ |
| Ayr II | CCTAGG | 5′ |
| Bae I | (10/15)AC(N4)GTAPyC(12/7) | 3′ |
| BamHI | GGATCC | 5′ |
| Bbs I | GAAGAC | 5′ |
| Bcg I | CGANNNNNNTGC | 3′ |
| BciV I | GTATCC | 3′ |
| Bel I | TGATCA | 5′ |
| BfrB I | ATGCAT | Blunt |
| Bgl I | GCCNNNNNGGC | 3′ |
| Bgl II | AGATCT | 5′ |
| Blp I | GCTNAGC | 5′ |
| Bmr I | ACTGGG | 3′ |
| Bpm I | CTGGAG | 3′ |
| BsaB I | GATNNNNATC | Blunt |
| Bsa I | GGTCTC | 5′ |
| BsaX I | 9(N)ACNNNNNCTCC(N)10 | 3′ |
| BseR I | GAGGAG | 3′ |
| Bsg I | GTGCAG | 3′ |
| BsiW I | CGTACG | 5′ |
| BsmB I | CGTCTC | 5′ |
| Bsm I | GAATGC | 3′ |
| BspD I | ATCGAT | 5′ |
| BspE I | TCCGGA | 5′ |
| BspH I | TCATGA | 5′ |
| BspM I | ACCTGC | 5′ |
| BsrB I | CCGCTC | Blunt |
| BsrD I | GCAATG | 3′ |
| BsrG I | TGTACA | 5′ |
| BssH II | GCGCGC | 5′ |
| BssS I | CACGAG | 5′ |
| BstAP I | GCANNNNNNTGC | 3′ |
| BstB I | TTCGAA | 5′ |
| BstE II | GGTNACC | 5′ |
| BstX I | CCANNNNNNTGG | 3′ |
| BstZ17 I | GTATAC | Blunt |
| Bsu36 I | CCTNAGG | 5′ |
| Btr I | CACGTC | Blunt |
| Bts I | GCAGTG | 3′ |
| Cla I | ATCGAT | 5′ |
| Dra I | TTTAAA | Blunt |
| Dra III | CACNNNGTG | 3′ |
| Drd I | GACNNNNNNGTC | 3′ |
| Eae I | YGGCCR | 5′ |
| Eag I | CGGCCG | 5′ |
| Ear I | CTCTTC | 5′ |
| Eci I | GGCGGA | 3′ |
| EcoN I | CCTNNNNNAGG | 5′ |
| EcoR I | GAATTC | 5′ |
| EcoR V | GATATC | Blunt |
| Fsp I | TGCGCA | Blunt |
| Hind III | AAGCTT | 5′ |
| Hpa I | GTTAAC | Blunt |
| Kas I | GGCGCC | 5′ |
| Kpn I | GGTACC | 3′ |
| Mfe I | CAATTG | 5′ |
| Mlu I | ACGCGT | 5′ |
| Msc I | TGGCCA | Blunt |
| Nae I | GCCGGC | Blunt |
| Nar I | GGCGCC | 5′ |
| Neo I | CCATGG | 5′ |
| Nde I | CATATG | 5′ |
| NgoM IV | GCCGGC | 5′ |
| Nhe I | GCTAGC | 5′ |
| Nru I | TCGCGA | Blunt |
| Nsi I | ATGCAT | 3′ |
| PaeR7 I | CTCGAG | 5′ |
| Pci I | ACATGT | 5′ |
| PflF I | GACNNNGTC | 5′ |
| PflM I | CCANNNNNTGG | 3′ |
| Pml I | CACGTG | Blunt |
| PshA I | GACNNNNGTC | Blunt |
| Psi I | TTATAA | Blunt |
| PspOM I | GGGCCC | 5′ |
| Pst I | CTGCAG | 3′ |
| Pvu I | CGATCG | 3′ |
| Pvu II | CAGCTG | Blunt |
| Sac I | GAGCTC | 3′ |
| Sac II | CCGCGG | 3′ |
| Sal I | GTCGAC | 5′ |
| Sca I | AGTACT | Blunt |
| Sfo I | GGCGCC | Blunt |
| Sma I | CCCGGG | Blunt |
| SnaB I | TACGTA | Blunt |
| Spe I | ACTAGT | 5′ |
| Sph I | GCATGC | 3′ |
| Ssp I | AATATT | Blunt |
| Stu I | AGGCCT | Blunt |
| Til I | CTCGAG | 5′ |
| Tth111 I | GACNNNGTC | 5′ |
| Xba I | TCTAGA | 5′ |
| Xcm I | CCANNNNNNNNNTGG | 3′ |
| Xho I | CTCGAG | 5′ |
| Xma I | CCCGGG | 5′ |
| Xmn I | GAANNNNTTC | Blunt |

TABLE 2
Enzymes that Generate a Blunt End:

| Enzyme | Site | End |
|---|---|---|
| Afe I | AGCGCT | Blunt |
| Alu I | AGCT | Blunt |
| BfrB I | ATGCAT | Blunt |
| BsaA I | YACGTR | Blunt |
| BsaB I | GATNNNNATC | Blunt |
| BstZ17 I | GTATAC | Blunt |
| Cac8 I | GCNNGC | Blunt |
| Dpn I | GATC | Blunt |
| Dra I | TTTAAA | Blunt |
| EcoR V | GATATC | Blunt |
| Fsp I | TGCGCA | Blunt |
| Hae III | GGCC | Blunt |
| Hinc II | GTYRAC | Blunt |
| Hpa I | GTTAAC | Blunt |
| HpyCH4V | TGCA | Blunt |
| Mly I | GAGTC | Blunt |
| Msc I | TGGCCA | Blunt |
| Msl I | CAYNNNNRTG | Blunt |
| MspA1 I | CMGCKG | Blunt |
| Nae I | GCCGGC | Blunt |
| Nla IV | GGNNCC | Blunt |
| Nru I | TCGCGA | Blunt |
| Pme I | GTTTAAAC | Blunt |
| Pml I | CACGTG | Blunt |
| PshA I | GACNNNNGTC | Blunt |
| Psi I | TTATAA | Blunt |
| Pvu II | CAGCTG | Blunt |
| Rsa I | GTAC | Blunt |
| Sca I | AGTACT | Blunt |
| Sfo I | GGCGCC | Blunt |
| Sma I | CCCGGG | Blunt |
| Ssp I | AATATT | Blunt |
| Stu I | AGGCCT | Blunt |
| Swa I | ATTTAAAT | Blunt |
| Xmn I | GAANNNNTTC | Blunt |

TABLE 3
Frequent Cutting Restriction Enzymes

| Enzyme | Site | End |
|---|---|---|
| Ace I | GTMKAC | 5′ |
| Aci I | CCGC | 5′ |
| Afi III | ACRYGT | 5′ |
| Alu I | AGCT | Blunt |
| Alw I | GGATC | 5′ |
| Apo I | RAATTY | 5′ |
| Ava I | CYCGRG | 5′ |
| Ava II | GGWCC | 5′ |
| Ban I | GGYRCC | 5′ |
| Ban II | GRGCYC | 3′ |
| Bbv I | GCAGC | 5′ |
| BceA I | ACGGC(12/13) | 5′ |
| Bfa I | CTAG | 5′ |
| BsaA I | YACGTR | Blunt |
| BsaH I | GRCGYC | 5′ |
| BsaJ I | CCNNGG | 5′ |
| BsaW I | WCCGGW | 5′ |
| BsiE I | CGRYCG | 3′ |
| BsiHKA I | GWGCWC | 3′ |
| Bsl I | CCNNNNNNNGG | 3′ |
| BsmA I | GTCTC | 5′ |
| BsmF I | GGGAC | 5′ |
| BsoB I | CYCGRG | 5′ |
| Bsp1286 I | GDGCHC | 3′ |
| BsrF I | RCCGGY | 5′ |
| Bsr I | ACTGG | 3′ |
| BssK I | CCNGG | 5′ |
| BstF5 I | GGATGNN | 3′ |
| BstN I | CCWGG | 5′ |
| BstU I | CGCG | Blunt |
| BstY I | RGATCY | 5′ |
| Btg I | CCPuPyGG | 5′ |
| Dde I | CTNAG | 5′ |
| Dpn I | GATC | Blunt |
| Dpn II | GATC | 5′ |
| EcoO109 I | RGGNCCY | 5′ |
| Fau I | CCCGC | 5′ |
| Fnu4H I | GCNGC | 5′ |
| Fok I | GGATG | 5′ |
| Hae II | RGCGCY | 3′ |
| Hae III | GGCC | Blunt |
| Hga I | GACGC | 5′ |
| Hha I | GCGC | 3′ |
| Hinc II | GTYRAC | Blunt |
| Hinf I | GANTC | 5′ |
| HinP1 I | GCGC | 5′ |
| Hpa II | CCGG | 5′ |
| Hpy188 I | TCNGA | 3′ |
| Hpy188 III | TCNNGA | 5′ |
| Hpy99 I | ACNGT | 3′ |
| HpyCH4 III | ACNGT | 3′ |
| HpyCH4 IV | ACGT | 5′ |
| HpyCH4 V | TGCA | Blunt |
| Hph I | GGTGA | 3′ |
| Mbo I | GATC | 5′ |
| Mbo II | GAAGA | 3′ |
| Mly I | GAGTC | Blunt |
| Mnl I | CCTC | 3′ |
| Mse I | TTAA | 5′ |
| Msl I | CAYNNNNRTG | Blunt |
| MspAl I | CMGCKG | Blunt |
| Msp I | CCGG | 5′ |
| Mwo I | GCNNNNNNNGC | 3′ |
| Nci I | CCSGG | 5′ |
| Nla III | CATG | 3′ |
| Nla IV | GGNNCC | Blunt |
| Nsp I | RCATGY | 3′ |
| Ple I | GAGTC | 5′ |
| PpuM I | RGGWCCY | 5′ |
| PspG I | CCWGG | 5′ |
| Rsa I | GTAC | Blunt |
| Rsr II | CGGWCCG | 5′ |
| Sau3A I | GATC | 5′ |
| Sau96 I | GGNCC | 5′ |
| ScrF I | CCNGG | 5′ |
| SfaN I | GCATC | 5′ |
| Sfc I | CTRYAG | 5′ |
| Sml I | CTYRAG | 5′ |
| Sty I | CCWWGG | 5′ |
| Taq I | TCGA | 5′ |
| Tfi I | GAWTC | 5′ |
| Tse I | GCWGC | 5′ |
| Tsp45 I | GTSAC | 5′ |
| Tsp509 I | AATT | 5′ |
| TspR I | CAGTG | 3′ |

TABLE 4
Infrequent Cutting Restriction Enzymes

| | | |
|---|---|---|
| Asc I | GGCGCGCC | 5′ |
| BbvC I | CCTCAGC | 5′ |
| Fse I | GGCCGGCC | 3′ |
| Not I | GCGGCCGC | 5′ |
| Pac I | TTAATTAA | 3′ |
| Pme I | GTTTAAAC | Blunt |
| Sap I | GCTCTTC | 5′ |
| Sbf I | CCTGCAGG | 3′ |
| SexA I | ACCWGGT | 5′ |
| Sfi I | GGCCNNNNNGGCC | 3′ |
| SgrA I | CRCCGGYG | 5′ |
| Swa I | ATTTAAAT | Blunt |

TABLE 5
Enzymes that Generate 3′ Overhangs

| Enzyme | Site | End |
|---|---|---|
| Aat II | GACGTC | 3′ |
| Ahd I | GACNNNNGTC | 3′ |
| AlwN I | CAGNNNCTG | 3′ |
| Apa I | GGGCCC | 3′ |
| Bae I | (10/15)AC(N4)GTAPyC(12/7) | 3′ |
| Bcg I | CGANNNNNNTGC | 3′ |
| BciV I | GTATCC | 3′ |
| Bgl I | GCCNNNNNGGC | 3′ |
| Bmr I | ACTGGG | 3′ |
| Bpm I | CTGGAG | 3′ |
| BsaX I | 9(N)ACNNNNNNCTCC(N)10 | 3′ |
| BseR I | GAGGAG | 3′ |
| Bsg I | GTGCAG | 3′ |
| Bsm I | GAATGC | 3′ |
| BsrD I | GCAATG | 3′ |
| BstAP I | GCANNNNNTGC | 3′ |
| BstX I | CCANNNNNNTGG | 3′ |
| Bts I | GCAGTG | 3′ |
| Dra III | CACNNNGTG | 3′ |
| Drd I | GACNNNNNNGTC | 3′ |
| Eci I | GGCGGA | 3′ |
| Kpn I | GGTACC | 3′ |
| Nsi I | ATGCAT | 3′ |
| PflM I | CCANNNNNTGG | 3′ |
| Pst I | CTGCAG | 3′ |
| Pvu I | CGATCG | 3′ |
| Sac I | GAGCTC | 3′ |
| Sac II | CCGCGG | 3′ |
| Sph I | GCATGC | 3′ |
| Xcm I | CCANNNNNNNNNTGG | 3′ |
| Ban II | GRGCYC | 3′ |
| BsiE I | CGRYCG | 3′ |
| BsiHKA I | GWGCWC | 3′ |
| Bsl I | CCNNNNNNNGG | 3′ |
| Bsp1286 I | GDGCHC | 3′ |
| Bsr I | ACTGG | 3′ |
| BstF5 I | GGATGNN | 3′ |
| HaeII | RGCGCY | 3′ |
| Hha I | GCGC | 3′ |
| Hpy188 I | TCNGA | 3′ |
| Hpy99 I | ACNGT | 3′ |
| HpyCH4 III | ACNGT | 3′ |
| Hph I | GGTGA | 3′ |
| Mbo II | GAAGA | 3′ |
| Mnl I | CCTC | 3′ |
| Mwo I | GCNNNNNNNGC | 3′ |
| Nla III | CATG | 3′ |
| Nsp I | RCATGY | 3′ |
| TspR I | CAGTG | 3′ |
| Fse I | GGCCGGCC | 3′ |
| Pac I | TTAATTAA | 3′ |
| Sbf I | CCTGCAGG | 3′ |
| Sfi I | GGCCNNNNNGGCC | 3′ |
| SgrA I | CRCCGGYG | 5′ |

TABLE 6
Enzymes that Generate 5′ Overhangs

| Enzyme | Site | End |
|---|---|---|
| Acc65 I | GGTACC | 5′ |
| Acc I | GTMKAC | 5′ |
| Acl I | AACGTT | 5′ |
| Acl I | CCGC | 5′ |
| Afi II | CTTAAG | 5′ |
| Afi III | ACRYGT | 5′ |
| Age I | ACCGGT | 5′ |
| Alw I | GGATC | 5′ |
| ApaL I | GTGCAC | 5′ |
| Apo I | RAATTY | 5′ |
| Asc I | GGCGCGCC | 5′ |
| Ase I | ATTAAT | 5′ |
| Ava I | CYCGRG | 5′ |
| Ava II | GGWCC | 5′ |
| Avr II | CCTAGG | 5′ |
| Ban I | GGYRCC | 5′ |
| BamH I | GGATCC | 5′ |
| Bbs I | GAAGAC | 5′ |
| BbvC I | CCTCAGC | 5′ |
| Bbv I | GCAGC | 5′ |
| BceA I | ACGGC(12/13) | 5′ |
| Bcl I | TGATCA | 5′ |
| Bfa I | CTAG | 5′ |
| Bgl II | AGATCT | 5′ |
| Blp I | GCTNAGC | 5′ |
| BsaH I | GRCGYC | 5′ |
| Bsa I | GGTCTC | 5′ |
| BsaJ I | CCNNGG | 5′ |
| BsaW I | WCCGGW | 5′ |
| BsiW I | CGTACG | 5′ |
| BsmA I | GTCTC | 5′ |
| BsmB I | CGTCTC | 5′ |
| BsmF I | GGGAC | 5′ |
| BspD I | ATCGAT | 5′ |
| BspE I | TCCGGA | 5′ |
| BspH I | TCATGA | 5′ |
| BspM I | ACCTGC | 5′ |
| BsoB I | CYCGRG | 5′ |
| BsrF I | RCCGGY | 5′ |
| BsrG I | TGTACA | 5′ |
| BssH II | GCGCGC | 5′ |
| BssK I | CCNGG | 5′ |
| BssS I | CACGAG | 5′ |
| BstB I | TTCGAA | 5′ |
| BstE II | GGTNACC | 5′ |
| BstN I | CCWGG | 5′ |
| BstY I | RGATCY | 5′ |
| Bsu36 I | CCTNAGG | 5′ |
| Btg I | CCPuPyGG | 5′ |
| Cla I | ATCGAT | 5′ |
| Dde I | CTNAG | 5′ |
| Dpn II | GATC | 5′ |
| Eae I | YGGCCR | 5′ |
| Eag I | CGGCCG | 5′ |
| Ear I | CTCTTC | 5′ |
| EcoN I | CCTNNNNNAGG | 5′ |
| EcoO109 I | RGGNCCY | 5′ |
| EcoR I | GAATTC | 5′ |
| Fau I | CCCGC | 5′ |
| Fnu4H I | GCNGC | 5′ |
| Fok I | GGATG | 5′ |
| Hga I | GACGC | 5′ |
| Hind III | AAGCTT | 5′ |
| Hinf I | GANTC | 5′ |
| HinPl I | GCGC | 5′ |
| Hpa II | CCGG | 5′ |
| Hpy188 III | TCNNGA | 5′ |
| HpyCH4IV | ACGT | 5′ |
| Kas I | GGCGCC | 5′ |
| Mbo I | GATC | 5′ |
| Mfe I | CAATTG | 5′ |
| Mlu I | ACGCGT | 5′ |
| Mse I | TTAA | 5′ |
| Msp I | CCGG | 5′ |
| Nar I | GGCGCC | 5′ |
| Nci I | CCSGG | 5′ |
| Nco I | CCATGG | 5′ |
| Nde I | CATATG | 5′ |
| NgoM IV | GCCGGC | 5′ |
| Nhe I | GCTAGC | 5′ |
| Not I | GCGGCCGC | 5′ |
| PaeR7 I | CTCGAG | 5′ |
| Pci I | ACATGT | 5′ |
| PflF I | GACNNNGTC | 5′ |
| Ple I | GAGTC | 5′ |
| PpuM I | RGGWCCY | 5′ |
| PspG I | CCWGG | 5′ |
| PspOM I | GGGCCC | 5′ |
| Rsr II | CGGWCCG | 5′ |
| Sal I | GTCGAC | 5′ |
| Sap I | GCTCTTC | 5′ |
| Sau96 I | GGNCC | 5′ |
| Sau3A I | GATC | 5′ |
| ScrF I | CCNGG | 5′ |
| SexA I | ACCWGGT | 5′ |
| SfaN I | GCATC | 5′ |
| Sfc I | CTRYAG | 5′ |
| SgrA I | CRCCGGYG | 5′ |
| Sml I | CTYRAG | 5′ |
| Spe I | ACTAGT | 5′ |
| Sty I | CCWWGG | 5′ |
| Taq I | TCGA | 5′ |
| Tfi I | GAWTC | 5′ |
| Tli I | CTCGAG | 5′ |
| Tse I | GCWGC | 5′ |
| Tsp45 I | GTSAC | 5′ |
| Tsp509 I | AATT | 5′ |
| Tth111 I | GACNNNGTC | 5′ |
| Xba I | TCTAGA | 5′ |
| Xho I | CTCGAG | 5′ |
| Xma I | CCCGGG | 5′ |

TABLE 7
Enzymes capable of digesting common cloning vector
polylinkers

| Enzyme | Site | End |
|---|---|---|
| Aat II | GACGTC | 3′ |
| Apa I | GGGCCC | 3′ |
| Ava I | CYCGRG | 5′ |
| BamH I | GGATCC | 5′ |
| Bgl II | AGATCT | 5′ |
| BstX I | CCANNNNNNTGG | 3′ |
| Cla I | ATCGAT | 5′ |
| Eag I | CGGCCG | 5′ |
| EcoR I | GAATTC | 5′ |
| EcoR V | GATATC | Blunt |
| Hinc II | GTYRAC | Blunt |
| Hind III | AAGCTT | 5′ |
| Kpn I | GGTACC | 3′ |
| Mlu I | ACGCGT | 5′ |
| Nco I | CCATGG | 5′ |
| Nde I | CATATG | 5′ |
| Nhe I | GCTAGC | 5′ |
| Not I | GCGGCCGC | 5′ |
| Nsi I | ATGCAT | 3′ |
| Pac I | TTAATTAA | 3′ |
| Pme I | GTTTAAAC | Blunt |
| Pst I | CTGCAG | 3′ |
| Pvu II | CAGCTG | Blunt |
| Sac I | GAGCTC | 3′ |
| Sac II | CCGCGG | 3′ |
| Sal I | GTCGAC | 5′ |
| Sfi I | GGCCNNNNNGGCC | 3′ |
| Sma I | CCCGGG | Blunt |
| Sph I | GCATGC | 3′ |
| Xba I | TCTAGA | 5′ |
| Xho I | CTCGAG | 5′ |
| Xma I | CCCGGG | 5′ |

TABLE 8
Restriction Enzymes that generate sticky ends

| | | |
|---|---|---|
| Aat II | GACGTC | 3′ |
| Acc65 I | GGTACC | 5′ |
| Acc I | GTMKAC | 5′ |
| Aci I | CCGC | 5′ |
| Acl I | AACGTT | 5′ |
| Afl II | CTTAAG | 5′ |
| Afl III | ACRYGT | 5′ |
| Age I | ACCGGT | 5′ |
| Ahd I | GACNNNNNGTC | 3′ |
| Alw I | GGATC | 5′ |
| AlwN I | CAGNNNCTG | 3′ |
| Apa I | GGGCCC | 3′ |
| ApaL I | GTGCAC | 5′ |
| Apo I | RAATTY | 5′ |
| Asc I | GGCGCGCC | 5′ |
| Ase I | ATTAAT | 5′ |
| Ava I | CYCGRG | 5′ |
| Ava II | GGWCC | 5′ |
| Avr II | CCTAGG | 5′ |
| Bae I | (10/15)AC(N4)GTAPyC(12/7) | 3′ |
| BamH I | GGATCC | 5′ |
| Ban I | GGYRCC | 5′ |
| Ban II | GRGCYC | 3′ |
| Bbs I | GAAGAC | 5′ |
| BbvC I | CCTCAGC | 5′ |
| Bbv I | GCAGC | 5′ |
| BceA I | ACGGC(12/13) | 5′ |
| Bcg I | CGANNNNNNTGC | 3′ |
| BciV I | GTATCC | 3′ |
| Bcl I | TGATCA | 5′ |
| Bfa I | CTAG | 5′ |
| Bgl I | GCCNNNNNGGC | 3′ |
| Bgl II | AGATCT | 5′ |
| Blp I | GCTNAGC | 5′ |
| Bmr I | ACTGGG | 3′ |
| Bpm I | CTGGAG | 3′ |
| BsaH I | GRCGYC | 5′ |
| Bsa I | GGTCTC | 5′ |
| BsaJ I | CCNNGG | 5′ |
| BsaW I | WCCGGW | 5′ |
| BsaX I | 9(N)ACNNNNNCTCC(N)10 | 3′ |
| BseR I | GAGGAG | 3′ |
| Bsg I | GTGCAG | 3′ |
| BsiE I | CGRYCG | 3′ |
| BsiHKA I | GWGCWC | 3′ |
| BsiW I | CGTACG | 5′ |
| Bsl I | CCNNNNNNNGG | 3′ |
| BsmA I | GTCTC | 5′ |
| BsmB I | CGTCTC | 5′ |
| BsmF I | GGGAC | 5′ |
| Bsm I | GAATGC | 3′ |
| BsoB I | CYCGRG | 5′ |
| Bsp1286 I | GDGCHC | 3′ |
| BspD I | ATCGAT | 5′ |
| BspE I | TCCGGA | 5′ |
| BspH I | TCATGA | 5′ |
| BspM I | ACCTGC | 5′ |
| BsrD I | GCAATG | 3′ |
| BsrF I | RCCGGY | 5′ |
| BsrG I | TGTACA | 5′ |
| Bsr I | ACTGG | 3′ |
| BssH II | GCGCGC | 5′ |
| BssK I | CCNGG | 5′ |
| BssS I | CACGAG | 5′ |
| BstAP I | GCANNNNNTGC | 3′ |
| BstB I | TTCGAA | 5′ |
| BstE II | GGTNACC | 5′ |
| BstF5 I | GGATGNN | 3′ |
| BstN I | CCWGG | 5′ |
| BstX I | CCANNNNNNTGG | 3′ |
| BstY I | RGATCY | 5′ |
| Bsu36 I | CCTNAGG | 5′ |
| Btg I | CCPuPyGG | 5′ |
| Bts I | GCAGTG | 3′ |
| Cla I | ATCGAT | 5′ |
| Dde I | CTNAG | 5′ |
| Dpn II | GATC | 5′ |
| Dra III | CACNNNGTG | 3′ |
| Drd I | GACNNNNNNGTC | 3′ |
| Eae I | YGGCCR | 5′ |
| Eag I | CGGCCG | 5′ |
| Ear I | CTCTTC | 5′ |
| Eci I | GGCGGA | 3′ |
| EcoN I | CCTNNNNTNAGG | 5′ |
| EcoO109 I | RGGNCCY | 5′ |
| EcoR I | GAATTC | 5′ |
| Fau I | CCCGC | 5′ |
| Fnu4H I | GCNGC | 5′ |
| Fok I | GGATG | 5′ |
| Fse I | GGCCGGCC | 3′ |
| Hae II | RGCGCY | 3′ |
| Hga I | GACGC | 5′ |
| Hha I | GCGC | 3′ |
| Hind III | AAGCTT | 5′ |
| Hinf I | GANTC | 5′ |
| HinPl I | GCGC | 5′ |
| Hpa II | CCGG | 5′ |
| Hpy188 I | TCNGA | 3′ |
| Hpy188 III | TCNNGA | 5′ |
| Hpy99 I | ACNGT | 3′ |
| HpyCH4 III | ACNGT | 3′ |
| HpyCH4 IV | ACGT | 5′ |
| Hph I | GGTGA | 3′ |
| Kas I | GGCGCC | 5′ |
| Kpn I | GGTACC | 3′ |
| Mbo I | GATC | 5′ |
| Mbo II | GAAGA | 3′ |
| Mfe I | CAATTG | 5′ |
| Mlu I | ACGCGT | 5′ |
| Mnl I | CCTC | 3′ |
| Mse I | TTAA | 5′ |
| Msp I | CCGG | 5′ |
| Mwo I | GCNNNNNNNGC | 3′ |
| Nar I | GGCGCC | 5′ |
| Nci I | CCSGG | 5′ |
| Nco I | CCATGG | 5′ |
| Nde I | CATATG | 5′ |
| NgoM IV | GCCGGC | 5′ |
| Nhe I | GCTAGC | 5′ |
| Nla III | CATG | 3′ |
| Not I | GCGGCCGC | 5′ |
| Nsi I | ATGCAT | 3′ |
| Nsp I | RCATGY | 3′ |
| Pac I | TTAATTAA | 3′ |
| PaeR7 I | CTCGAG | 5′ |
| Pci I | ACATGT | 5′ |
| PflF I | GACNNNGTC | 5′ |
| PflM I | CCANNNNNTGG | 3′ |
| Ple I | GAGTC | 5′ |
| PpuM I | RGGWCCY | 5′ |
| PspG I | CCWGG | 5′ |
| PspOM I | GGGCCC | 5′ |
| Pst I | CTGCAG | 3′ |
| Pvu I | CGATCG | 3′ |
| Rsr II | CGGWCCG | 5′ |
| Sac I | GAGCTC | 3′ |
| Sac II | CCGCGG | 3′ |
| Sal I | GTCGAC | 5′ |
| Sap I | GCTCTTC | 5′ |
| Sau3A I | GATC | 5′ |
| Sau96 I | GGNCC | 5′ |
| Sbf I | CCTGCAGG | 3′ |
| ScrF I | CCNGG | 5′ |
| SexA I | ACCWGGT | 5′ |
| SfaN I | GCATC | 5′ |
| Sfc I | CTRYAG | 5′ |
| Sfi I | GGCCNNNNNGGCC | 3′ |
| SgrA I | CRCCGGYG | 5′ |
| Sml I | CTYRAG | 5′ |
| Spe I | ACTAGT | 5′ |
| Sph I | GCATGC | 3′ |
| Sty I | CCWWGG | 5′ |
| Taq I | TCGA | 5′ |
| Tfi I | GAWTC | 5′ |
| Tli I | CTCGAG | 5′ |
| Tse I | GCWGC | 5′ |
| Tsp45 I | GTSAC | 5′ |
| Tsp509 I | AATT | 5′ |
| TspR I | CAGTG | 3′ |
| Tth111 I | GACNNNGTC | 5′ |
| Xba I | TCTAGA | 5′ |
| Xcm I | CCANNNNNNNNNTGG | 3′ |
| Xho I | CTCGAG | 5′ |
| Xma I | CCCGGG | 5′ |

TABLE 9
Restriction Enzymes

| Enzyme | Site | End |
|---|---|---|
| Aat II | GACGTC | 3′ |
| Acc65 I | GGTACC | 5′ |
| Acc I | GTMKAC | 5′ |
| Aci I | CCGC | 5′ |
| Acl I | AACGTT | 5′ |
| Afe I | AGCGCT | Blunt |
| Afl II | CTTAAG | 5′ |
| Afl III | ACRYGT | 5′ |
| Age I | ACCGGT | 5′ |
| Ahd I | GACNNNNNGTC | 3′ |
| Alu I | AGCT | Blunt |
| Alw I | GGATC | 5′ |
| AlwN I | CAGNNNCTG | 3′ |
| Apa I | GGGCCC | 3′ |
| ApaL I | GTGCAC | 5′ |
| Apo I | RAATTY | 5′ |
| Asc I | GGCGCGCC | 5′ |
| Ase I | ATTAAT | 5′ |
| Ava I | CYCGRG | 5′ |
| Ava II | GGWCC | 5′ |
| Avr II | CCTAGG | 5′ |
| Bae I | (10/15)AC(N4)GTAPyC(12/7) | 3′ |
| BamH I | GGATCC | 5′ |
| Ban I | GGYRCC | 5′ |
| Ban II | GRGCYC | 3′ |
| Bbs I | GAAGAC | 5′ |
| BbvC I | CCTCAGC | 5′ |
| Bbv I | GCAGC | 5′ |
| BceA I | ACGGC(12/13) | 5′ |
| Bcg I | CGANNNNNNTGC | 3′ |
| BciV I | GTATCC | 3′ |
| Bcl I | TGATCA | 5′ |
| Bfa I | CTAG | 5′ |
| BfrB I | ATGCAT | Blunt |
| Bgl I | GCCNNNNNGGC | 3′ |
| Bgl II | AGATCT | 5′ |
| Blp I | GCTNAGC | 5′ |
| Bmr I | ACTGGG | 3′ |
| Bpm I | CTGGAG | 3′ |
| BsaB I | GATNNNNATC | Blunt |
| BsaA I | YACGTR | Blunt |
| BsaH I | GRCGYC | 5′ |
| Bsa I | GGTCTC | 5′ |
| BsaJ I | CCNNGG | 5′ |
| BsaW I | WCCGGW | 5′ |
| BsaX I | 9(N)ACNNNNNCTCC(N)10 | 3′ |
| BseR I | GAGGAG | 3′ |
| Bsg I | GTGCAG | 3′ |
| BsiE I | CGRYCG | 3′ |
| BsiHKA I | GWGCWC | 3′ |
| BsiW I | CGTACG | 5′ |
| Bsl I | CCNNNNNNNGG | 3′ |
| BsmA I | GTCTC | 5′ |
| BsmB I | CGTCTC | 5′ |
| BsmF I | GGGAC | 5′ |
| Bsm I | GAATGC | 3′ |
| BsoB I | CYCGRG | 5′ |
| Bsp1286 I | GDGCHC | 3′ |
| BspD I | ATCGAT | 5′ |
| BspE I | TCCGGA | 5′ |
| BspH I | TCATGA | 5′ |
| BspM I | ACCTGC | 5′ |
| BsrB I | CCGCTC | Blunt |
| BsrD I | GCAATG | 3′ |
| BsrF I | RCCGGY | 5′ |
| BsrG I | TGTACA | 5′ |
| Bsr I | ACTGG | 3′ |
| BssH II | GCGCGC | 5′ |
| BssK I | CCNGG | 5′ |
| BssS I | CACGAG | 5′ |
| BstAP I | GCANNNNNTGC | 3′ |
| BstB I | TTCGAA | 5′ |
| BstE II | GGTNACC | 5′ |
| BstF5 I | GGATGNN | 3′ |
| BstN I | CCWGG | 5′ |
| BstU I | CGCG | Blunt |
| BstX I | CCANNNNNTGG | 3′ |
| BstY I | RGATCY | 5′ |
| BstZ17 I | GTATAC | Blunt |
| Bsu36 I | CCTNAGG | 5′ |
| Btg I | CCPuPyGG | 5′ |
| Btr I | CACGTC | Blunt |
| Bts I | GCAGTG | 3′ |
| Cac8 I | GCNNGC | Blunt |
| Cla I | ATCGAT | 5′ |
| Dde I | CTNAG | 5′ |
| Dpn I | GATC | Blunt |
| Dpn II | GATC | 5′ |
| Dra I | TTTAAA | Blunt |
| Dra III | CACNNNGTG | 3′ |
| Drd I | GACNNNNNNGTC | 3′ |
| Eae I | YGGCCR | 5′ |
| Eag I | CGGCCG | 5′ |
| Ear I | CTCTTC | 5′ |
| Eci I | GGCGGA | 3′ |
| EcoN I | CCTNNNNNAGG | 5′ |
| EcoO109 I | RGGNCCY | 5′ |
| EcoR I | GAATTC | 5′ |
| EcoR V | GATATC | Blunt |
| Fau I | CCCGC | 5′ |
| Fnu4H I | GCNGC | 5′ |
| Fok I | GGATG | 5′ |
| Fse I | GGCCGGCC | 3′ |
| Fsp I | TGCGCA | Blunt |
| Hae II | RGCGCY | 3′ |
| Hae III | GGCC | Blunt |
| Hga I | GACGC | 5′ |
| Hha I | GCGC | |
| Hinc II | GTYRAC | Blunt |
| Hind III | AAGCTT | 5′ |
| Hinf I | GANTC | 5′ |
| HinP1 I | GCGC | 5′ |
| Hpa I | GTTAAC | Blunt |
| Hpa II | CCGG | 5′ |
| Hpy188 I | TCNGA | 3′ |
| Hpy188 III | TCNNGA | 5′ |
| Hpy99 I | ACNGT | 3′ |
| HpyCH4 III | ACNGT | 3′ |
| HpyCH4 IV | ACGT | 5′ |
| HpyCH4 V | TGCA | Blunt |
| Hph I | GGTGA | 3′ |
| Kas I | GGCGCC | 5′ |
| Kpn I | GGTACC | 3′ |
| Mbo I | GATC | 5′ |
| Mbo II | GAAGA | 3′ |
| Mfe I | CAATTG | 5′ |
| Mlu I | ACGCGT | 5′ |
| Mly I | GAGTC | Blunt |
| Mnl I | CCTC | 3′ |
| Msc I | TGGCCA | Blunt |
| Mse I | TTAA | 5′ |
| Msl I | CAYNNNNRTG | Blunt |
| MspA1 I | CMGCKG | Blunt |
| Msp I | CCGG | 5′ |
| Mwo I | GCNNNNNNNGC | 3′ |
| Nae I | GCCGGC | Blunt |
| Nan | GGCGCC | 5′ |
| Nci I | CCSGG | 5′ |
| Nco I | CCATGG | 5′ |
| Nde I | CATATG | 5′ |
| NgoM IV | GCCGGC | 5′ |
| Nhe I | GCTAGC | 5′ |
| Nla III | CATG | 3′ |
| Nla IV | GGNNCC | Blunt |
| Not I | GCGGCCGC | 5′ |
| Nru I | TCGCGA | Blunt |
| Nsi I | ATGCAT | 3′ |
| Nsp I | RCATGY | 3′ |
| Pac I | TTAATTAA | 3′ |
| PaeR7 I | CTCGAG | 5′ |
| Pci I | ACATGT | 5′ |
| PflF I | GACNNNGTC | 5′ |
| PflM I | CCANNNNNTGG | 3′ |
| Ple I | GAGTC | 5′ |
| Pme I | GTTTAAAC | Blunt |
| Pml I | CACGTG | Blunt |
| PpuM I | RGGWCCY | 5′ |
| PshA I | GACNNNNGTC | Blunt |
| Psi I | TTATAA | Blunt |
| PspG I | CCWGG | 5′ |
| PspOM I | GGGCCC | 5′ |
| Pst I | CTGCAG | 3′ |
| Pvu I | CGATCG | 3′ |
| Pvu II | CAGCTG | Blunt |
| Rsa I | GTAC | Blunt |
| Rsr II | CGGWCCG | 5′ |
| Sal | GAGCTC | 3′ |
| Sac II | CCGCGG | 3′ |
| Sal I | GTCGAC | 5′ |
| Sap I | GCTCTTC | 5′ |
| Sau3A I | GATC | 5′ |
| Sau96 I | GGNCC | 5′ |
| Sbf I | CCTGCAGG | 3′ |
| Sca I | AGTACT | Blunt |
| SerF I | CCNGG | 5′ |
| SexA I | ACCWGGT | 5′ |
| SfaN I | GCATC | 5′ |
| Sfc I | CTRYAG | 5′ |
| Sfi I | GGCCNNNNNGGCC | 3′ |
| Sfo I | GGCGCC | Blunt |
| SgrA I | CRCCGGYG | 5′ |
| Sma I | CCCGGG | Blunt |
| Sml I | CTYRAG | 5′ |
| SnaB I | TACGTA | Blunt |
| Spe I | ACTAGT | 5′ |
| Sph I | GCATGC | 3′ |
| Ssp I | AATATT | Blunt |
| Stu I | AGGCCT | Blunt |
| Sty I | CCWWGG | 5′ |
| Swa I | ATTTAAAT | Blunt |
| Taq I | TCGA | 5′ |
| Tfi I | GAWTC | 5′ |
| Tli I | CTCGAG | 5′ |
| Tse I | GCWGC | 5′ |
| Tsp45 I | GTSAC | 5′ |
| Tsp509 I | AATT | 5′ |
| TspR I | CAGTG | 3′ |
| Tth111 I | GACNNNGTC | 5′ |
| Xba I | TCTAGA | 5′ |
| Xcm I | CCANNNNNNNNNTGG | 3′ |
| Xho I | CTCGAG | 5′ |
| Xma I | CCCGGG | 5′ |
| Xmn I | GAANNNNTTC | Blunt |

TABLE 10
Commody Used Cloning Vectors

pBR322
pUC18
pUC19
A Promega
pGEM-3Z
pGEM-4Z
pGEM-3Zf(+/−)
pGEM-5Zf(+/−)
pGEM-7Zf(+/−)
pGEM-9Zf(−)
pGEM-11Zf(+/−)
pGEM-13Zf(+)
pSP72
pSP73
pSP64 Poly(A)
pGEMEX-1
pGEMEX-2
pET-5a
pET-9a
pALTER-1
pALTER-Ex1
pALTER-Ex2
Lambda gt11
LambdaGEM-11
LambdaGEM-12
EMBL3
EMBL4
pGL3-Basic
pGL3-Enhancer
pGL3 -Promoter
pGL3 -Control
pRL-SV40
pRL-TK
pRL-CMV
pRE-null
pTargeT
pSI
pCI
B. In Vitrogen
pBAD/His
pBAD/Myc—His
pBAD/gIII
pTrxFus
pThioHis
pRSET
pTrcHis-TOPO
pTrcHis
pSE280
pSE380
pSE420
pLEX
pcDNA2.1
pPICZ
pGAPZ
pPIC9K
pPic3.5K
pAO815
pMET
YES
pYES2.1/VS
pYD1
pTEF1/Zeo
pTEF1Bsd
pIZT/V5-His
pIZ/V5-His
pIZT/V5-His
pIB/V5-His
pIB/V5-His-TOPO
pMT/V5-His
pMT/BiP/V5-His
pAc5.1/V5-His
pDS47/V5-His
pBlueBac4.5/V5-His-TOPO
pBlueBac4.5/V5-His
pBlueBac4.5
pBlueBacHis2
pMe1Bac
pVL1392
pIND
pVgRXR
pcDNA4/TO
pcDNA4/TO/myc—his
pcDNA6/TR
oGene/V5-His
EpiTag/His
EpiTag/myc—His
EpiTag/V5-His
pcDNA3.1
pcDNA3.1/Zeo
pcDNA3.1/Hygro
pcDNA4/HisMax
pBudCE4
pVAX1
pRc/CMV2
pRc/RSV
pZeoSV2
pEF6/V5-His-TOPO
pcDNA3.1/V5-His-TOPO
pCR3.1
pSecTag2
pDisplaypCDM8
pcDNA1.1
pcDNA1.1/Amp
pFRT/lacZeo
pcDNA5/FRT
pHook-1
pEF/myc/nuc
pCMV/myc/nuc
pVP22
pREP4
pREP8
pREP9
pCEP4
pREP7
pREP10
pEBVHis
pCMV/Zeo
pSV40/Zeo
pEM7/Zeo
pTEF1/Zeo
pEM7/Bsd
pTEF1/Bsd
pCMV/Bsd
pBlue-TOPO
pGlow-TOPO
pTracer-SV40
pTracer-CMV2
pTracer-EF
pTracer-CMV/Bsd
pcDNA3.1/GS
pIND/GS
pCR-T7/GS
pBAD/ThioGS
ocDNA3.1/GS
pYES2/GS
C. Life Technologies
pBS185
pBS246
pBS302
pCMV-SPORT-beta-gal
pFastBAc1
pFastBac HT
pFastBac DUAL
pSPORT1
D. New England Biolabs
pLJTMUS
pMAL
pTYB
p193
M13mp
E. Stratagene
pCMV-Script
pCMV-Tag
pXT1
pSG5
pPbac
pMbac
pDual
pCMVLacI
pOPRSV/MCS
pOPI3Cat
pESC
pESP-1
pESP-2
pESP-3
pCAL
pET-3
pET-11
pSPUTK
pOG44
pOG45
pFRTbetaGAL
pNIEObetaGAL
pMClneo
pCMV-Tag
pRS
pBK-CMV
pCMV-Script-EX
pBluescript
pBluescript II
SuperCos
pWE15
pPCR-Script Amp
pPCR-Script Cam
BC
F. Clontech
pAdeno
pShuttle
TRE Shuttle
pDNR-1
pDNR-2
pDNR-3
pDNR-d2EGFP
pDNR-EGFP
pDNR-LIB
pDNR-LacZ
pDNR-SEAP
pIRES
pIRES-EGFP
pIRES-EYFP
pIRBS2-EGFP
pIRESbleo
pIREShyg
pIREShyg2
pIRESneo
pIRESneo2
plRESpuro
pIRIESpuro2
pDsRed
pDsRed1-1
pDsRed1-C1
pDsRed1-N1
pDsRed2
pDsRed2-1
pDsRed2-C1
pDsRed2-N1
pTimer-1
pBI-EGFP
pCMS-EGFP
pCRE-d2EGFP
pCRBB-EGFP
pd1EGFP-N1
pd2EGFP
pd2EGFP-1
pd2EGFP-N1
pd4EGFP-N1
pEGFP
pEGFP-1
pEGFP-C1
pEGFP-C2
pEGFP-C3
pEGFP-N1
pEGFP-N2
pEGFP-N3
pEGFPLuc
pGRE-d2EGFP
pHygEGFP
pIkB-EGFP
pIRBS2-EGFP
pNFkB-d2EGFP
pPKCb-EGFP
pPKCg-EGFP
pTAL-d2EGFP
pTRE-d2EGFP
pd2ECFP-1
pd2ECFP-N1
pECFP
pECFP-1
pECFP-C1
pECFP-N1
pd2EYFP-1
pd2EYFP-N1
pEYFP
pEYFP-1
pEYFP-C1
pEYFP-N1
pIRES-EYFP
pEBFP
pEBFP-C 1
pEBFP-N1 (d)
pLEGFP-C1
pLEGFP-N1
pDsRed1-Mito
pEGFP-Actin
pEYFP-Actin
pECFP-Endo
pEGFP-Endo
pEYFP-Endo
pECFP-ER
pEYFP-ER
pEGFP-F
pECFP-Golgi
pEYFP-Golgi
pECFP-Mem
pEYFP-Mem
pECFP-Mito
pEYFP-Mito
pECFP-Nuc
pEYFP-Nuc
pECFP-Peroxi
pEGFP-Peroxi
pEYFP-Peroxi
pEGFP-Tub
pEYFP-Tub
pBFP2
pGFP
pGFP(AAV)
pGFP(ASV)
pGFP(LVA)
pGFPmut3.1
pGFPuv
p35S-GFP
pBAD-GFPuv
pd2EGFP-Basic
pd2EGFP-Control
pd2EGFP- Enhancer
pd2EGFP-Promoter
pGFP-1
pGFP-C1
pGFP-C2
pGFP-C3
pGFP-N1
pGFP-N2
pGFP-N3
phGFP-s65T
pIRES-EGFP
pNeoEGFP
pRSGFP-C1
pS65T-C1 (d)
p8op-lacZ
pACT2
pAS2 (d)
pAS2-1(d)
pB42 AD
pBridge
pCMV-HA
pCMV-Myc
pEZM3
pGAD GH
pGAD GL AD
pGAD10
pGAD424
pGADT7
pGADT7-Rec
pGBKT7
pGBT9
pGilda
pHISi
pHISi-1
pLacZi
pLexA
pVP16
pLAPSN
pLEGFP-C1
pLEGFP-N1
pLHCX
pUB
pLNCX
pLNCX2
pLNHX
pLPCX
pLXIN
pLXRN
pLXSN
pMSCVhyg
pMSCVneo
pMSCVpuro
pSIR
pVSV-G
pBI
pBI-EGFP
pBI-G
pBI-GL
pBI-L
pRetro-Off
pRetro-On
pRevTRE
pRev-tTA-IN
pRevTet-Off
pRevTet-Off-IN
pRevTet-On
pRevTRE2
pTet-Off
pTet-On
pTet-tTS
pTK-Hyg
pTRF
pTRE-HA
pTRE-6xHN
pTRE-d2EGFP
pTRE-Myc
pTRB2
pTRE2hyg
pTRE2hyg-Luc
pTRE2pur
pTRE2pur-Luc
pEXP1
pLIB
pT-Adv
pTriplEx
pTriplEx2
G. Amersham Pharmacia Biotech
pGEX-2T
pGEX-2TK
pGEX-3X
pGEX-1lambdaT
pGEX-4T-1
pGEX-4T-2
pGEX-4T-3
pGEX-5X-1
pGEX-5X-2
pGEX-5X-3
pGEX-6P-1
pGEX-6P-2
pGEX-6P-3

IV. Use

Adaptors according to the invention are used to ligate together two ends of a nucleic acid molecule or to ligate together two or more nucleic acid molecules.

A ligation reaction according to the invention is performed as follows. A mix comprising

4 μl of vector (10-200 ng)

3 μl of Insert (5-200 ng)

1 μl 10× Ligation Buffer

1 μl of Ligase (1-5 “Weiss” Units)

1 μl of Adaptor (1 ng)

10 μl of H2O

20 μl=Total is prepared and incubated at room temperature for 3-24 hrs or at 17° C. overnight. The formation of a ligation product is determined by transforming bacterial cells with an aliquot of the ligation reaction in a transformation reaction according to methods well known in the art (i.e., (described in Ausubel et al., 1995, Short Protocols in Molecular Biology, 3rd Edition, John Wiley & Sons) and Sambrook, Fritsch & Maniatis, 1989, Molecular Cloning: A Laboratory Manual, Second Edition) and as described in Example 3, below, and plating the transformed bacteria onto an LB plate containing the appropriate selection antibiotic. Nucleic acid is isolated from any resulting colonies and digested with the appropriate restriction enzyme(s) to determine the presence of an appropriately sized nucleic acid. The size of the digestion products are determined by electrophoresis on an agarose gel in the presence of ethidium bromide.

A similar protocol is used to ligate nucleic acid molecules comprising sticky ends or blunt ends.

Alternatively, low melt ligation is carried out as described in Example 3, below.

Two or more ready-to-use adaptors according to the invention can be provided in a kit format or an array format. The adaptors of a kit or an array can be grouped as in Tables 1-9. Such groups are designed to facilitate various types of cloning reactions. The adaptors of a kit or an array are individually compartmentalized. The adaptors of a kit are provided in combination with appropriate packaging means. Each adaptor of a kit of the invention is supplied at a concentration such that the adaptor can be added directly to the reaction of interest without being diluted or concentrated. The concentration of the adaptor in the kit supports, and is preferably optimal, for the reaction of interest to be performed with the adaptor. The concentration of an adaptor in a kit is from 100 ng/μl to 0.001 ng/μl, preferably from 10 ng/μl to 0.01 ng/μl, and most preferably from 5 ng/μl to 0.1 ng/μl. Each adaptor of a kit of the invention is supplied in an appropriate buffer such that the adaptor can be added directly to the reaction of interest in the adaptor storage buffer. An appropriate buffer includes any buffer that supports and is preferably optimal for the reaction of interest (for example a ligation reaction). Suitable adaptor storage buffers comprise a salt concentration of less than 1M, preferably less than 500 mM and most preferably less than 200 mM. The pH of a suitable adaptor storage buffer of the invention is between 7 and 8, preferably between 7.2 and 7.8 and most preferably between 7.4 and 7.6. A suitable adaptor storage buffer useful according to the invention includes but is not limited to medium restriction buffer (MRB) (defined herein).

A kit comprising two adaptors can be used to clone an insert wherein a different adaptor is ligated to each end of the insert. An additional use for a kit comprising two adaptors is for performing a two step ligation. A kit comprising two adaptors is also useful for immunoprecipitation experiments that require the cloning of a control and a test protein and transfection experiments that require the cloning of a test plasmid and a control plasmid (i.e., a wild-type and a mutant gene, or a test gene and a reporter gene).

The invention also provides for kits comprising more than two adaptors. Such kits are useful for analysis of the function of a gene promoter wherein a series of deletion mutants of the promoter is generated by individual cloning reactions, each of which requires ligation with a different adaptor. Kits comprising more than two adaptors are also useful for performing internal deletion mutagenesis analysis. Multiple part ligations (i.e., for cloning two or more nucleic acid molecules) can be carried out using a kit of the invention comprising more than two adaptors. A subclone library of DNA fragments for sequencing can be prepared using a kit of the invention comprising more than two adaptors. Kits comprising more than two adaptors, wherein each adaptor comprises a start or a stop signal located between the first and second end of the adaptor, are useful for producing deletion mutants (i.e., amino or carboxyl terminal, respectively) of a protein wherein each deleted form of the protein is generated in an individual cloning reaction and each cloning reaction comprises a ligation step with a different adaptor. Kits comprising two or more adaptors, wherein each adaptor comprises a start or a stop signal located between the first and second end of the adaptor are also useful for producing fusion proteins. Kits comprising two or more adaptors, wherein each adaptor maintains an open reading frame between the first and second end of the adaptor, are useful for producing internal deletion mutants of a protein wherein each deleted form of the protein is generated in an individual cloning reaction and each cloning reaction comprises a ligation step with a different adaptor.

### EXAMPLES

The invention is illustrated by the following nonlimiting examples wherein the following materials and methods are employed. The entire disclosure of each of the literature references cited hereinafter are incorporated by reference herein.

### Example 1

Oligonucleotide Synthesis

The following example describes the design and synthesis of an oligonucleotide pair useful according to the invention.

The members of an oligonucleotide pair, wherein each oligonucleotide has a first end, a second end and a central region, are designed according to the parameters described in the section entitled “Oligonucleotides”. For example, the members of an oligonucleotide pair are designed such that they anneal to form an adaptor comprising a first end and a second end, wherein the first end is compatible with a nucleic acid that has been digested with any of the restriction enzymes presented in the section entitled “Adaptors” and the second end is compatible with a nucleic acid that has been digested with any of the restriction enzymes presented in the section entitled “Adaptors”. An oligonucleotide according to the invention can form an adaptor comprising a first end and a second end, wherein at least one of the first and second ends are compatible with a nucleic acid comprising a sequence that is compatible with a nucleic acid digested with a restriction enzyme. An oligonucleotide can be designed to include a start codon (i.e., ATG) or a stop codon (TGA, TAG or TAA) between the first and second ends. In one embodiment, an oligonucleotide is designed to include nucleic acid sequence encoding a tag in the central region. In another embodiment, an oligonucleotide is designed to include a nucleic acid sequence that is derived from a gene of interest in the central region. Alternatively, an oligonucleotide is designed to include a nucleic acid sequence that encodes any amino acid sequence that maintains the proper reading frame.

Each oligonucleotide of the pair is between 6 to 100 nucleotides in length. The two oligonucleotides of an oligonucleotide pair are designed such that they anneal to form an adaptor comprising at least 2 and less than 90 contiguous hybridizing base pairs.

An oligonucleotide of the invention is prepared by any of the methods of synthesis described in the section entitled “Oligonucleotides”.

### Example 2

Adaptor Preparation

The following example describes how to prepare an adaptor according to the invention.

An adaptor is formed by annealing an oligonucleotide pair, prepared as described in Example 1. An oligonucleotide is annealed to form an adaptor comprising a first end and a second end, wherein the first and second end are compatible to a nucleic acid digested with a restriction enzyme or to a nucleic acid comprising a nucleic acid sequence that is compatible with a nucleic acid digested with a restriction enzyme.

Annealing is performed by mixing 1 μg/ml of one oligonucleotide and an equimolar amount of the second oligonucleotide. For example, a 12-mer oligonucleotide and a 16-mer oligonucleotide are annealed as follows:

A mix comprising

25 μl Oligo 1

33 μl Oligo 2 [=25 μl (Oligo 1)×16/12]

10 μl 10× MRB

32 μl H₂O

100 μl=Total (Final concentration is 0.58 μg/μl) is prepared in an eppendorf tube and placed in a rack in a 68° C. water bath for 10 minutes. After 10 minutes at 68° C., the adaptors are slow cooled to room temperature (ca. 22° C.). A portion of the stock of adaptor is kept frozen. A dilution comprising a working concentration of 1 ng/μl in 1× MRB (e.g. for above example, add 2 μl of adaptor to 50 μl 10× MRB+450 μl H₂O) is also prepared and stored at 4° C. or preferably −20° C. MRB is prepared as described in the section entitled “Adaptors”.

In certain embodiments, an adaptor according to the invention is phosphorylated as follows.

Phosphorylation is carried out by preparing the following mixture:

1 μl Adaptor (1-100 ng)

1.5 μl Polynucleotide Kinase Buffer (10×)

1 μl Polynucleotide Kinase

1 μl ATP (10 mM)

10.5 μl H₂O

15 μl Total

The mixture is incubated 1 hour at 37° C. A kinase buffer useful according to the invention comprises 70 mM Tris-HCl (pH 7.6), 10 mM MgCl₂, 5 mM dithiothreitol.

An adaptor is preferably stored in solution at a concentration of 1 ng/μl in 1× MRB at 4° C. or preferably −20° C. Alternatively, an adaptor can be stored in a lyophilized form wherein a solution of an adaptor (1-2 ng/μl in 1× MRB) is dried down (i.e., in an eppendorf tube) for example in a speed-vac, according to methods well known in the art. The lyophilized adaptor can be stored at room temperature, preferably at 4° C. and more preferably at −20° C.

An adaptor according to the invention can be individually compartmentalized for storage, for example in an eppendorf tube, in a well of a plate (i.e., a 96 well plate), in a glass jar or a capillary tube or on a piece of filter paper.

### Example 3

Ligation Reaction

The example that follows describes how to perform a ligation reaction according to the invention.

A ligation reaction useful for ligation of either blunt or sticky-ends, according to the invention is performed as described in the section entitled, “Adaptors”. Alternatively, a low-melt ligation reaction can be performed as follows.

I. Plasmid Restriction Digestion

| | Vector Plasmid | Insert Plasmid |
|---|---|---|
| DNA | 0.5 μl (0.5 μg) | 1-5 μl (1-5 μg) |
| 10XRB (usually 10X HRB) | 3 μl | 3 μl |
| Enzyme 1 | 1.5 μl | 1.5 μl |
| Enzyme 2 | 1.5 μl | — |
| Enzyme 3 | — | 1.5 μl |
| RNase | 3 μl (if mini prep DNA) | 3 μl (if mini prep |
| DNA) | | |
| H₂O | 20-23 μl | 16-23 μl |
| Total | | |

Preferably the ratio of fragments is approximately equimolar (although fairly significant differences will also work). Preferably 0.5 μg of vector is digested [this lower amount helps prevent overloading of the gel and therefore allows better separation of the fragment of interest from low amounts of single cut and supercoiled plasmid (which cause significant background)]. The amount of insert plasmid digested depends on the size of the insert and is primarily a consideration for detection issues. If the insert is between 150-800 bp, approximately 5 μg of plasmid is digested. If the insert is 800-2000 bp, approximately 2 μg is digested. If the plasmid is bigger than that, approximately 0.5-1 μg is digested.

II. Agarose Gel Electrophoresis

1) Preferably 1% Low Melting Point (LMP) Agarose is used unless the insert size is less than 300 bp (in which case, 1.5% LMP is used).

2) Gel electrophoresis is performed in TAE Buffer

3) Fragments are separated fairly well. Separation of the vector is most important in order to isolate a fragment from a minute amount of either single cut or supercoiled DNA which can cause significant background.

4) After running the gel, the gel piece containing the fragment of interest is isolated (using a clean razor blade) by minimizing the amount of surrounding agarose (minimizing the amount of excess agarose tends to increase the concentration of fragment and provides better purity) and each gel piece is placed into a separate eppendorf tube.

5) Minimize exposure of fragments to UV light.

III. Ligation Reaction

i. Eppendorf tubes containing gel pieces are placed into a 68° C. water bath for 5 minutes. If the temperature is slightly below 68° C., the gel pieces will not melt well.

ii. When gel pieces are melted, the tubes are removed one at a time, mixed briefly by flicking and the appropriate amount (see below) of the mixture is aliquoted to the CNTL and INSERT tubes. Gel pieces are stored for future experiments at −20° C.

iii. CNTL and INSERT tubes are incubated in the 68° C. water bath for 3-5 minutes.

iv. During this incubation period, Ligation Reaction Mix (see below) is prepared.

v. After CNTL and INSERT tubes are incubated at 68° C. for 3-5 minutes and the Ligation Reaction Mix has been prepared, the CNTL, INSERT, and Ligation Reaction Mix tubes are transferred to a 37° C. water bath and incubated at 37° C. for 5 minutes.

vi. While all of the tubes remain at 37° C., 10 μl of the ligation mix is pipetted and immediately transferred to the CNTL tube. The CNTL tube is mixed immediately by flicking and incubated at room temperature.

vii. 10 μl of ligation mix is transferred to the INSERT tube (as in vi.) and the INSERT tube is incubated at room temperature.

Two part ligations are incubated from 3-24 hours. Three or more part ligations are incubated for 12-24 hours.

For two part ligations the control tube contains 10 μl of the vector gel piece and the INSERT tube contains 5 μl of the vector gel piece and 5 μl of the insert gel piece. For a two part ligation with an adaptor the CNTL tube contains 5 μl of vector gel piece and 5 μl of insert gel piece and the INSERT tube contains 5 μl of the vector gel piece, 5 μl of the insert gel piece and 1 μl of the Adaptor.

The Adaptor should be added immediately after adding the ligation mix to the ligation fragments. The adaptor should not be added at the same time that the other fragments are added because the mixture is too warm and the adaptor will unanneal. If using two adaptors in a single ligation reaction, the adaptors must either be phosphorylated or 1 μl of polynucleotide kinase must be added to each ligation reaction.

For three part ligations the CNTL tube contains 5 μl of vector gel piece and 5 μl of one of the insert gel pieces and the INSERT tube contains 4 μl of the vector gel piece and 3 μl of each of the two insert gel pieces.

Ligation Reaction Mix:

| Per Reaction | 3X | 5X | 7X | 9X | |
|---|---|---|---|---|---|
| 6 μl H₂O | 18 μl | 30 μl | 42 μl | 54 μl | H₂O |
| 2 μl 10X Lig Buff (with New England Biolabs Ligase**) | 6 μl | 10 μl | 14 μl | 18 μl | 10X |
| Lig Buff | | | | | |
| 2 μl Ligase (New England Biolabs Cat# 202L, 400 μ/μl) | 6 μl | 10 μl | 14 μl | 18 μl | Ligase |
| 10 μl | | | | | |

The ligase buffer is warmed in a 37° C. water bath for a few minutes to get the DTT into solution.

IV. Transformation of Competent Bacteria

1) After ligation, the tubes are incubated at 68° C. for 5-10 minutes.

2) The tubes are removed individually and immediately mixed with 180 μl of Transformation Buffer (kept at 4° C.). The contents of the tube are pipetted up and down 4-5 times and the tube is put on ice.

3) After adding Transformation Buffer to each ligation, the tubes are incubated on ice for 15 minutes to cool to 4° C.

4) 100 μl of competent cells are added to each tube and mixed well by flicking (because of the diluted agarose, the solution is semi-solidified; but this can be disrupted by mixing well by flicking after the addition of competent cells).

5) Tubes are incubated on ice for 30 minutes.

6) Tubes are heat shocked at 37° C. for 2.5 minutes and then transferred back to an ice bucket.

7) The mixture of each tube is transferred to a sterile tube containing 1 ml of 2×YT, mixed briefly by flicking, and incubated in a 37° C. water bath for 45-60 minutes.

8) 300 μl of the mixture is plated onto an ampicillin containing 100 mm 2×YT plate.

9) The plate is incubated overnight at 37° C. (note: 300 ul of solution is generally too much to absorb well into agar so the plate is not inverted during overnight incubation

this allows the plate to dry slightly and allows the solution to soak in better).

10) The following day, colonies are picked and used to inoculate cultures for mini prep analysis.

Solutions

| 10X Low Restriction Buffer (10XLRB) | 10 ml of 10X LRB |
|---|---|
| 100 mM Tris-Cl (pH 7.5) | 1 ml 1 M Tris (pH 7.5) |
| 100 mM MgCl₂ | 1 ml 1 M MgCl₂ |
| 10 mM DTT | 100 ul 1 M DTT |
| 10X Medium Restriction Buffer (10XMRB) | 10 ml of 10X MRB |
| 100 mM Tris-Cl (pH 7.5) | 1 ml 1 M Tris (pH 7.5) |
| 500 mM NaCl | 1 ml 5 M NaCl |
| 100 mM MgCl | 1 ml 1 M MgCl₂ |
| 10 mM DTT | |
| 10X High Restriction Buffer (10XHRB) | 10 ml of 10X HRB |
| 500 mM Tris-Cl (pH 7.5) | 5 ml 1 M Tris (pH 7.5) |
| 1 M NaCl | 2 ml 5 M NaCl |
| 100 mM MgCl₂ | 1 ml 1 M MgCl₂ |
| 10 mM DTT | |

Ligase Buffer

The ligase buffer that is supplied by New England Biolabs Ligase (Cat# 202L, 400 μ/μl) is suitable for the methods of the invention.

Transformation Buffer

10 mM Hepes (pH 7.7)

50 mM CaCl₂

10% Glycerol

50× TAE

1 L

242 g Tris Base

57.1 ml Glacial Acetic Acid

100 ml 0.5M EDTA (pH 8.0)

| 2xYT | 1 L |
|---|---|
| tryptone | 16 g |
| yeast extract | 10 g |
| NaCl | 5 g |

### Example 4

Kit Preparation

The following example describes the preparation of a kit comprising two or more adaptors according to the invention.

A kit comprising two or more adaptors is prepared by synthesizing the appropriate adaptors as described in Example 2 above, individually compartmentalizing the adaptors and combining them with appropriate packaging means. The components of a kit of the invention are any combination of at least two adaptors. An adaptor that is supplied in a kit is in solution in an appropriate buffer, for example 1× MRB, preferably at a concentration of 1-2 ng/μl.

Useful kits of the invention comprise the following:

Standard restriction enzyme kit- each end of the adaptor is compatible with a nucleic acid digested with a standard restriction enzyme

Blunt kit- each end of the adaptor is compatible with a nucleic acid digested with a restriction enzyme that generates a blunt end

Frequent-Cutting enzyme kit- each end of the adaptor is compatible with a frequent-cutting restriction enzyme

Infrequent-Cutting enzyme kit- each end of the adaptor is compatible with an infrequent-cutting enzyme

5′ Overhang kit- each end of the adaptor is compatible with an enzyme that generates a 5′ overhang

3′ Overhang kit- each end of the adaptor is compatible with an enzyme that generates a 3′ overhang.

A kit wherein the first and second end of the adaptor are compatible with a nucleic acid digested with any restriction enzyme known in the art, including but not limited to any restriction enzyme disclosed in the section entitled, “Adaptors” can be prepared as described above.

A kit wherein the first end of the adaptor is compatible with a nucleic acid digested with a blunt-cutting enzyme, a standard enzyme, an infrequent cutting enzyme, a frequent cutting enzyme, an enzyme that generates a 5′ overhang, an enzyme that generates a 3′ overhang, or an enzyme that digests the polylinker of many commonly used cloning vectors, and the second end of the adaptor is compatible with a nucleic acid digested with a blunt-cutting restriction enzyme.

A kit wherein the first end of the adaptor is compatible with a nucleic acid digested with a blunt-cutting enzyme, a standard enzyme, an infrequent cutting enzyme, a frequent cutting enzyme, an enzyme that generates a 5′ overhang, an enzyme that generates a 3′ overhang, or an enzyme that digests the polylinker of many commonly used cloning vectors, and the second end of the adaptor is compatible with a nucleic acid digested with a standard restriction enzyme.

A kit wherein the first end of the adaptor is compatible with a nucleic acid digested with a blunt-cutting enzyme, a standard enzyme, an infrequent cutting enzyme, a frequent cutting enzyme, an enzyme that generates a 5′ overhang, an enzyme that generates a 3′ overhang, or an enzyme that digests the polylinker of many commonly used cloning vectors, and the second end of the adaptor is compatible with a nucleic acid digested with a frequent-cutting restriction enzyme.

A kit wherein the first end of the adaptor is compatible with a nucleic acid digested with a blunt-cutting enzyme, a standard enzyme, an infrequent cutting enzyme, a frequent cutting enzyme, an enzyme that generates a 5′ overhang, an enzyme that generates a 3′ overhang, or an enzyme that digests the polylinker of many commonly used cloning vectors, and the second end of the adaptor is compatible with a nucleic acid digested with an infrequent-cutting restriction enzyme.

A kit wherein the first end of the adaptor is compatible with a nucleic acid digested with a blunt-cutting enzyme, a standard enzyme, an infrequent cutting enzyme, a frequent cutting enzyme, an enzyme that generates a 5′ overhang, an enzyme that generates a 3′ overhang, or an enzyme that digests the polylinker of many commonly used cloning vectors, and the second end of the adaptor is compatible with a nucleic acid digested with a restriction enzyme that generates a 5′ overhang.

A kit wherein the first end of the adaptor is compatible with a nucleic acid digested with a blunt-cutting enzyme, a standard enzyme, an infrequent cutting enzyme, a frequent cutting enzyme, an enzyme that generates a 5′ overhang, an enzyme that generates a 3′ overhang, or an enzyme that digests the polylinker of many commonly used cloning vectors, and the second end of the adaptor is compatible with a nucleic acid digested with a restriction enzyme that generates a 3′ overhang.

A kit wherein the first end of the adaptor is compatible with a nucleic acid digested with a blunt-cutting enzyme, a standard enzyme, an infrequent cutting enzyme, a frequent cutting enzyme, an enzyme that generates a 5′ overhang, an enzyme that generates a 3′ overhang, or an enzyme that digests the polylinker of many commonly used cloning vectors, and the second end of the adaptor is compatible with a nucleic acid digested with a restriction enzyme that digests the polylinker of many commonly used cloning vectors.

A kit wherein the first and second ends of the adaptor are compatible with a nucleic acid comprising at least one end that is compatible with a nucleic acid digested with any restriction enzyme known in the art, including but not limited to any restriction enzyme disclosed in the section entitled, “Adaptors” can be prepared as described above.

Kits comprising adaptors wherein each adaptor comprises a start codon, a start codon, a tag or a nucleic acid sequence derived from a gene of interest can also be prepared as described above.

Kits comprising adaptors wherein each adaptor comprises a nucleic acid sequence encoding any amino acid sequence of interest that maintains a proper reading frame can also be prepared as described above.

Any of the kits described hereinabove can be prepared.

### Example 5

Cloning an Insert by Ligation of an Adaptor to Each End of the Insert

The following example describes how to use a kit comprising two adaptors to clone an insert by ligating a first adaptor to one end of the insert and a second adaptor to the opposite end of the insert.

To clone an insert into a vector, it is required that each end of the insert have compatibility with one respective end of the vector. In the event that neither end of the insert is compatible with either of the vector ends, two distinct adaptors can be used to link each end of the insert with one respective end of the vector. In this case, the vector and the insert are added to the ligation reaction, and two individual adaptors are added each of which serves to link one end of the insert to one respective end of the vector.

An insert can be cloned into a pUC19 vector (New England Biolabs) that has been digested with EcoR1 and BamH1. For example, an insert comprising a first end that is compatible with a nucleic acid sequence that has been digested with HindII and a second end that is compatible with a nucleic acid sequence that has been digested with XbaI is incubated with a first adaptor comprising the sequence 5′-GATCCGCCTGCAGCA-3′/3′-GCGGACGTCGTTCGA-5′, a second adaptor comprising the sequence 5′-AATTCGCCTGCAGCT-3-/3′-GCGGACGTCGAGATC-5′ and pUC19 that has been digested with EcoR1 and BamH1.

pUC19 is digested with EcoR1 and BamH1 as described in example 3. Ligation is carried out as described in Example 3. The appropriate adaptors are selected and an aliquot of each adaptor (provided in a ready-to-use format) is added to the ligation reaction mixture in an amount described in Example 3. Preferably, for multiple part ligations wherein more than one adaptor is used, the adaptors are phosphorylated as described in the section entitled “Adaptors” prior to performing the ligation reaction.

A portion of the ligation mixture is transformed as described in Example 3 and nucleic acid isolated from the transformants is analyzed by restriction enzyme digestion with EcoR1 and BamH1 and agarose gel electrophoresis. If the digestion generates two fragments, one that is equivalent in size to the original insert and one that is equivalent in size to the EcoRI/BamH1 digested pUC19, this indicates that the ligation reaction has worked.

### Example 6

Performing a Two-Step Ligation

The following example describes how to use a kit comprising two adaptors for a two-part ligation reaction.

Commonly, the ultimate desired cloning configuration can only be achieved through more than one cloning step (i.e. through more than one set of ligation, transformation, and screening procedures). For the first cloning step, an adaptor can be used to make the first insert compatible with the vector of interest. After screening and identifying the first appropriate cloning product, this vector (which is now ligated to the first insert) is then digested with the appropriate enzymes and a second insert is made compatible with this vector using a second cloning adaptor.

This example describes the introduction of two different pieces of DNA [the Rous Sarcoma Virus promoter and the human p/caf gene fragment] into the vector, pBluescript (Stratagene) to form an expression cassette driving the expression of the EBV BZLF1 gene.

The Rous Sarcoma Virus (RSV) Promoter fragment is excised from the plasmid, pUC-RSV-CAT by digesting with the enzymes BspEI and Hind III. The vector, pBluescript KS(+) (Stratagene) is digested with HincII and HindIII. The adaptor comprising the sequence, 5′-CCGGACCTGCAGCGC-3′/3′-TGGACGTCGCGCCGG-5′ (referred to as the “Blunt/BspEI Adaptor”) is used to link the blunt end generated by digesting pUC19-RSV-CAT with HincII to the end of pBluescript KS(+) that is digested with BspEI.

In the next cloning step, the resulting recombinant plasmid is digested with HindIII and NotI and the plasmid, pcDNA3-GAL4-p/caf, is digested with HindIII and ApaI to excise the p/caf gene. The p/caf gene is ligated to the HindIII and NotI digested vector using an ApaI/NotI adaptor (5′-CCCTGCAGCGC-3′/3′-CCGGGGGACGTCGCGCCGG-5′).

pBluescript KS(+) is digested with BspEI and HindIII and the RSV promoter is excised from pUC19-RSV-CAT by digesting with HincII and HindIII. The ligation is carried out as described in Example 3. The “Blunt/BspEI Adaptor” (provided in a ready-to-use-format) is added to the ligation reaction in an appropriate amount described in Example 3.

A portion of the ligation mixture is transformed as described in Example 3 and nucleic acid isolated from the transformants is analyzed by restriction enzyme digestion with BspEI and HindIII and agarose electrophoresis. If the digestion generates two fragments, one that is equivalent in size to the original insert (the RSV promoter) and one that is equivalent in size to the BspEI and HindIII digested pBluescript KS(+), this indicates that the ligation reaction has worked.

The DNA from this positive clone is used for the second step of the cloning experiment. This recombinant is digested with HindIII and NotI and pcDNA3-GAL4-p/caf is digested with HindIII and ApaI to excise the p/caf sequence. Ligation is carried out as described in Example 3. The ApaI/NotI adaptor (provided in ready-to-use format) is added to the ligation reaction mixture in an amount described in Example 3.

A portion of the ligation mixture is transformed as described in Example 3 and nucleic acid isolated from the transformants is analyzed by restriction enzyme digestion with HindIII and NotI followed by agarose gel electrophoresis. If the digestion generates two fragments, one that is equivalent in size to the original p/caf insert and one that is equivalent in size to the HindIII/NotI cut vector, this indicates that the ligation reaction has worked.

### Example 7

Cloning Two Genes or Two Proteins for a Transfection Assay or an Immunoprecipitation Assay

The following example describes how to use a kit comprising two adaptors to clone two genes or two proteins required for a transfection assay or an immunoprecipitation assay, respectively.

Two expression vectors, one comprising a wild-type gene and a second vector comprising a mutant gene are often required for assessment of in vivo gene function.

To address the function of a gene in a cell, the gene is cloned into an expression vector so that the corresponding protein can be generated in the cell after introducing the vector into the cell(s) of interest. To appropriately assess the function of the gene of interest, a second vector must be generated which does not express the gene of interest or does not express an essential domain of the gene of interest. Such a vector may lack an insert, or may contain a portion of the gene (i.e. a mutant) or may contain the gene cloned in the opposite orientation. Two adaptors can be used for this type of experiment, one for cloning the gene of interest into the vector and the other for generating the control vector.

For example, the Epstein Barr virus (EBV) BZLF1 gene which contains an NaeI (a blunt cutter) restriction endonuclease site upstream from the methionine initiation codon and contains a BamHI site downstream from its “stop” codon, is cloned in the sense direction in the expression vector, pcDNA3 (Invitrogen). The BZLF1 gene is excised from the plasmid, pBR322-BamZ, following digestion with NaeI and BamHI, pcDNA3 is digested with EcoRV (a blunt cutter) and NotI. The vector and insert are ligated in a reaction containing the adaptor, BamHI/NotI (5′ GATCCCCTGCAGCGC 3′/3′ GGGACGTCGCGCCGG 5′). A control vector for the pcDNA3-BZLF1 expression vector is generated by cloning only the amino terminal 24 amino acid encoding sequences of BZLF1 into pcDNA3. BZLF1 sequences encoding the first 24 amino acids of the reading frame is excised from pBR322-BamZ using NaeI and HindIII, pcDNA3 is digested with EcoRV and NotI and the BZLF1 amino terminal encoding sequences are ligated into pcDNA3 using the HindIII/NotI adaptor (5′ AGCTTCCTGCAGCGC 3′/3′ AGGACGTCGCGCCGG 5′) or the HindIII/STOP/NotI adaptor (5′ AGCTTGTGATTAGCTGAGGC 3′/3′ ACACTAATCGACTCCGCCGG 5′).

An immunoprecipitation experiment may require the preparation of a first expression construct that expresses a control protein and a second expression construct that expresses a test protein.

To identify proteins that interact with a protein of interest, the corresponding gene can be cloned into an expression vector, the expression vector can be introduced into cells, the cells can be metabolically labeled with a radioactive probe, the cells can be lysed and the protein of interest can be immunoprecipitated using an antibody that specifically recognizes the protein of interest. Following immunoprecipitation, the protein of interest plus any proteins that are bound to the protein of interest can be separated by SDS-PAGE (on a polyacrylamide gel). This approach is used for analyzing protein-protein interactions as well as for identifying novel proteins that interact with the protein of interest. This approach requires that the gene of interest be cloned into an expression vector. In addition, a control vector must also be generated which contains either no insert, a mutant gene of interest, or the gene of interest cloned in the reverse orientation. This experimental approach therefore requires that two separate vectors be generated—an expression vector expressing the wild type gene of interest and a control vector. An adaptor can be used for generating both the expression vector and the control vector, according to the ligation method presented in Example 3.

For example, the EBV BZLF1 gene which contains an NaeI (a blunt cutter) restriction endonuclease site upstream from its methionine initiation codon and a BamHI site down stream from its stop codon and is cloned in the sense direction in the expression vector, pcDNA3 (InVitrogen) by digesting pBR322-BamZ with NaeI and BamHI and isolating the 1234 bp NaeI/BamHI BZLF1 fragment, digesting pcDNA3 with EcoRV (a blunt cutter) and NotI, and ligating the vector and insert in a ligation reaction containing the adaptor, BamHI/NotI (5′ GATCCCCTGCAGCGC 3′/3′ GGGACGTCGCGCCGG 5′). A control vector for the pcDNA3-BZLF1 expression vector lacking the BZLF1 dimerization domain (which is essential for certain protein binding functions) is generated by excising the NaeI-PstI digestion fragment of BZLF1, and ligating with EcoRV and NotI digested pcDNA3 plus a NotI/PstI adaptor (5′ GGCCGCCGGATCCCCTGCA 3′/3′ CGGCCTAGGGG 5′) or a PstI/STOP/NotI adaptor (5′ GGTGATTAGCTGAGGC 3′/3′ ACGTCCACTAATCGACTCCGCCGG 5′).

### Example 8

Analysis of a Promoter Sequence

The following example describes how to use a kit comprising more than two adaptors to analyze a promoter sequence.

The mapping of functional promoter elements is typically carried out by generating a series of deletion mutants of the promoter of interest, introducing each mutant into cells separately and assessing the resulting promoter activity by S1 analysis or by analyzing reporter gene activity (e.g. chloramphenicol acetyltransferase activity, luciferase activity, beta-galactosidase activity). These studies require that the different promoter fragments be cloned in the same configuration in the same reporter plasmid vector. This process is facilitated by the use of an adaptor series, each member of which contains a common restriction site overhang on one end which is compatible with a useful site in the reporter vector, and a unique overhang on the other end which is compatible with one of a series of restriction site overhangs. Internal restriction sites are identified in the promoter of interest and ready-to-use adaptors are chosen which contain compatibility at their unique end with the internal site within the promoter. The appropriate adaptor is then used to clone each deletion mutant in the series into the common site within the reporter plasmid, according to the method described in Example 3.

For example, the human cyclin D2 promoter contains a NcoI site downstream from its transcriptional initiation site, a PstI site 891 bp upstream from the NcoI site, an XhoI site 985 bp upstream from the NcoI site, an ApaI site 1018 bp upstream from the NcoI site and a Sac1 site 1619 bp upstream from the NcoI site. Using a wild type promoter construct (pGL3Basic-cyclinD2p) containing the 1619 bp SacI/NcoI cyclin D2 promoter cloned into the SacI and BglII sites of pGL3Basic (the NcoI and BglII sites are blunted prior to ligation, thereby destroying both restriction sites), deletion mutants are generated as follows: The 3′ 985 bp of the cyclin D2 promoter is excised from pGL3Basic-cyclinD2p after digesting with XhoI and HindIII (which is immediately downstream from the destroyed BglII site in pGL3basic-cyclinD2p), the reporter vector, pGL3Basic (from Promega—without any insert), is digested with SmaI (a blunt cutter) and HindIII and the 985 bp cyclin D2 fragment is ligated into pGL3Basic using the XhoI/EcoRV adaptor (EcoRV end is blunt) (5′ TCGAGCCTGCAGCGAT 3′/3′ CGGACGTCGCTA 5′). The 3′ 891 bp of the cyclin D2 promoter is excised from pGL3Basic-cyclinD2p after digesting with PstI and HindIII, the reporter vector, pGL3Basic, is digested with SmaI and HindIII and the 891 bp cyclin D2 promoter fragment is ligated into pGL3Basic using the PstI/EcoRV adaptor (5′ TAACCTGCAGCGAT 3′/3′ TAATTGGACGTCGCTA 5′). The 3′ 518 bp of the cyclin D2 promoter is excised from pGL3Basic-cyclinD2p after digesting with ApaI and HindIII, the reporter vector, pGL3Basic, is digested with SmaI and HindIII and the 518 bp cyclin D2 fragment is ligated into pGL3Basic using the ApaI/EcoRV adaptor (5′ CCTGCAGCGAT 3′/3′ CCGGGGGACGTCGCTA 5′).

### Example 9

Generation of a Subclone Library of DNA Fragments for Sequencing

The following example describes how to use a kit comprising more than two adaptors to generate a library of DNA fragments for sequencing.

The generation of an overlapping set of fragment subclones derived from a DNA region of interest for the purposes of sequence analysis can be carried out using multiple cloning experiments whereby various fragment sub-regions are excised using different sets of restriction enzymes and these fragments are cloned into a vector for sequence analysis. Depending on the restriction enzyme recognition sequences present in the DNA region to be sequenced, distinct adaptors can be used to facilitate the cloning of each of these such sub-fragment, according to the method described in Example 3.

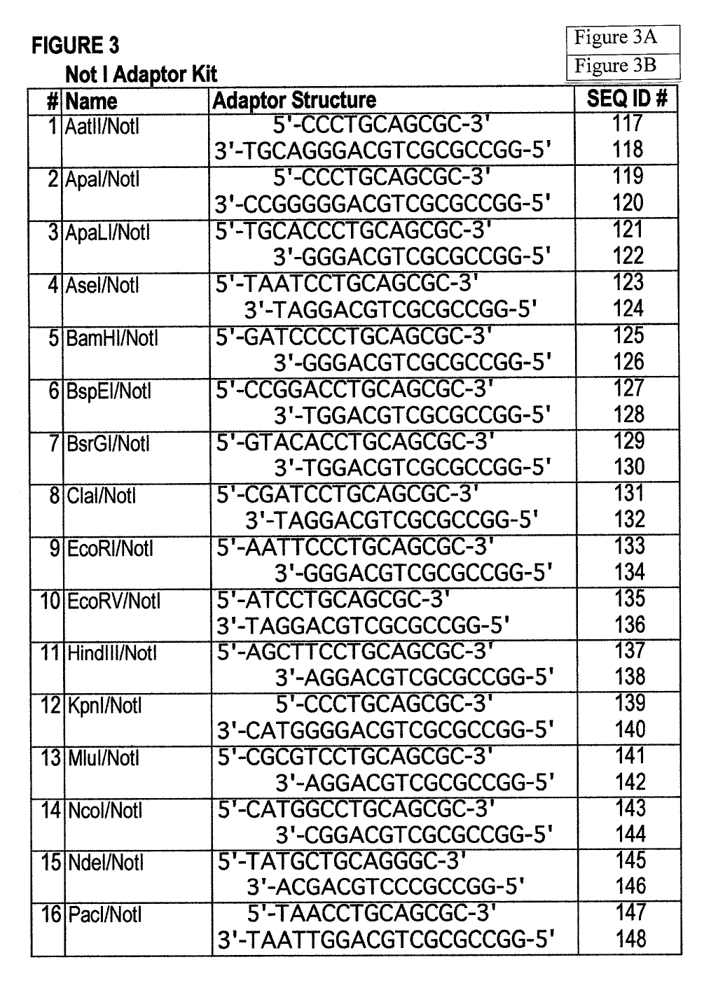

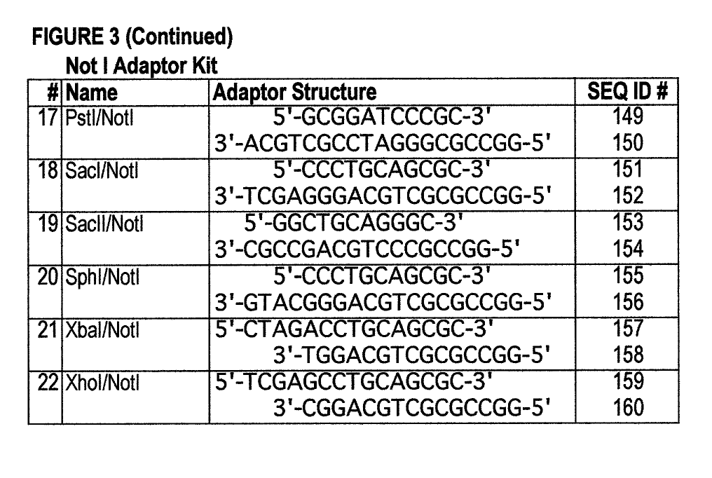

For example, if restriction mapping has shown that a 1 kb fragment X has a NotI at its 5′ end, a BspEI site 300 bp from the 5′ end, a NcoI site 600 bp from the 5′ end, an SphI site 800 bp from its 5′ end and an EcoRV site at its 3′ end, then subclones containing the 700 bp BspEI/EcoRV fragment, the 400 bp NcoI/EcoRV fragment, and the 200 bp SphI/EcoRV fragment can be cloned into NotI and EcoRV digested pcDNA3 using the adaptors, BspEI/NotI (5′ CCGGACCTGCAGCGC 3′/3′ TGGACGTCGCGCCGG 5′), NcoI/NotI (5′ CATGGCCTGCAGCGC 3′/3′ CGGACGTCGCGCCGG 5′), and SphlI/NotI (5′ CCCTGCAGCGC 3′/3′ GTACGGGACGTCGCGCCGG 5′), respectively (see FIG. 3). Successive sequence can be obtained using the T7 primer to obtain sequence the 5′ end of each cloned sub-fragment.

### Example 10

Production of Protein Deletions

The following example describes how to use a kit comprising at least three adaptors, wherein each adaptor provides a different reading frame for in frame cloning, and may further comprises an initiation sequence or a stop codon, to produce a panel of protein deletion mutants.

1. Amino terminal deletion mutagenesis.

Kits containing a series of ready-to-use adaptors with a common restriction site overhang on one end, a protein initiation sequence in the middle, and a unique restriction site overhang on the other end are useful for generating a series of gene deletions encoding amino terminal truncated proteins. One end of each adaptor must be compatible with a single site within the vector. Adaptors are chosen for this study such that the overhang on the opposite end is compatible with a restriction site found within the gene of interest. The deletion series is generated by excising different gene subfragments using different restriction enzymes and ligating the subfragment to the vector using the appropriate initiation adaptor, as described in Example 3.

Example: The plasmid pRC-HA-E2F1 contains a BamHI restriction endonuclease site immediately upstream from the initiation codon in the reading frame GGA TCC and a NotI site downstream from the stop codon of E2F1. The E2F1 gene contains a SacII site 246 bp downstream from the BamHI site in pRC-HA-E2F1 which is in the reading frame, CCG CGG, and a ClaI site which is 886 bp downstream from the BamHI site in pRC-HA-E2F1 which is in the reading frame, ATC GAT. To clone the entire reading frame of E2F1 and two amino terminal deletion mutants of E2F1 into the expression vector, pcDNA3, pRC-HA-E2F1 can be digested with BamHI and NotI, SacII and NotI, and ClaI and NotI, respectively. The corresponding E2F1 encoding sequences can be ligated to EcoRV and NotI digested pcDNA3 using the EcoRV/MetInit/BamHI adaptor, 5′-ATCGTCATGGCAG-3′/3′-TAGCAGTACCGTCCTAG-5′, the EcoRV/MetInit/SacII adaptor, 5′-ATCGTCATGGCACCGC-3′/3′-TAGCAGTACCGTGG-5′, and the EcoRV/MetInit/ClaI adaptor, 5′-ATCGTCATGGCAAT-3′/3′-TAGCAGTACCGTTAGC-5′), respectively.

2. Carboxyl terminal deletion mutagenesis.

Carboxyl terminal deletion mutants are generated as described above in the “amino terminal deletion mutagenesis” section except that the adaptors in the kit contain stop codons instead of initiation codons.

The plasmid, pRC-HA-E2F1 contains a HindIII site upstream from the initiation codon and a NotI site downstream from the E2F1 stop codon. The E2F1 gene contains a SacII restriction endonuclease site 240 bp downstream from its initiation codon, a HincII (a blunt cutter) restriction endonuclease site 452 bp downstream from its initiation codon, and a ClaI restriction endonuclease site 880 bp downstream from its initiation codon. To generate a carboxyl terminal deletion series for E2F1, the E2F1 fragments are isolated from pRC-HA-E2F1 after digesting with HindIII and SacII, HindIII and HincII, and HindIII and ClaI and these fragments are ligated to HindIII and NotI digested pcDNA3 using a SacII/STOP/NotI adaptor (5′ GGGTGATTAGCTGAGGC 3′/3′ CGCCCACTAATCGACTCCGCCGG 5′), a EcoRV/STOP/NotI adaptor (5′ ATCGTGATTAGCTGAGGC 3′/3′ TAGCACTAATCGACTCCGCCGG 5′), and a ClaI/STOP/NotI adaptor (5′ CGATGTGATTAGCTGAGGC 3′/3′ TACACTAATCGACTCCGCCGG 5′), respectively.

3. Internal deletion mutagenesis.

For internal deletion mutagenesis, adaptor kits comprising adaptors with overhangs for various restriction site overhangs on both ends are used. Internal deletions within a gene will be generated using adaptors that recognize each end of two internally cut gene fragments. Since the frame must be maintained, an adaptor with the appropriate spacing between the restriction site overhangs must be used.

The plasmid, pRC-HA-E2F1 contains a HindIII site upstream from the initiation codon and a NotI site downstream from the E2F1 stop codon. To generate internal deletion mutants of E2F1, the 5′ HindIII/BssHII E2F1 encoding fragment, the 5′ HindIII/SmaI E2F1 encoding fragment, and the 5′ HincII E2F1 encoding fragment are excised from pRC-HA-E2F1 and ligated to the BglII/NotI 3′ E2F1 encoding fragment (excised from pRC-HA-E2F1) and HindIII and NotI digested pcDNA3 using the BamHI/Mlul-Frame 3 (5′ GATCCGAGGCTGCAGGA 3′/3′ G CTCCGACGTCCTGCGC 5′), the BamHI/Stul(Blunt)-Frame 2 (5′ GATCCGGCTGCAGAGG 3′/3′ GCCGACGTCTCC 5′), and the BamHI/Stul(Blunt)-Frame 1 (5′ GATCCGCTGCAGAGG 3′/3′ GCGACGTCTCC 5′) adaptors, respectively (note that BglII digested DNA is compatible with a BamHI digested end, BssHII digested DNA is compatible with an MluI digested end, and that SmaI, HincI, and StuI give blunt ends).

Kits comprising at least three adaptors wherein each adaptor provides a different reading frame and further comprises an initiation sequence or a stop codon, are used, as described above, to generate “in frame fusion” proteins.

### Example 11

Multiple Part Ligation

The following example describes how to use a kit comprising more than two adaptors for a multiple part ligation reaction.

To clone two or more inserts, three or more adaptors may be used to facilitate compatibility. For two inserts, an adaptor can be used to join one end of insert A with the vector. A second adaptor can be used to join insert A with insert B, and a third adaptor can be used to join insert B with the vector.

Preferably, for multiple part ligations wherein more than one adaptor is used, the adaptors are phosphorylated as described in the section entitled “Adaptors” prior to performing the ligation reaction.

Two fragments are cloned into BamHI and EcoRI digested pUC19 in one ligation step using three different adaptors. One insert comprising a first end that is compatible with the nucleic acid sequence that has been digested with NotI and a second end that is compatible with a nucleic acid sequence that has been digested with AatII and a second insert comprising a first end that is compatible with the nucleic acid sequence EcoRV (Blunt) and a second end that is compatible with a nucleic acid sequence that has been digested with ApaI is combined with the BamHI/EcoRI digested pUC19 plus the following three adaptors., BamHI/NotI (5′-GATCCCCTGCAGCGC-3′/3′-GGGACGTCGCGCCGG-5′), AatII/EcoRV(Blunt) (5′-CCCTGCAGCGAT-3′/3′-TGCAGGGACGTCGCTA-5′),and ApaI/EcoRI (5′-CCCTGCAGCGG-3′/3′-CCGGGGGACGTCGCCTTAA-5′).

pUC19 is digested with EcoRI and BamHI as described in Example 3. Ligation is carried out as described in Example 3. The appropriate adaptors are selected and an aliquot of each adaptor (provided in a ready-to-use format) is added to the ligation reaction mixture in an amount described in Example 3. Preferably, for multiple part ligations wherein more than one adaptor is used, the adaptors are phosphorylated as described in the section entitled “Adaptors” prior to performing the ligation reaction.

A portion of the ligation mixture is transformed as described in Example 3 and nucleic acid isolated from the transformants is analyzed by restriction enzyme digestion with EcoRI and BamHI followed by agarose gel electrophoresis. If the digestion generates two fragments, one that is equivalent in size to the sum of the two inserts and one that is equivalent in size to the EcoRI/BamHI digested pUC19, this indicates that the ligation reaction has worked.

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims.

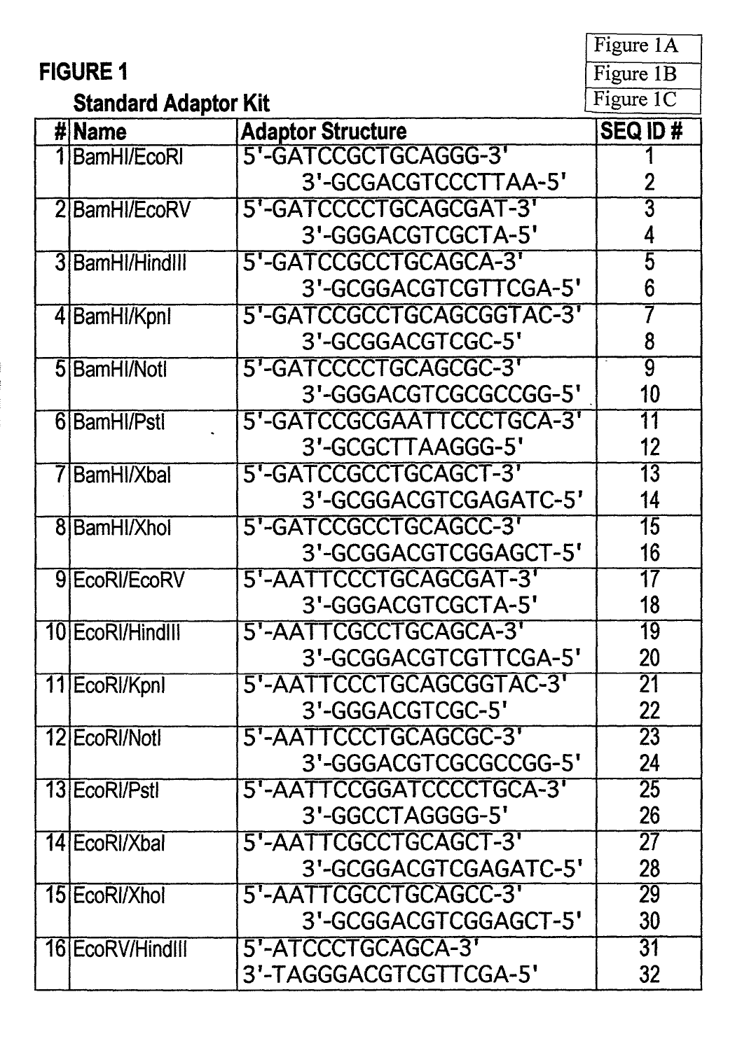

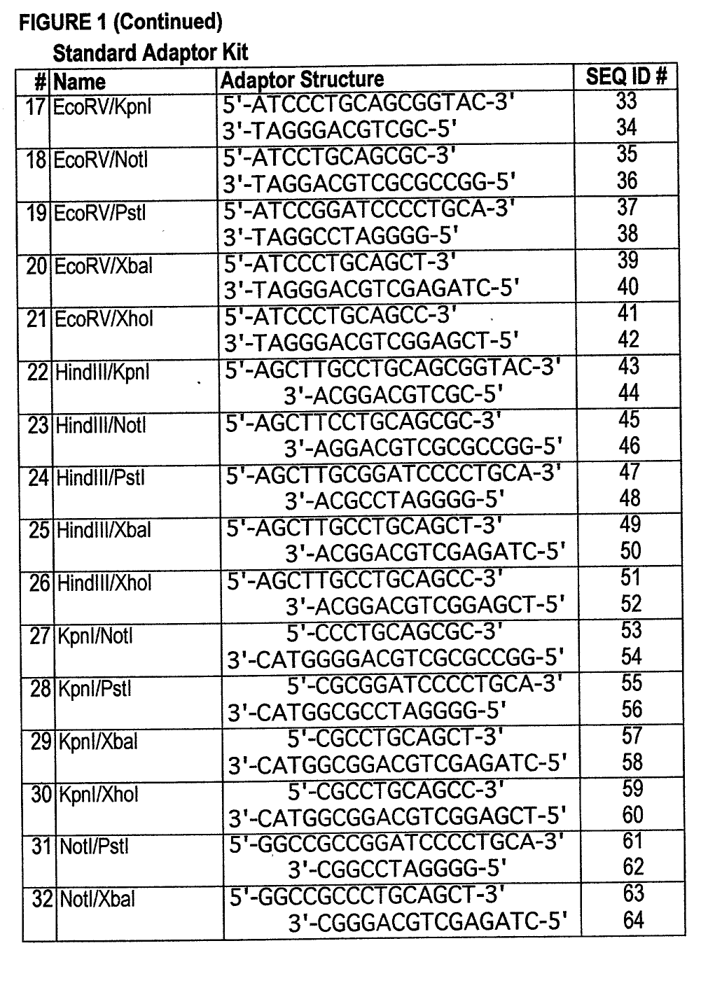

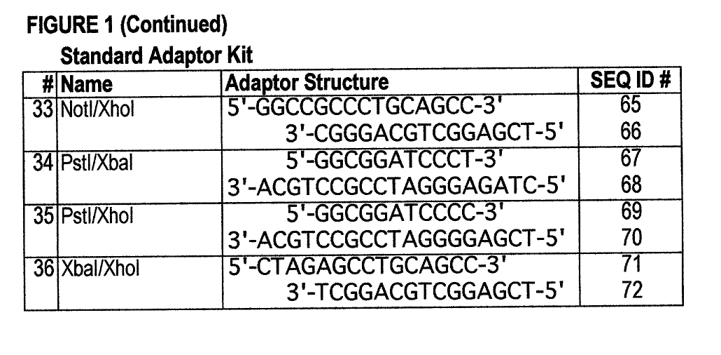

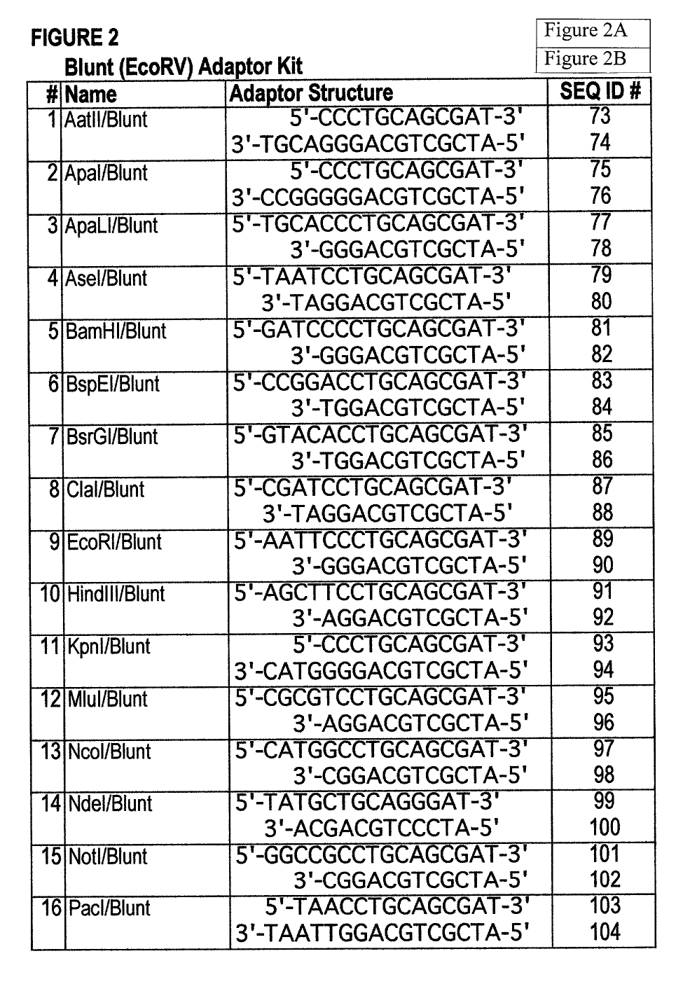

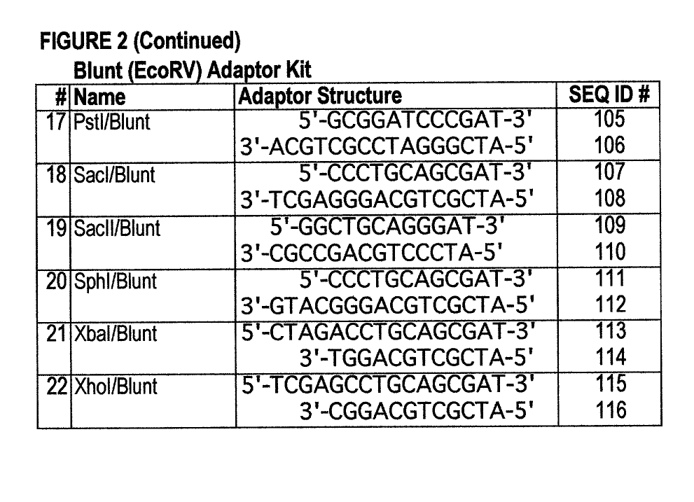

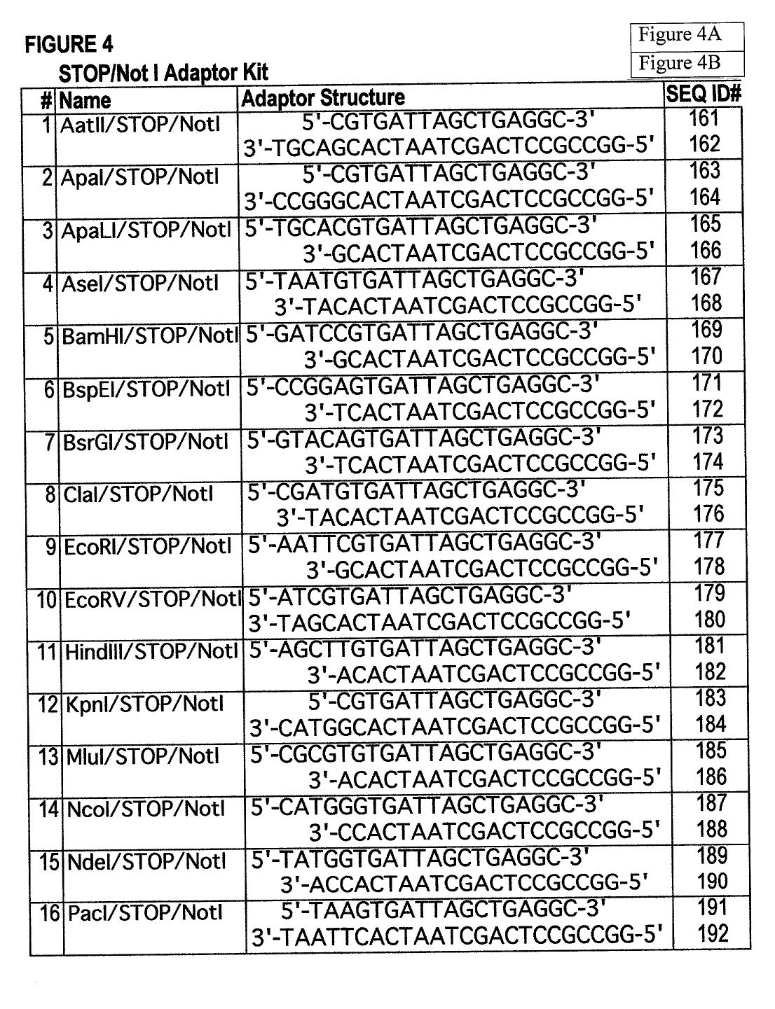

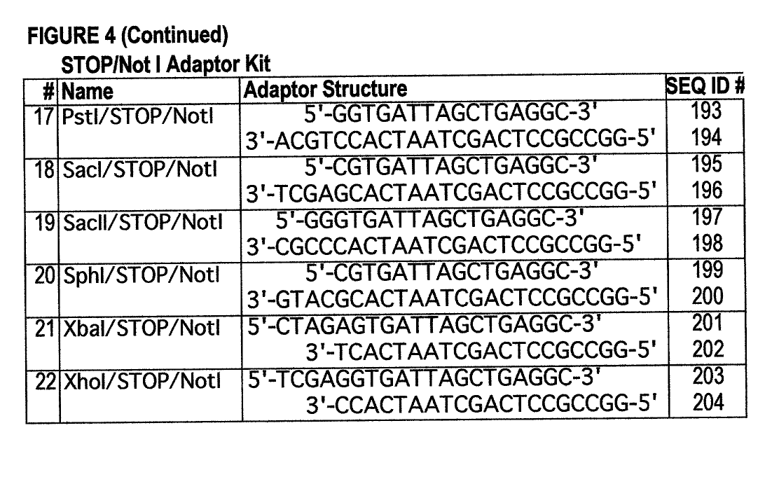

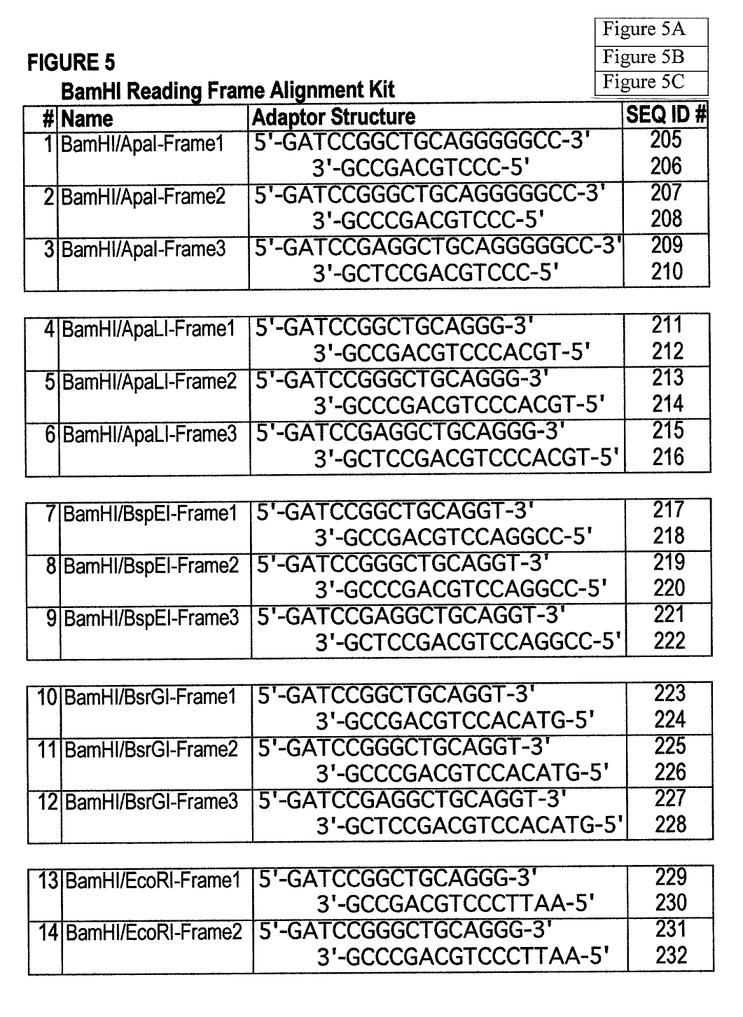

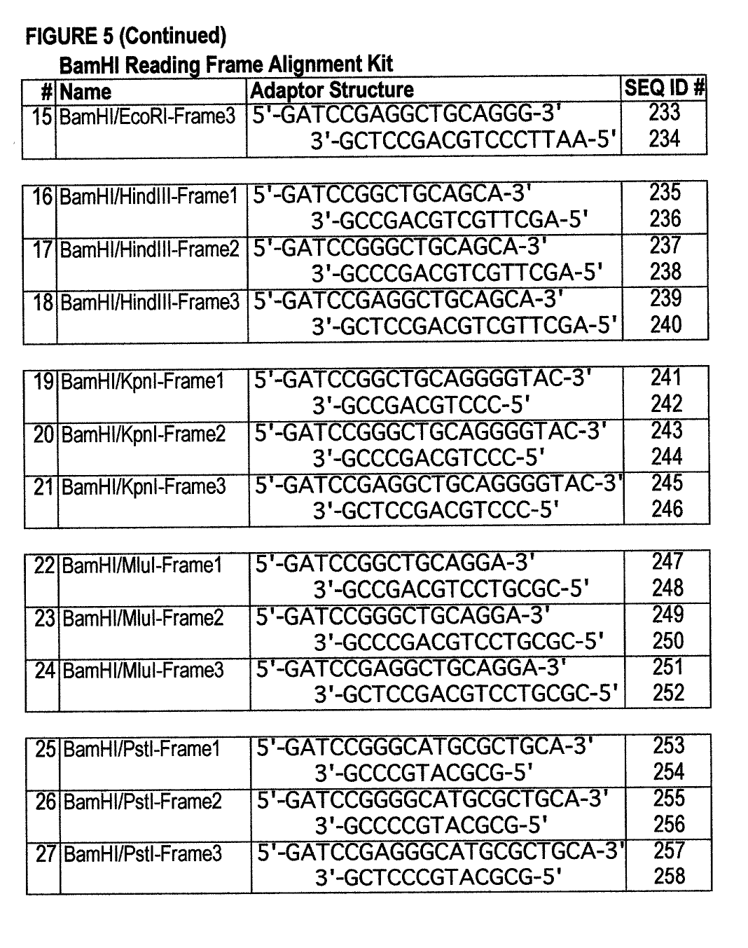

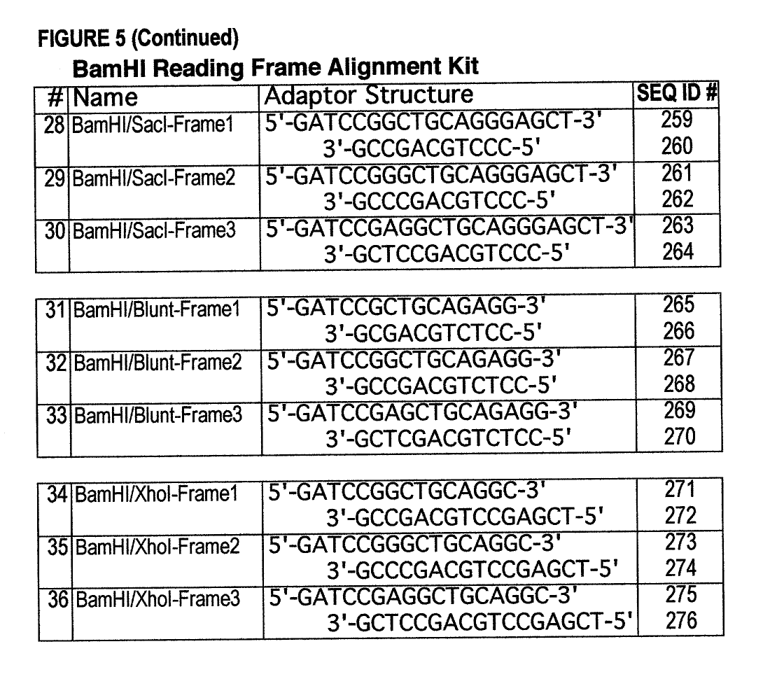

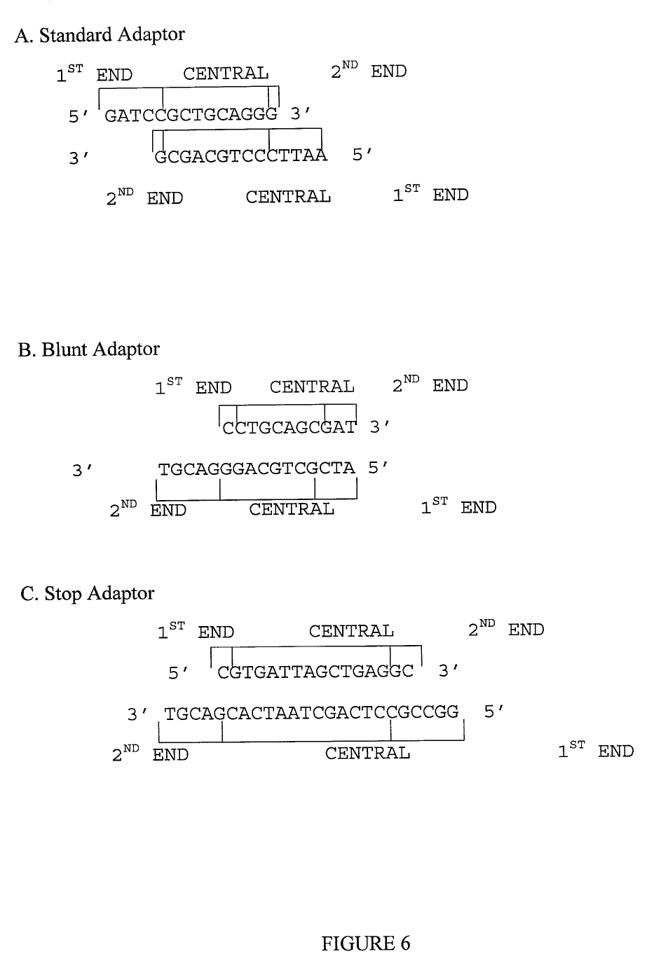

1. A composition comprising a pair of oligonucleotides wherein the first oligonucleotide of said pair comprises in 5′ to 3′ order a first end, a central region, and a second end, and the second oligonucleotide of said pair comprises in 5′ to 3′ order a first end, a central region, and a second end, wherein said central region of said first oligonucleotide of said pair is complementary to at least a portion of said central region of said second oligonucleotide of said pair; and
said first end of said first oligonucleotide and said second end of said second oligonucleotide of said pair individually comprises a nucleotide sequence that is either the sense or antisense strand of at least a portion of a double stranded recognition sequence that is compatible with a sample nucleic acid digested with a first restriction enzyme, and
said second end of said first oligonucleotide of said pair and said first end of said second oligonucleotide of said pair individually comprises a nucleotide sequence that is either the sense or antisense strand of at least a portion of a double stranded recognition sequence that is compatible with a sample nucleic acid digested with a second restriction enzyme,
wherein each of said pair of oligonucleotides is selected from the pairs of oligonucleotides presented in FIGS. 1-5.

2. A composition comprising at least two adaptors, wherein each adaptor comprises a first protruding nucleotide sequence at one end and a second protruding nucleotide sequence at the opposite end, and each of said first and second protruding nucleotide sequence are individually compatible with a sample nucleic acid digested with a restriction enzyme, and each adaptor is individually compartmentalized.

3. A composition comprising at least two adaptors, wherein each adaptor comprises a protruding nucleotide sequence that is compatible with a first sample nucleic acid digested with a restriction enzyme at one end and a second end comprising a nucleotide sequence that is compatible with a sample nucleic acid digested with a blunt-cutting restriction enzyme at the opposite end.

4. The composition of claim 2 wherein each adaptor comprises an identical first protruding nucleotide sequence that is compatible with a sample nucleic acid digested with a restriction enzyme wherein said restriction enzyme is selected from the group consisting of a standard restriction enzyme, an infrequent-cutting enzyme, a frequent-cutting enzyme, an enzyme that generates a 5′ overhang or an enzyme that generates a 3′ overhang.

5. The composition of claim 3 wherein each adaptor comprises an identical nucleotide sequence that is compatible with a sample nucleic acid digested with a blunt-cutting restriction enzyme.

6. The composition of claim 2 wherein said first and second protruding nucleotide sequence of each adaptor are individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

7. The composition of claim 3 wherein said protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

8. A composition comprising at least two adaptors wherein each adaptor comprises a first end and a second end, wherein each of said first and second end is individually compatible with a sample nucleic acid digested with a restriction enzyme, and wherein each of said at least two adaptors is selected from the adaptors presented in FIGS. 1-5.

9. The composition of claim 2, 3 or 8, wherein each of said at least two adaptors is phosphorylated.

10. An adaptor comprising a pair of annealed oligonucleotides having at one end a first protruding nucleotide sequence and at the opposite end a second protruding nucleotide sequence, wherein said first and second protruding nucleotide sequences are individually compatible with a sample nucleic acid digested with a recognition site for a restriction enzyme, and
further comprising at least one stop signal or at least one start signal located between said first and second protruding nucleotide sequence.

11. An adaptor comprising a pair of annealed oligonucleotides having at one end a protruding nucleotide sequence that is compatible with a sample nucleic acid digested with a restriction enzyme and at the opposite end a nucleotide sequence that is compatible with a nucleic acid sequence digested with a blunt-cutting restriction enzyme, and
further comprising at least one stop signal or at least one start signal located between said first and second protruding nucleotide sequences.

12. An adaptor comprising a pair of annealed oligonucleotides wherein each oligonucleotide of said pair has a first and a second end, wherein each of said first and second end is individually compatible with a sample nucleic acid digested with a restriction enzyme, and wherein said adaptor is selected from the adaptors presented in FIGS. 1-5.

13. The adaptor of claim 10, 11 or 12, wherein said adaptor is phosphorylated.

14. A kit comprising at least two pairs of oligonucleotides wherein each pair of said two pairs is individually compartmentalized,
wherein the first oligonucleotide of said pair comprises in 5′ to 3′ order a first end, a central region, and a second end, and the second oligonucleotide of said pair comprises in 5′ to 3′ order a first end, a central region, and a second end, wherein said central region of said first oligonucleotide of said pair is complementary to at least a portion of said central region of said second oligonucleotide of said pair; and
said first end of said first oligonucleotide and said second end of said second oligonucleotide of said pair individually comprises a nucleotide sequence that is either the sense or antisense strand of at least a portion of a double stranded recognition sequence that is compatible with a sample nucleic acid digested with a first restriction enzyme, and
said second end of said first oligonucleotide of said pair and said first end of said second oligonucleotide of said pair individually comprises a nucleotide sequence that is either the sense or antisense strand of at least a portion of a double stranded recognition sequence that is compatible with a sample nucleic acid digested with a second restriction enzyme, and
further comprising packaging means thereof.

15. The kit of claim 14 wherein each pair of oligonucleotides is annealed to form an adaptor wherein said adaptor comprises a first protruding nucleotide sequence at one end and a second protruding nucleotide sequence at the opposite end, and each of said first and second protruding nucleotide sequences is compatible with a sample nucleic acid digested with a restriction enzyme.

16. The kit of claim 14 wherein each adaptor comprises an identical first protruding nucleotide sequence that is compatible with a sample nucleic acid digested with a restriction enzyme wherein said restriction enzyme is selected from the group consisting of a standard restriction enzyme, a frequent-cutting restriction enzyme or an infrequent-cutting restriction enzyme.

17. The kit of claim 14 wherein said first and second protruding nucleotide sequence of each adaptor are individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

18. The kit of claim 14, wherein one of said restriction enzymes is a blunt-cutting restriction enzyme.

19. The kit of claim 18 wherein each pair of oligonucleotides is annealed to form an adaptor, wherein said adaptor comprises a protruding nucleotide sequence compatible with a sample nucleic acid digested with a restriction enzyme at one end and a nucleotide sequence compatible with a sample nucleic acid digested with a blunt-cutting restriction enzyme at the opposite end.

20. The kit of claim 19 wherein each adaptor comprises an identical nucleotide sequence that is compatible with a sample nucleic acid digested with a blunt-cutting restriction enzyme.

21. The kit of claim 19 wherein said protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

22. The kit of claim 14, wherein each of said at least two pairs of oligonucleotides is selected from the pairs of oligonucleotides presented in FIGS. 1-5.

23. A kit comprising at least two adaptors, wherein each of said two adaptors comprises a pair of annealed oligonucleotides and further comprises a first protruding nucleotide sequence at one end and a second protruding nucleotide sequence at the opposite end, and each of said first and second protruding nucleotide sequence is individually compatible with a sample nucleic acid digested with a restriction enzyme, and packaging means thereof.

24. A kit comprising at least two adaptors, wherein each of said two adaptors comprises a pair of annealed oligonucleotides and further comprises a protruding nucleotide sequence that is compatible with a sample nucleic acid digested with a restriction enzyme at one end and a sample nucleic acid digested with a blunt-cutting restriction enzyme at the opposite end, and packaging means thereof.

25. The kit of claim 23 wherein each adaptor comprises an identical first protruding nucleotide sequence that is compatible with a sample nucleic acid digested with a restriction enzyme, wherein said restriction enzyme is selected from the group consisting of a standard restriction enzyme, an infrequent-cutting enzyme, a frequent-cutting enzyme, an enzyme that generates a 5′ overhang or an enzyme that generates a 3′ overhang.

26. The kit of claim 24 wherein each adaptor comprises an identical nucleotide sequence that is compatible with a sample nucleic acid digested with a blunt-cutting restriction enzyme.

27. The kit of claim 23 wherein said first and second protruding nucleotide sequence of each adaptor is individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

28. The kit of claim 24 wherein said protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

29. A kit comprising at least two adaptors, wherein each of said at least two adaptors comprises a first end and a second end, and each of said first and second ends are individually compatible with a sample nucleic acid digested with a restriction enzyme, and wherein each of said at least two adaptors is selected from the adaptors presented in FIGS. 1-5.

30. The kit of claim 23, 24 or 29, wherein each of said at least two adaptors is phosphorylated.

31. An array comprising at least two pairs of oligonucleotides wherein each pair of said two pairs is individually compartmentalized,
wherein the first oligonucleotide of said pair comprises in 5′ to 3′ order a first end, a central region, and a second end, and the second oligonucleotide of said pair comprises in 5′ to 3′ order a first end, a central region, and a second end, wherein said central region of said first oligonucleotide of said pair is complementary to at least a portion of said central region of said second oligonucleotide of said pair; and
said first end of said first oligonucleotide and said second end of said second oligonucleotide of said pair individually comprises a nucleotide sequence that is either the sense or antisense strand of at least a portion of a double stranded recognition sequence that is compatible with a sample nucleic acid digested with a first restriction enzyme, and
said second end of said first oligonucleotide of said pair and said first end of said second oligonucleotide of said pair individually comprises a nucleotide sequence that is either the sense or antisense strand of at least a portion of a double stranded recognition sequence that is compatible with a sample nucleic acid digested with a second restriction enzyme.

32. The array of claim 31 wherein each pair of oligonucleotides is annealed to form an adaptor wherein said adaptor comprises a first protruding nucleotide sequence at one end and a second protruding nucleotide sequence at the opposite end, and each of said first and second protruding nucleotide sequences is compatible with a sample nucleic acid digested with a restriction enzyme.

33. The array of claim 31 wherein each adaptor comprises an identical first protruding nucleotide sequence that is compatible with a sample nucleic acid digested with a restriction enzyme wherein said restriction enzyme is selected from the group consisting of a standard restriction enzyme, a frequent-cutting restriction enzyme, an infrequent-cutting restriction enzyme, an enzyme that generates a 5′ overhang or an enzyme that generates a 3′ overhang.

34. The array of claim 31 wherein said first and second protruding nucleotide sequence of each adaptor are individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

35. The array of claim 31, wherein one of said restriction enzymes is a blunt-cutting restriction enzyme.

36. The array of claim 35 wherein each pair of oligonucleotides is annealed to form an adaptor, wherein said adaptor comprises a protruding nucleotide sequence compatible with a sample nucleic acid digested with a restriction enzyme at one end and a nucleotide sequence compatible with a sample nucleic acid digested with a blunt-cutting restriction enzyme at the opposite end.

37. The array of claim 35 wherein each adaptor comprises an identical nucleotide sequence that is compatible with a sample nucleic acid digested with a blunt-cutting restriction enzyme.

38. The array of claim 35 wherein said protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

39. The array of claim 31, wherein each of said at least two pairs of oligonucleotides is selected from the pairs of oligonucleotides presented in FIGS. 1-5.

40. A kit comprising at least two adaptors, wherein each of said two adaptors comprises a pair of annealed oligonucleotides and each of said two adaptors further comprises a first protruding nucleotide sequence at one end and a second protruding nucleotide sequence at the opposite end, and each of said first and second protruding nucleotide sequence is individually compatible with a sample nucleic acid digested with a restriction enzyme, and packaging means thereof.

41. A kit comprising at least two adaptors, wherein each of said two adaptors comprises a pair of annealed oligonucleotides and further comprises a protruding nucleotide sequence that is compatible with a sample nucleic acid digested with a restriction enzyme at one end and a sample nucleic acid digested with a blunt-cutting restriction enzyme at the opposite end, and packaging means thereof.

42. The kit of claim 40 wherein each adaptor comprises an identical first protruding nucleotide sequence that compatible with a sample nucleic acid digested with a restriction enzyme, wherein said restriction enzyme is selected from the group consisting of a standard restriction enzyme, an infrequent-cutting enzyme, a frequent-cutting enzyme, an enzyme that generates a 5′ overhang or an enzyme that generates a 3′ overhang.

43. The kit of claim 41 wherein each adaptor comprises an identical nucleotide sequence that is compatible with a sample nucleic acid digested with a blunt-cutting restriction enzyme.

44. The kit of claim 40 wherein said first and second protruding nucleotide sequence of each adaptor is individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

45. The kit of claim 41 wherein said protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

46. A kit comprising at least two adaptors, wherein each of said at least two adaptors comprises a first end and a second end, and each of said first and second ends are individually compatible with a sample nucleic acid digested with a restriction enzyme, and wherein each of said at least two adaptors is selected from the adaptors presented in FIGS. 1-5.

47. A kit comprising at least two adaptors, wherein each of said at least two adaptors comprises a first end and a second end, and each of said first and second ends are individually compatible with a sample nucleic acid digested with a restriction enzyme, wherein said restriction enzyme is selected from the group consisting of a standard restriction enzyme, an infrequent cutting enzyme, a frequent cutting enzyme, an enzyme that generates a 5′ overhang, an enzyme that generates a 3′ overhang or a blunt-cutting restriction enzyme, and
wherein said adaptor further comprises at least one start signal or at least one stop signal.

48. A kit comprising at least one set of three adaptors, wherein each adaptor of said set comprises an identical first end and an identical second end, and each of said first and second ends are individually compatible with a sample nucleic acid digested with a restriction enzyme, wherein said restriction enzyme is selected from the group consisting of a standard restriction enzyme, an infrequent cutting enzyme, a frequent cutting enzyme, an enzyme that generates a 5′ overhang, an enzyme that generates a 3′ overhang or a blunt-cutting restriction enzyme, and
wherein each of the first and second ends of each adaptor of said set are provided in a different reading frame.

49. The kit of claims 40, 41, 46, 47 and 48, wherein each of said at least two adaptors is phosphorylated.

50. A collection of at least two pairs of oligonucleotides wherein each pair of said two pairs is individually compartmentalized,
wherein the first oligonucleotide of said pair comprises in 5′ to 3′ order a first end, a central region, and a second end, and the second oligonucleotide of said pair comprises in 5′ to 3′ order a first end, a central region, and a second end, wherein said central region of said first oligonucleotide of said pair is complementary to at least a portion of said central region of said second oligonucleotide of said pair; and
said first end of said first oligonucleotide and said second end of said second oligonucleotide of said pair individually comprises a nucleotide sequence that is either the sense or antisense strand of at least a portion of a double stranded recognition sequence that is compatible with a sample nucleic acid digested with a first restriction enzyme, and
said second end of said first oligonucleotide of said pair and said first end of said second oligonucleotide of said pair individually comprises a nucleotide sequence that is either the sense or antisense strand of at least a portion of a double stranded recognition sequence that is compatible with a sample nucleic acid digested with a second restriction enzyme.

51. The collection of claim 50 wherein each pair of oligonucleotides is annealed to form an adaptor wherein said adaptor comprises a first protruding nucleotide sequence at one end and a second protruding nucleotide sequence at the opposite end, and each of said first and second protruding nucleotide sequences is compatible with a sample nucleic acid digested with a restriction enzyme.

52. The collection of claim 50 wherein each adaptor comprises an identical first protruding nucleotide sequence that is compatible with a sample nucleic acid digested with a restriction enzyme wherein said restriction enzyme is selected from the group consisting of a standard restriction enzyme, a frequent-cutting restriction enzyme, an infrequent-cutting restriction enzyme, an enzyme that generates a 5′ overhang or an enzyme that generates a 3′ overhang.

53. The collection of claim 50 wherein said first and second protruding nucleotide sequence of each adaptor are individually compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

54. The collection of claim 50, wherein one of said restriction enzymes is a blunt-cutting restriction enzyme.

55. The collection of claim 54 wherein each pair of oligonucleotides is annealed to form an adaptor, wherein said adaptor comprises a protruding nucleotide sequence compatible with a sample nucleic acid digested with a restriction enzyme at one end and a nucleotide sequence compatible with a sample nucleic acid digested with a blunt-cutting restriction enzyme at the opposite end.

56. The collection of claim 55 wherein each adaptor comprises an identical nucleotide sequence that is compatible with a sample nucleic acid digested with a blunt-cutting restriction enzyme.

57. The collection of claim 55 wherein said protruding nucleotide sequence is compatible with a sample nucleic acid digested with a restriction enzyme selected from the group consisting of a standard restriction enzyme, a frequent cutting enzyme, an infrequent-cutting enzyme, an enzyme that generates a 5′ overhang or an enzyme that generates a 3′ overhang.

58. The collection of claim 50, wherein each of said at least two pairs of oligonucleotides is selected from the pairs of oligonucleotides presented in FIGS. 1-5.

59. A method of using an adaptor comprising:
providing two nucleic acid molecules, each molecule having a 3′ end and a 5′ end wherein at least the 3′ or the 5′ end of said molecule is digested with a restriction enzyme, and an adaptor comprising two annealed oligonucleotides, wherein one end of said adaptor is compatible with one end of said first nucleic acid molecule, and the opposite end of said adaptor is compatible with one end of said second nucleic acid molecule,
incubating said nucleic acid molecules and said adaptor with a nucleic acid ligating activity under conditions wherein one end of said adaptor ligates to said first nucleic acid molecule and the opposite end of said adaptor ligates to said second nucleic acid molecule, and
detecting a ligation product.

60. A method of using the kit of claim 14 comprising:
providing two nucleic acid molecules, each molecule having a 3′ end and a 5′ end wherein at least the 3′ or the 5′ end of said molecule is digested with a restriction enzyme, and
selecting a pair of oligonucleotides from said kit and annealing said pair to form an adaptor, wherein one end of said adaptor is compatible with one end of said first nucleic acid molecule, and the opposite end of said adaptor is compatible with one end of said second nucleic acid molecule,
incubating said nucleic acid molecules and said adaptor with a nucleic acid ligating activity under conditions wherein one end of said adaptor ligates to said first nucleic acid molecule and the opposite end of said adaptor ligates to said second nucleic acid molecule, and
detecting a ligation product.

61. A method of using the array of claim 31 comprising:
providing two nucleic acid molecules, each molecule having a 3′ end and a 5′ end wherein at least the 3′ or the 5′ end of said molecule is digested with a restriction enzyme, and
selecting a pair of oligonucleotides from said array and annealing said pair to form an adaptor, wherein one end of said adaptor is compatible with one end of said first nucleic acid molecule, and the opposite end of said adaptor is compatible with one end of said second nucleic acid molecule,
incubating said nucleic acid molecules and said adaptor with a nucleic acid ligating activity under conditions wherein one end of said adaptor ligates to said first nucleic acid molecule and the opposite end of said adaptor ligates to said second nucleic acid molecule, and
detecting a ligation product.

62. A method of using the kit of claim 23 or 24 comprising:
providing two nucleic acid molecules, each molecule having a 3′ end and a 5′ end wherein at least the 3′ or the 5′ end of said molecule is digested with a restriction enzyme, and selecting an adaptor from said kit, wherein said adaptor comprises two annealed oligonucleotides, wherein one end of said adaptor is compatible with one end of said first nucleic acid molecule, and the opposite end of said adaptor is compatible with one end of said second nucleic acid molecule,
incubating said nucleic acid molecules and said adaptor with a nucleic acid ligating activity under conditions wherein one end of said adaptor ligates to said first nucleic acid molecule and the opposite end of said adaptor ligates to said second nucleic acid molecule, and
detecting a ligation product.

63. A method of preparing an adaptor comprising the steps of:
a) mixing a pair of oligonucleotides,
wherein the first oligonucleotide of said pair comprises in 5′ to 3′ order a first end, a central region, and a second end, and the second oligonucleotide of said pair comprises in 5′ to 3′ order a first end, a central region, and a second end, wherein said central region of said first oligonucleotide of said pair is complementary to at least a portion of said central region of said second oligonucleotide of said pair and said central region of said second oligonucleotide of said pair is complementary to at least a portion of said central region of said first oligonucleotide of said pair; and
said first end of each of said oligonucleotide of said pair individually comprises a nucleotide sequence that is either the sense or antisense strand of at least a portion of a double stranded recognition sequence that is compatible with a first sample nucleic acid digested with a first restriction enzyme, and
said second end of each of said oligonucleotide of said pair individually comprises a nucleotide sequence that is either the sense or antisense strand of at least a portion of a double stranded recognition sequence that is compatible with a second sample nucleic acid digested with a second restriction enzyme,
wherein one of said restriction enzymes is a blunt-cutting restriction enzyme; and
b) annealing said oligonucleotides of said pair to each other to form an adaptor.

64. The method of claim 63 wherein said adaptor comprises a first protruding nucleotide sequence compatible with a sample nucleic acid digested with a restriction enzyme at one end, and a nucleotide sequence compatible with a sample nucleic acid digested with a blunt-cutting restriction enzyme at the opposite end.

65. The method of claim 63 wherein the restriction enzyme that is not a blunt-cutting restriction enzyme is selected from the group consisting of a standard restriction enzyme, a frequent cutting enzyme, an infrequent-cutting enzyme, an enzyme that generates a 5′ overhang or an enzyme that generates a 3′ overhang.


SEQUENCE LISTING (276 sequences)

SEQ ID NO: 1 - DNA, 13 bp, Artificial Sequence
  Oligonucleotide
gatccgctgcagg

SEQ ID NO: 2 - DNA, 14 bp, Artificial Sequence
  Oligonucleotide
aattccctgcagcg

SEQ ID NO: 3 - DNA, 16 bp, Artificial Sequence
  Oligonucleotide
gatcccctgcagcgat

SEQ ID NO: 4 - DNA, 12 bp, Artificial Sequence
  Oligonucleotide
atcgctgcaggg

SEQ ID NO: 5 - DNA, 15 bp, Artificial Sequence
  Oligonucleotide
gatccgcctgcagca

SEQ ID NO: 6 - DNA, 15 bp, Artificial Sequence
  Oligonucleotide
agcttgctgcaggcg

SEQ ID NO: 7 - DNA, 19 bp, Artificial Sequence
  Oligonucleotide
gatccgcctgcagcggtac

SEQ ID NO: 8 - DNA, 11 bp, Artificial Sequence
  Oligonucleotide
cgctgcaggcg

SEQ ID NO: 9 - DNA, 15 bp, Artificial Sequence
  Oligonucleotide
gatcccctgcagcgc

SEQ ID NO: 10 - DNA, 15 bp, Artificial Sequence
  Oligonucleotide
ggccgcgctgcaggg

SEQ ID NO: 11 - DNA, 19 bp, Artificial Sequence
  Oligonucleotide
gatccgcgaattccctgca

SEQ ID NO: 12 - DNA, 11 bp, Artificial Sequence
  Oligonucleotide
gggaattcgcg

SEQ ID NO: 13 - DNA, 15 bp, Artificial Sequence
  Oligonucleotide
gatccgcctgcagct

SEQ ID NO: 14 - DNA, 15 bp, Artificial Sequence
  Oligonucleotide
ctagagctgcaggcg

SEQ ID NO: 15 - DNA, 15 bp, Artificial Sequence
  Oligonucleotide
gatccgcctgcagcc

SEQ ID NO: 16 - DNA, 15 bp, Artificial Sequence
  Oligonucleotide
tcgaggctgcaggcg

SEQ ID NO: 17 - DNA, 16 bp, Artificial Sequence
  Oligonucleotide
aattccctgcagcgat

SEQ ID NO: 18 - DNA, 12 bp, Artificial Sequence
  Oligonucleotide
atcgctgcaggg

SEQ ID NO: 19 - DNA, 15 bp, Artificial Sequence
  Oligonucleotide
aattcgcctgcagca

SEQ ID NO: 20 - DNA, 15 bp, Artificial Sequence
  Oligonucleotide
agcttgctgcaggcg

SEQ ID NO: 21 - DNA, 18 bp, Artificial Sequence
  Oligonucleotide
aattccctgcagcggtac

SEQ ID NO: 22 - DNA, 10 bp, Artificial Sequence
  Oligonucleotide
cgctgcaggg

SEQ ID NO: 23 - DNA, 16 bp, Artificial Sequence
  Oligonucleotide
aattcccctgcagcgc

SEQ ID NO: 24 - DNA, 15 bp, Artificial Sequence
  Oligonucleotide
ggccgcgctgcaggg

SEQ ID NO: 25 - DNA, 18 bp, Artificial Sequence
  Oligonucleotide
aattccggatcccctgca

SEQ ID NO: 26 - DNA, 10 bp, Artificial Sequence
  Oligonucleotide
ggggatccgg

SEQ ID NO: 27 - DNA, 15 bp, Artificial Sequence
  Oligonucleotide
aattcgcctgcagct

SEQ ID NO: 28 - DNA, 15 bp, Artificial Sequence
  Oligonucleotide
ctagagctgcaggcg

SEQ ID NO: 29 - DNA, 15 bp, Artificial Sequence
  Oligonucleotide
aattcgcctgcagcc

SEQ ID NO: 30 - DNA, 15 bp, Artificial Sequence
  Oligonucleotide
tcgaggctgcaggcg

SEQ ID NO: 31 - DNA, 12 bp, Artificial Sequence
  Oligonucleotide
atccctgcagca

SEQ ID NO: 32 - DNA, 16 bp, Artificial Sequence
  Oligonucleotide
agcttgctgcagggat

SEQ ID NO: 33 - DNA, 16 bp, Artificial Sequence
  Oligonucleotide
atccctgcagcggtac

SEQ ID NO: 34 - DNA, 12 bp, Artificial Sequence
  Oligonucleotide
cgctgcagggat

SEQ ID NO: 35 - DNA, 12 bp, Artificial Sequence
  Oligonucleotide
atcctgcagcgc

SEQ ID NO: 36 - DNA, 16 bp, Artificial Sequence
  Oligonucleotide
ggccgcgctgcaggat

SEQ ID NO: 37 - DNA, 16 bp, Artificial Sequence
  Oligonucleotide
atccggatcccctgca

SEQ ID NO: 38 - DNA, 12 bp, Artificial Sequence
  Oligonucleotide
ggggatccggat

SEQ ID NO: 39 - DNA, 12 bp, Artificial Sequence
  Oligonucleotide
atccctgcagct

SEQ ID NO: 40 - DNA, 16 bp, Artificial Sequence
  Oligonucleotide
ctagagctgcagggat

SEQ ID NO: 41 - DNA, 12 bp, Artificial Sequence
  Oligonucleotide
atccctgcagcc

SEQ ID NO: 42 - DNA, 16 bp, Artificial Sequence
  Oligonucleotide
tcgaggctgcagggat

SEQ ID NO: 43 - DNA, 19 bp, Artificial Sequence
  Oligonucleotide
agcttgcctgcagcggtac

SEQ ID NO: 44 - DNA, 11 bp, Artificial Sequence
  Oligonucleotide
cgctgcaggca

SEQ ID NO: 45 - DNA, 15 bp, Artificial Sequence
  Oligonucleotide
agcttcctgcagcgc

SEQ ID NO: 46 - DNA, 15 bp, Artificial Sequence
  Oligonucleotide
ggccgcgctgcagga

SEQ ID NO: 47 - DNA, 19 bp, Artificial Sequence
  Oligonucleotide
agcttgcggatcccctgca

SEQ ID NO: 48 - DNA, 11 bp, Artificial Sequence
  Oligonucleotide
ggggatccgca

SEQ ID NO: 49 - DNA, 15 bp, Artificial Sequence
  Oligonucleotide
agcttgcctgcagct

SEQ ID NO: 50 - DNA, 15 bp, Artificial Sequence
  Oligonucleotide
ctagagctgcaggca

SEQ ID NO: 51 - DNA, 15 bp, Artificial Sequence
  Oligonucleotide
agcttgcctgcagcc

SEQ ID NO: 52 - DNA, 15 bp, Artificial Sequence
  Oligonucleotide
tcgaggctgcaggca

SEQ ID NO: 53 - DNA, 11 bp, Artificial Sequence
  Oligonucleotide
ccctgcagcgc

SEQ ID NO: 54 - DNA, 19 bp, Artificial Sequence
  Oligonucleotide
ggccgcgctgcaggggtac

SEQ ID NO: 55 - DNA, 15 bp, Artificial Sequence
  Oligonucleotide
cgcggatcccctgca

SEQ ID NO: 56 - DNA, 15 bp, Artificial Sequence
  Oligonucleotide
ggggatccgcggtac

SEQ ID NO: 57 - DNA, 11 bp, Artificial Sequence
  Oligonucleotide
cgcctgcagct

SEQ ID NO: 58 - DNA, 19 bp, Artificial Sequence
  Oligonucleotide
ctagagctgcaggcggtac

SEQ ID NO: 59 - DNA, 11 bp, Artificial Sequence
  Oligonucleotide
cgcctgcagcc

SEQ ID NO: 60 - DNA, 19 bp, Artificial Sequence
  Oligonucleotide
tcgaggctgcaggcggtac

SEQ ID NO: 61 - DNA, 19 bp, Artificial Sequence
  Oligonucleotide
ggccgccggatcccctgca

SEQ ID NO: 62 - DNA, 11 bp, Artificial Sequence
  Oligonucleotide
ggggatccggc

SEQ ID NO: 63 - DNA, 15 bp, Artificial Sequence
  Oligonucleotide
ggccgccctgcagct

SEQ ID NO: 64 - DNA, 15 bp, Artificial Sequence
  Oligonucleotide
ctagagctgcagggc

SEQ ID NO: 65 - DNA, 15 bp, Artificial Sequence
  Oligonucleotide
ggccgccctgcagcc

SEQ ID NO: 66 - DNA, 15 bp, Artificial Sequence
  Oligonucleotide
tcgaggctgcagggc

SEQ ID NO: 67 - DNA, 11 bp, Artificial Sequence
  Oligonucleotide
ggcggatccct

SEQ ID NO: 68 - DNA, 19 bp, Artificial Sequence
  Oligonucleotide
ctagagggatccgcctgca

SEQ ID NO: 69 - DNA, 11 bp, Artificial Sequence
  Oligonucleotide
ggcggatcccc

SEQ ID NO: 70 - DNA, 19 bp, Artificial Sequence
  Oligonucleotide
tcgaggggatccgcctgca

SEQ ID NO: 71 - DNA, 15 bp, Artificial Sequence
  Oligonucleotide
ctagagcctgcagcc

SEQ ID NO: 72 - DNA, 15 bp, Artificial Sequence
  Oligonucleotide
tcgaggctgcaggct

SEQ ID NO: 73 - DNA, 12 bp, Artificial Sequence
  Oligonucleotide
ccctgcagcgat

SEQ ID NO: 74 - DNA, 16 bp, Artificial Sequence
  Oligonucleotide
atcgctgcagggacgt

SEQ ID NO: 75 - DNA, 12 bp, Artificial Sequence
  Oligonucleotide
ccctgcagcgat

SEQ ID NO: 76 - DNA, 16 bp, Artificial Sequence
  Oligonucleotide
atcgctgcagggggcc

SEQ ID NO: 77 - DNA, 16 bp, Artificial Sequence
  Oligonucleotide
tgcaccctgcagcgat

SEQ ID NO: 78 - DNA, 12 bp, Artificial Sequence
  Oligonucleotide
atcgctgcaggg

SEQ ID NO: 79 - DNA, 15 bp, Artificial Sequence
  Oligonucleotide
taatcctgcagcgat

SEQ ID NO: 80 - DNA, 13 bp, Artificial Sequence
  Oligonucleotide
atcgctgcaggat

SEQ ID NO: 81 - DNA, 16 bp, Artificial Sequence
  Oligonucleotide
gatcccctgcagcgat

SEQ ID NO: 82 - DNA, 12 bp, Artificial Sequence
  Oligonucleotide
atcgctgcaggg

SEQ ID NO: 83 - DNA, 16 bp, Artificial Sequence
  Oligonucleotide
ccggacctgcagcgat

SEQ ID NO: 84 - DNA, 12 bp, Artificial Sequence
  Oligonucleotide
atcgctgcaggt

SEQ ID NO: 85 - DNA, 16 bp, Artificial Sequence
  Oligonucleotide
gtacacctgcagcgat

SEQ ID NO: 86 - DNA, 12 bp, Artificial Sequence
  Oligonucleotide
atcgctgcaggt

SEQ ID NO: 87 - DNA, 15 bp, Artificial Sequence
  Oligonucleotide
cgatcctgcagcgat

SEQ ID NO: 88 - DNA, 13 bp, Artificial Sequence
  Oligonucleotide
atcgctgcaggat

SEQ ID NO: 89 - DNA, 16 bp, Artificial Sequence
  Oligonucleotide
aattccctgcagcgat

SEQ ID NO: 90 - DNA, 12 bp, Artificial Sequence
  Oligonucleotide
atcgctgcaggg

SEQ ID NO: 91 - DNA, 16 bp, Artificial Sequence
  Oligonucleotide
agcttcctgcagcgat

SEQ ID NO: 92 - DNA, 12 bp, Artificial Sequence
  Oligonucleotide
atcgctgcagga

SEQ ID NO: 93 - DNA, 12 bp, Artificial Sequence
  Oligonucleotide
ccctgcagcgat

SEQ ID NO: 94 - DNA, 16 bp, Artificial Sequence
  Oligonucleotide
atcgctgcaggggtac

SEQ ID NO: 95 - DNA, 16 bp, Artificial Sequence
  Oligonucleotide
cgcgtcctgcagcgat

SEQ ID NO: 96 - DNA, 12 bp, Artificial Sequence
  Oligonucleotide
atcgctgcagga

SEQ ID NO: 97 - DNA, 16 bp, Artificial Sequence
  Oligonucleotide
catggcctgcagcgat

SEQ ID NO: 98 - DNA, 12 bp, Artificial Sequence
  Oligonucleotide
atcgctgcaggc

SEQ ID NO: 99 - DNA, 14 bp, Artificial Sequence
  Oligonucleotide
tatgctgcagggat

SEQ ID NO: 100 - DNA, 12 bp, Artificial Sequence
  Oligonucleotide
atccctgcagca

SEQ ID NO: 101 - DNA, 16 bp, Artificial Sequence
  Oligonucleotide
ggccgcctgcagcgat

SEQ ID NO: 102 - DNA, 12 bp, Artificial Sequence
  Oligonucleotide
atcgctgcaggc

SEQ ID NO: 103 - DNA, 14 bp, Artificial Sequence
  Oligonucleotide
taacctgcagcgat

SEQ ID NO: 104 - DNA, 16 bp, Artificial Sequence
  Oligonucleotide
atcgctgcaggttaat

SEQ ID NO: 105 - DNA, 12 bp, Artificial Sequence
  Oligonucleotide
gcggatcccgat

SEQ ID NO: 106 - DNA, 16 bp, Artificial Sequence
  Oligonucleotide
atcgggatccgctgca

SEQ ID NO: 107 - DNA, 12 bp, Artificial Sequence
  Oligonucleotide
ccctgcagcgat

SEQ ID NO: 108 - DNA, 16 bp, Artificial Sequence
  Oligonucleotide
atcgctgcagggagct

SEQ ID NO: 109 - DNA, 12 bp, Artificial Sequence
  Oligonucleotide
ggctgcagggat

SEQ ID NO: 110 - DNA, 14 bp, Artificial Sequence
  Oligonucleotide
atccctgcagccgc

SEQ ID NO: 111 - DNA, 12 bp, Artificial Sequence
  Oligonucleotide
ccctgcagcgat

SEQ ID NO: 112 - DNA, 16 bp, Artificial Sequence
  Oligonucleotide
atcgctgcagggcatg

SEQ ID NO: 113 - DNA, 16 bp, Artificial Sequence
  Oligonucleotide
ctagacctgcagcgat

SEQ ID NO: 114 - DNA, 12 bp, Artificial Sequence
  Oligonucleotide
atcgctgcaggt

SEQ ID NO: 115 - DNA, 16 bp, Artificial Sequence
  Oligonucleotide
tcgagcctgcagcgat

SEQ ID NO: 116 - DNA, 12 bp, Artificial Sequence
  Oligonucleotide
atcgctgcaggc

SEQ ID NO: 117 - DNA, 11 bp, Artificial Sequence
  Oligonucleotide
ccctgcagcgc

SEQ ID NO: 118 - DNA, 19 bp, Artificial Sequence
  Oligonucleotide
ggccgcgctgcagggacgt

SEQ ID NO: 119 - DNA, 11 bp, Artificial Sequence
  Oligonucleotide
ccctgcagcgc

SEQ ID NO: 120 - DNA, 19 bp, Artificial Sequence
  Oligonucleotide
ggccgcgctgcagggggcc

SEQ ID NO: 121 - DNA, 15 bp, Artificial Sequence
  Oligonucleotide
tgcaccctgcagcgc

SEQ ID NO: 122 - DNA, 15 bp, Artificial Sequence
  Oligonucleotide
ggccgcgctgcaggg

SEQ ID NO: 123 - DNA, 14 bp, Artificial Sequence
  Oligonucleotide
taatcctgcagcgc

SEQ ID NO: 124 - DNA, 16 bp, Artificial Sequence
  Oligonucleotide
ggccgcgctgcaggat

SEQ ID NO: 125 - DNA, 15 bp, Artificial Sequence
  Oligonucleotide
gatcccctgcagcgc

SEQ ID NO: 126 - DNA, 15 bp, Artificial Sequence
  Oligonucleotide
ggccgcgctgcaggg

SEQ ID NO: 127 - DNA, 15 bp, Artificial Sequence
  Oligonucleotide
ccggacctgcagcgc

SEQ ID NO: 128 - DNA, 15 bp, Artificial Sequence
  Oligonucleotide
ggccgcgctgcaggt

SEQ ID NO: 129 - DNA, 15 bp, Artificial Sequence
  Oligonucleotide
gtacacctgcagcgc

SEQ ID NO: 130 - DNA, 15 bp, Artificial Sequence
  Oligonucleotide
ggccgcgctgcaggt

SEQ ID NO: 131 - DNA, 14 bp, Artificial Sequence
  Oligonucleotide
cgatcctgcagcgc

SEQ ID NO: 132 - DNA, 16 bp, Artificial Sequence
  Oligonucleotide
ggccgcgctgcaggat

SEQ ID NO: 133 - DNA, 15 bp, Artificial Sequence
  Oligonucleotide
aattccctgcagcgc

SEQ ID NO: 134 - DNA, 15 bp, Artificial Sequence
  Oligonucleotide
ggccgcgctgcaggg

SEQ ID NO: 135 - DNA, 12 bp, Artificial Sequence
  Oligonucleotide
atcctgcagcgc

SEQ ID NO: 136 - DNA, 16 bp, Artificial Sequence
  Oligonucleotide
ggccgcgctgcaggat

SEQ ID NO: 137 - DNA, 15 bp, Artificial Sequence
  Oligonucleotide
agcttcctgcagcgc

SEQ ID NO: 138 - DNA, 15 bp, Artificial Sequence
  Oligonucleotide
ggccgcgctgcagga

SEQ ID NO: 139 - DNA, 11 bp, Artificial Sequence
  Oligonucleotide
ccctgcagcgc

SEQ ID NO: 140 - DNA, 19 bp, Artificial Sequence
  Oligonucleotide
ggccgcgctgcaggggtac

SEQ ID NO: 141 - DNA, 15 bp, Artificial Sequence
  Oligonucleotide
cgcgtcctgcagcgc

SEQ ID NO: 142 - DNA, 15 bp, Artificial Sequence
  Oligonucleotide
ggccgcgctgcagga

SEQ ID NO: 143 - DNA, 15 bp, Artificial Sequence
  Oligonucleotide
catggcctgcagcgc

SEQ ID NO: 144 - DNA, 15 bp, Artificial Sequence
  Oligonucleotide
ggccgcgctgcaggc

SEQ ID NO: 145 - DNA, 13 bp, Artificial Sequence
  Oligonucleotide
tatgctgcagggc

SEQ ID NO: 146 - DNA, 15 bp, Artificial Sequence
  Oligonucleotide
ggccgccctgcagca

SEQ ID NO: 147 - DNA, 13 bp, Artificial Sequence
  Oligonucleotide
taacctgcagcgc

SEQ ID NO: 148 - DNA, 19 bp, Artificial Sequence
  Oligonucleotide
ggccgcgctgcaggttaat

SEQ ID NO: 149 - DNA, 11 bp, Artificial Sequence
  Oligonucleotide
gcggatcccgc

SEQ ID NO: 150 - DNA, 19 bp, Artificial Sequence
  Oligonucleotide
ggccgcgggatccgctgca

SEQ ID NO: 151 - DNA, 11 bp, Artificial Sequence
  Oligonucleotide
ccctgcagcgc

SEQ ID NO: 152 - DNA, 19 bp, Artificial Sequence
  Oligonucleotide
ggccgcgctgcagggagct

SEQ ID NO: 153 - DNA, 11 bp, Artificial Sequence
  Oligonucleotide
ggctgcagggc

SEQ ID NO: 154 - DNA, 17 bp, Artificial Sequence
  Oligonucleotide
ggccgccctgcagccgc

SEQ ID NO: 155 - DNA, 11 bp, Artificial Sequence
  Oligonucleotide
ccctgcagcgc

SEQ ID NO: 156 - DNA, 19 bp, Artificial Sequence
  Oligonucleotide
ggccgcgctgcagggcatg

SEQ ID NO: 157 - DNA, 15 bp, Artificial Sequence
  Oligonucleotide
ctagacctgcagcgc

SEQ ID NO: 158 - DNA, 15 bp, Artificial Sequence
  Oligonucleotide
ggccgcgctgcaggt

SEQ ID NO: 159 - DNA, 15 bp, Artificial Sequence
  Oligonucleotide
tcgagcctgcagcgc

SEQ ID NO: 160 - DNA, 15 bp, Artificial Sequence
  Oligonucleotide
ggccgcgctgcaggc

SEQ ID NO: 161 - DNA, 16 bp, Artificial Sequence
  Oligonucleotide
cgtgattagctgaggc

SEQ ID NO: 162 - DNA, 24 bp, Artificial Sequence
  Oligonucleotide
ggccgcctcagctaatcacgacgt

SEQ ID NO: 163 - DNA, 16 bp, Artificial Sequence
  Oligonucleotide
cgtgattagctgaggc

SEQ ID NO: 164 - DNA, 24 bp, Artificial Sequence
  Oligonucleotide
ggccgcctcagctaatcacgggcc

SEQ ID NO: 165 - DNA, 20 bp, Artificial Sequence
  Oligonucleotide
tgcacgtgattagctgaggc

SEQ ID NO: 166 - DNA, 20 bp, Artificial Sequence
  Oligonucleotide
ggccgcctcagctaatcacg

SEQ ID NO: 167 - DNA, 19 bp, Artificial Sequence
  Oligonucleotide
taatgtgattagctgaggc

SEQ ID NO: 168 - DNA, 21 bp, Artificial Sequence
  Oligonucleotide
ggccgcctcagctaatcacat

SEQ ID NO: 169 - DNA, 20 bp, Artificial Sequence
  Oligonucleotide
gatccgtgattagctgaggc

SEQ ID NO: 170 - DNA, 20 bp, Artificial Sequence
  Oligonucleotide
ggccgcctcagctaatcacg

SEQ ID NO: 171 - DNA, 20 bp, Artificial Sequence
  Oligonucleotide
ccggagtgattagctgaggc

SEQ ID NO: 172 - DNA, 20 bp, Artificial Sequence
  Oligonucleotide
ggccgcctcagctaatcact

SEQ ID NO: 173 - DNA, 20 bp, Artificial Sequence
  Oligonucleotide
gtacagtgattagctgaggc

SEQ ID NO: 174 - DNA, 20 bp, Artificial Sequence
  Oligonucleotide
ggccgcctcagctaatcact

SEQ ID NO: 175 - DNA, 19 bp, Artificial Sequence
  Oligonucleotide
cgatgtgattagctgaggc

SEQ ID NO: 176 - DNA, 21 bp, Artificial Sequence
  Oligonucleotide
ggccgcctcagctaatcacat

SEQ ID NO: 177 - DNA, 20 bp, Artificial Sequence
  Oligonucleotide
aattcgtgattagctgaggc

SEQ ID NO: 178 - DNA, 20 bp, Artificial Sequence
  Oligonucleotide
ggccgcctcagctaatcacg

SEQ ID NO: 179 - DNA, 18 bp, Artificial Sequence
  Oligonucleotide
atcgtgattagctgaggc

SEQ ID NO: 180 - DNA, 22 bp, Artificial Sequence
  Oligonucleotide
ggccgcctcagctaatcacgat

SEQ ID NO: 181 - DNA, 20 bp, Artificial Sequence
  Oligonucleotide
agcttgtgattagctgaggc

SEQ ID NO: 182 - DNA, 20 bp, Artificial Sequence
  Oligonucleotide
ggccgcctcagctaatcaca

SEQ ID NO: 183 - DNA, 16 bp, Artificial Sequence
  Oligonucleotide
cgtgattagctgaggc

SEQ ID NO: 184 - DNA, 24 bp, Artificial Sequence
  Oligonucleotide
ggccgcctcagctaatcacggtac

SEQ ID NO: 185 - DNA, 20 bp, Artificial Sequence
  Oligonucleotide
cgcgtgtgattagctgaggc

SEQ ID NO: 186 - DNA, 20 bp, Artificial Sequence
  Oligonucleotide
ggccgcctcagctaatcaca

SEQ ID NO: 187 - DNA, 20 bp, Artificial Sequence
  Oligonucleotide
catgggtgattagctgaggc

SEQ ID NO: 188 - DNA, 20 bp, Artificial Sequence
  Oligonucleotide
ggccgcctcagctaatcacc

SEQ ID NO: 189 - DNA, 19 bp, Artificial Sequence
  Oligonucleotide
tatggtgattagctgaggc

SEQ ID NO: 190 - DNA, 21 bp, Artificial Sequence
  Oligonucleotide
ggccgcctcagctaatcacca

SEQ ID NO: 191 - DNA, 18 bp, Artificial Sequence
  Oligonucleotide
taagtgattagctgaggc

SEQ ID NO: 192 - DNA, 24 bp, Artificial Sequence
  Oligonucleotide
ggccgcctcagctaatcacttaat

SEQ ID NO: 193 - DNA, 16 bp, Artificial Sequence
  Oligonucleotide
ggtgattagctgaggc

SEQ ID NO: 194 - DNA, 24 bp, Artificial Sequence
  Oligonucleotide
ggccgcctcagctaatcacctgca

SEQ ID NO: 195 - DNA, 16 bp, Artificial Sequence
  Oligonucleotide
cgtgattagctgaggc

SEQ ID NO: 196 - DNA, 24 bp, Artificial Sequence
  Oligonucleotide
ggccgcctcagctaatcacgagct

SEQ ID NO: 197 - DNA, 17 bp, Artificial Sequence
  Oligonucleotide
gggtgattagctgaggc

SEQ ID NO: 198 - DNA, 23 bp, Artificial Sequence
  Oligonucleotide
ggccgcctcagctaatcacccgc

SEQ ID NO: 199 - DNA, 16 bp, Artificial Sequence
  Oligonucleotide
cgtgattagctgaggc

SEQ ID NO: 200 - DNA, 24 bp, Artificial Sequence
  Oligonucleotide
ggccgcctcagctaatcacgcatg

SEQ ID NO: 201 - DNA, 20 bp, Artificial Sequence
  Oligonucleotide
ctagagtgattagctgaggc

SEQ ID NO: 202 - DNA, 20 bp, Artificial Sequence
  Oligonucleotide
ggccgcctcagctaatcact

SEQ ID NO: 203 - DNA, 20 bp, Artificial Sequence
  Oligonucleotide
tcgaggtgattagctgaggc

SEQ ID NO: 204 - DNA, 20 bp, Artificial Sequence
  Oligonucleotide
ggccgcctcagctaatcacc

SEQ ID NO: 205 - DNA, 19 bp, Artificial Sequence
  Oligonucleotide
gatccggctgcagggggcc

SEQ ID NO: 206 - DNA, 11 bp, Artificial Sequence
  Oligonucleotide
ccctgcagccg

SEQ ID NO: 207 - DNA, 20 bp, Artificial Sequence
  Oligonucleotide
gatccgggctgcagggggcc

SEQ ID NO: 208 - DNA, 12 bp, Artificial Sequence
  Oligonucleotide
ccctgcagcccg

SEQ ID NO: 209 - DNA, 21 bp, Artificial Sequence
  Oligonucleotide
gatccgaggctgcagggggcc

SEQ ID NO: 210 - DNA, 13 bp, Artificial Sequence
  Oligonucleotide
ccctgcagcctcg

SEQ ID NO: 211 - DNA, 15 bp, Artificial Sequence
  Oligonucleotide
gatccggctgcaggg

SEQ ID NO: 212 - DNA, 15 bp, Artificial Sequence
  Oligonucleotide
tgcaccctgcagccg

SEQ ID NO: 213 - DNA, 16 bp, Artificial Sequence
  Oligonucleotide
gatccgggctgcaggg

SEQ ID NO: 214 - DNA, 16 bp, Artificial Sequence
  Oligonucleotide
tgcaccctgcagcccg

SEQ ID NO: 215 - DNA, 17 bp, Artificial Sequence
  Oligonucleotide
gatccgaggctgcaggg

SEQ ID NO: 216 - DNA, 17 bp, Artificial Sequence
  Oligonucleotide
tgcaccctgcagcctcg

SEQ ID NO: 217 - DNA, 15 bp, Artificial Sequence
  Oligonucleotide
gatccggctgcaggt

SEQ ID NO: 218 - DNA, 15 bp, Artificial Sequence
  Oligonucleotide
ccggacctgcagccg

SEQ ID NO: 219 - DNA, 16 bp, Artificial Sequence
  Oligonucleotide
gatccgggctgcaggt

SEQ ID NO: 220 - DNA, 16 bp, Artificial Sequence
  Oligonucleotide
ccggacctgcagcccg

SEQ ID NO: 221 - DNA, 17 bp, Artificial Sequence
  Oligonucleotide
gatccgaggctgcaggt

SEQ ID NO: 222 - DNA, 17 bp, Artificial Sequence
  Oligonucleotide
ccggacctgcagcctcg

SEQ ID NO: 223 - DNA, 15 bp, Artificial Sequence
  Oligonucleotide
gatccggctgcaggt

SEQ ID NO: 224 - DNA, 15 bp, Artificial Sequence
  Oligonucleotide
gtacacctgcagccg

SEQ ID NO: 225 - DNA, 16 bp, Artificial Sequence
  Oligonucleotide
gatccgggctgcaggt

SEQ ID NO: 226 - DNA, 16 bp, Artificial Sequence
  Oligonucleotide
gtacacctgcagcccg

SEQ ID NO: 227 - DNA, 17 bp, Artificial Sequence
  Oligonucleotide
gatccgaggctgcaggt

SEQ ID NO: 228 - DNA, 17 bp, Artificial Sequence
  Oligonucleotide
gtacacctgcagcctcg

SEQ ID NO: 229 - DNA, 15 bp, Artificial Sequence
  Oligonucleotide
gatccggctgcaggg

SEQ ID NO: 230 - DNA, 15 bp, Artificial Sequence
  Oligonucleotide
aattccctgcagccg

SEQ ID NO: 231 - DNA, 16 bp, Artificial Sequence
  Oligonucleotide
gatccgggctgcaggg

SEQ ID NO: 232 - DNA, 16 bp, Artificial Sequence
  Oligonucleotide
aattccctgcagcccg

SEQ ID NO: 233 - DNA, 17 bp, Artificial Sequence
  Oligonucleotide
gatccgaggctgcaggg

SEQ ID NO: 234 - DNA, 17 bp, Artificial Sequence
  Oligonucleotide
aattccctgcagcctcg

SEQ ID NO: 235 - DNA, 15 bp, Artificial Sequence
  Oligonucleotide
gatccggctgcagca

SEQ ID NO: 236 - DNA, 15 bp, Artificial Sequence
  Oligonucleotide
agcttgctgcagccg

SEQ ID NO: 237 - DNA, 16 bp, Artificial Sequence
  Oligonucleotide
gatccgggctgcagca

SEQ ID NO: 238 - DNA, 16 bp, Artificial Sequence
  Oligonucleotide
agcttgctgcagcccg

SEQ ID NO: 239 - DNA, 17 bp, Artificial Sequence
  Oligonucleotide
gatccgaggctgcagca

SEQ ID NO: 240 - DNA, 17 bp, Artificial Sequence
  Oligonucleotide
agcttgctgcagcctcg

SEQ ID NO: 241 - DNA, 19 bp, Artificial Sequence
  Oligonucleotide
gatccggctgcaggggtac

SEQ ID NO: 242 - DNA, 11 bp, Artificial Sequence
  Oligonucleotide
ccctgcagccg

SEQ ID NO: 243 - DNA, 20 bp, Artificial Sequence
  Oligonucleotide
gatccgggctgcaggggtac

SEQ ID NO: 244 - DNA, 12 bp, Artificial Sequence
  Oligonucleotide
ccctgcagcccg

SEQ ID NO: 245 - DNA, 21 bp, Artificial Sequence
  Oligonucleotide
gatccgaggctgcaggggtac

SEQ ID NO: 246 - DNA, 13 bp, Artificial Sequence
  Oligonucleotide
ccctgcagcctcg

SEQ ID NO: 247 - DNA, 15 bp, Artificial Sequence
  Oligonucleotide
gatccggctgcagga

SEQ ID NO: 248 - DNA, 15 bp, Artificial Sequence
  Oligonucleotide
cgcgtcctgcagccg

SEQ ID NO: 249 - DNA, 16 bp, Artificial Sequence
  Oligonucleotide
gatccgggctgcagga

SEQ ID NO: 250 - DNA, 16 bp, Artificial Sequence
  Oligonucleotide
cgcgtcctgcagcccg

SEQ ID NO: 251 - DNA, 17 bp, Artificial Sequence
  Oligonucleotide
gatccgaggctgcagga

SEQ ID NO: 252 - DNA, 17 bp, Artificial Sequence
  Oligonucleotide
cgcgtcctgcagcctcg

SEQ ID NO: 253 - DNA, 19 bp, Artificial Sequence
  Oligonucleotide
gatccgggcatgcgctgca

SEQ ID NO: 254 - DNA, 11 bp, Artificial Sequence
  Oligonucleotide
gcgcatgcccg

SEQ ID NO: 255 - DNA, 20 bp, Artificial Sequence
  Oligonucleotide
gatccggggcatgcgctgca

SEQ ID NO: 256 - DNA, 12 bp, Artificial Sequence
  Oligonucleotide
gcgcatgccccg

SEQ ID NO: 257 - DNA, 21 bp, Artificial Sequence
  Oligonucleotide
gatccgagggcatgcgctgca

SEQ ID NO: 258 - DNA, 13 bp, Artificial Sequence
  Oligonucleotide
gcgcatgccctcg

SEQ ID NO: 259 - DNA, 19 bp, Artificial Sequence
  Oligonucleotide
gatccggctgcagggagct

SEQ ID NO: 260 - DNA, 11 bp, Artificial Sequence
  Oligonucleotide
ccctgcagccg

SEQ ID NO: 261 - DNA, 20 bp, Artificial Sequence
  Oligonucleotide
gatccgggctgcagggagct

SEQ ID NO: 262 - DNA, 12 bp, Artificial Sequence
  Oligonucleotide
ccctgcagcccg

SEQ ID NO: 263 - DNA, 21 bp, Artificial Sequence
  Oligonucleotide
gatccgaggctgcagggagct

SEQ ID NO: 264 - DNA, 13 bp, Artificial Sequence
  Oligonucleotide
ccctgcagcctcg

SEQ ID NO: 265 - DNA, 15 bp, Artificial Sequence
  Oligonucleotide
gatccgctgcagagg

SEQ ID NO: 266 - DNA, 11 bp, Artificial Sequence
  Oligonucleotide
cctctgcagcg

SEQ ID NO: 267 - DNA, 16 bp, Artificial Sequence
  Oligonucleotide
gatccggctgcagagg

SEQ ID NO: 268 - DNA, 12 bp, Artificial Sequence
  Oligonucleotide
cctctgcagccg

SEQ ID NO: 269 - DNA, 17 bp, Artificial Sequence
  Oligonucleotide
gatccgagctgcagagg

SEQ ID NO: 270 - DNA, 13 bp, Artificial Sequence
  Oligonucleotide
cctctgcagctcg

SEQ ID NO: 271 - DNA, 15 bp, Artificial Sequence
  Oligonucleotide
gatccggctgcaggc

SEQ ID NO: 272 - DNA, 15 bp, Artificial Sequence
  Oligonucleotide
tcgagcctgcagccg

SEQ ID NO: 273 - DNA, 16 bp, Artificial Sequence
  Oligonucleotide
gatccgggctgcaggc

SEQ ID NO: 274 - DNA, 16 bp, Artificial Sequence
  Oligonucleotide
tcgagcctgcagcccg

SEQ ID NO: 275 - DNA, 17 bp, Artificial Sequence
  Oligonucleotide
gatccgaggctgcaggc

SEQ ID NO: 276 - DNA, 17 bp, Artificial Sequence
  Oligonucleotide
tcgagcctgcagcctcg